(19) United States
(12) Patent Application Publication
(10) Pub. No.: US 2003/0036176 A1
(43) Pub. Date: Feb. 20, 2003

(54) Directed genetic engineering of xanthomonas campestris

(76) Inventors: Stanley G. Bower, La Jolla, CA (US); Thomas M. Ramseier, Poway, CA (US)

Correspondence Address: Gail Wuellner, Monsanto Company, 800 North Lindbergh Blvd., St. Louis, MO, 63167, US

(21) Appl. No.: 09/927,827

(22) Filed: Aug. 10, 2001

Related U.S. Application Data
(60) Provisional application No. 60/279,493, filed on Mar. 28, 2001.

(51) Int. Cl. C12P 19/06; C07H 21/04; C12N 1/21; C08B 37/00

(52) U.S. Cl. 435/104; 435/252.3; 536/23.2; 536/114

(57) ABSTRACT

DNA and protein sequences from Xanthomonas campestris pv. campestris, which may be targeted for activity reduction or enhancement using directed genetic engineering. Transformed microorganisms having reduced activity of at least one protein, e.g. galactomannanase, can be provided by disrupting a gene encoding the protein or introducing antisense nucleic acid sequence, providing xanthan gum essentially free of galactomannanase, amylase, cellulase or protease activity.

### CROSS-REFERENCE TO RELATED APPLICATION

This application claims priority under 35 U.S.C. §119(e) of U.S. Provisional Application No. 60/279,493 filed Mar. 28, 2001, the disclosure of which application is incorporated herein by reference in its entirety.

### BACKGROUND

Xanthan gum is a biosynthetic polysaccharide produced from glucose or other sugars by various bacterial species of the Xanthomonas genus e.g. Xanthomonas campestris pv campestris (herein after X. campestris). This gum is also referred to as “Xanthomonas hydrophilic colloid,” or as “Xanthomonas heteropolysaccharide” or as a“Xanthomonas gum”. Before use, xanthan gum is purified e.g. separated from bacterial contaminants. Xanthan gum preparation is described in U.S. Pat. Nos. 3,557,016; 3,481,889, 3,438,915 and 3,305,016, incorporated herein by reference in their entirety. Xanthan gum is widely used for a variety of commercial applications including food, oil field and other industrial uses.

Xanthan gum imparts a unique combination of texture, organoleptic properties and stability to foods. In foods, xanthan gum provides stability and improves or modifies textural qualities, pouring characteristics, and cling. In beverages, a slight increase in viscosity imparts the sensation of enhanced body without reducing flavor impact. Partially replacing high concentrations of starch in many food systems with xanthan gum contributes to a more pseudoplastic rheology; the benefits are improved flavor release and more pleasing texture. The synergistic reactivity of xanthan gum with galactomannans further expands its application potential.

Industrial applications of xanthan gum utilize its ability to provide formulations with properties such as long-term suspension and emulsion stability in alkaline, acid and salt solutions, temperature resistance and pseudoplasticity. Optimal fluids for oilfield uses have low viscosity at high shear rates (such as at the drilling bit) and high viscosity at low shear rates (as in the annular region). Xanthan gum solutions provide these properties. Differentiated xanthan gums emphasis properties such as solution clarity, low shear dispersion and enhanced acid stability.

For many applications, purified xanthan gum is mixed with other polysaccharides, e.g. mannans such as galactomannan. Xanthan gum is used in combination with mannans to make aqueous gels used in food, explosives and air treatment products. The combination is also used in the manufacture of controlled release oral solid dosage of pharmaceuticals. These other polysaccharides can be degraded by specific enzymes which reduce desired properties of a blended gum. Thus, when xanthan gum is mixed with galactomannans, the presence of the enzyme galactomannanase in the xanthan gum is undesirable.

Efforts to provide differentiated xanthan gum based on genetic alterations (U.S. Pat. No. 5,514,791, incorporated herein by reference in its entirety) and efforts to broaden the range of appropriate substrates for Xanthomonas fermentations by classical selection (U.S. Pat. No. 4,444,792, incorporated herein by reference in its entirety) and genetic engineering (Fu and Tseng (1990) Appl. Environ. Microbiol. 56(4):919-923, incorporated herein by reference in its entirety) have been described.

Previously, undesirable properties in xanthan gum were removed using chemical mutagenesis. However, because this type of mutagenesis is non-specific, chemically-mutagenized Xanthomonas strains that lacked a fully active enzyme of interest such as galactomannanase often exhibited decreased xanthan gum yield as well.

Knowledge of the gene set present in X. campestris pv.campestris as disclosed in U.S. application Ser. No. 09/703,708 (incorporated herein by reference in its entirety) allows directed genetic engineering to decrease or increase specific protein production. For example, undesired activities of specific enzymes such as galactomannanase can be reduced or eliminated in xanthan gum. Other target enzymes can include amylase, cellulase, extracellular protease, intracellular protease, and glucose dehydrogenase. Amylase is used by X. campestris to sacchrify corn syrups that are not already completely hydrolyzed; residual amylase could modify xanthan gum formulations containing corn syrup. Cellulase is used by X. campestris to digest cellulose in plant derived complex nitrogen sources; residual cellulase could modify xanthan gum formulations containing carboxymethyl cellulose. Extracellular and intracellular proteases are used by X. campestris to digest protein in complex nitrogen sources; residual protease could modify xanthan gum formulations containing proteinaeous material. The activity of glucose dehydrogenase diverts carbon from gum formation and acidifies the medium requiring neutralization which results in the accumulation of salt in the product; removal of its activity could improve xanthan gum quality.

Certain enzymes can be targeted for overexpression, e.g. enzymes of commercial significance such as galactomannanase, e.g. for paper bleaching applications, amylase, cellulase and extracellular protease.

A particularly preferred object of this invention is to use directed genetic engineering to “knock out” specific enzymes in Xanthomonas.

Another preferred object of this invention is to provide a recombinant strain of Xanthomonas campestris that is deficient in activity of at least one of the enzymes responsible for undesirable properties in xanthan gum.

### SUMMARY OF THE INVENTION

This invention provides transformed cells and organisms having reduced activity of at least one protein which is functionally equivalent to at least one of a galactomannanase, amylase, cellulase, extracellular protease, intracellular protease, and glucose dehydrogenase. Such peptides are functionally equivalent to wild-type proteins having at least 65 percent or higher similarity, more preferably at least 75 percent or higher similarity, even more preferably at least 90 percent or higher similarity to the amino acid sequence selected from the group consisting of SEQ ID NOs: 3 and 44 through 69. The reduced activity can be effected by the presence of anti-sense nucleic acid sequence or by modification of the nucleic acid sequence of the gene encoding said protein, e.g. providing said cell or organism with a recombinant nucleic acid sequence having at least one change as compared to a wild-type gene encoding said protein. For instance, the nucleic acid sequence encoding the protein can be reduced or increased by at least one nucleotide base, can be shuffled and/or can have at least one point mutation as compared to a wild-type gene encoding said protein. In certain aspects of this invention the nucleic acid sequence encoding the protein is reduced by two or more nucleotide bases as compared to a wild-type, even more preferably by a substantial amount, e.g. a major amount. In more preferred aspects of the invention substantially all of the nucleic acid sequence encoding the protein is deleted from the genome of the cell or organism.

This invention also provides a cell or organism having enhanced activity of at least one protein which is functionally equivalent to at least one of a galactomannanase, amylase, cellulase, extracellular protease and intracellular protease. Such peptides are functionally equivalent to wild-type proteins having at least 50 percent or higher similarity, more preferably at least 75 percent or higher similarity, even more preferably at least 90 percent or higher similarity to the amino acid sequence selected from the group consisting of SEQ ID NO:3 and SEQ ID NOs: 44 through 68. Enhanced activity can be achieved by providing the cell or organism with (a) multiple recombinant copies of the nucleic acid sequence of the gene encoding the protein, (b) recombinant regulatory sequence operably linked to a gene encoding the protein, or (c) shuffled nucleic acid sequence as compared to a wild-type gene encoding the protein. In preferred aspects of this invention the nucleic acid sequence of the wild-type gene will have at least 80 percent identity with a nucleic acid sequence selected from the group consisting of SEQ ID NOs: 2 and 18 through 42.

Another aspect of this invention provides DNA constructs useful for preparing the recombinant cells or organisms with reduced or enhanced protein activity.

In a preferred aspect of this invention the organism having reduced or enhanced protein activity is a recombinant bacteria, e.g. a recombinant Xanthomonas campestris bacteria. A preferred aspect of this invention provides a method for producing xanthan gum which is substantially free of certain protein activity, e.g. galactomannanase activity amylase activity, cellulase activity, extracellular protease activity, intracellular protease activity and/or glucose dehydrogenase activity . Such xanthan gum can be harvested from a cultured recombinant Xanthomonas campestris bacteria modified according to this invention.

### BRIEF DESCRIPTION OF THE DRAWINGS

FIG. 1 shows a multiple sequence alignment of parts of three mannan endo-1,4-beta-mannosidases.

FIG. 2 is a schematic representation of the suicide vector pTR213-b.

FIG. 3 illustrates the construction of the allele exchange suicide plasmid pBL170 for deletion of manA.

FIG. 4 illustrates allele exchange by “cross-in cross-out” via homologous recombination.

FIG. 5 shows position of PCR primers for evaluation of manA gene in knock-out candidates and the expected lengths of PCR products.

### DETAILED DESCRIPTION OF PREFERRED EMBODIMENTS

A. Definitions

As used herein “reduced protein activity” in a recombinant cell or organism is determined by reference to a wild-type cell or organism and can be determined by direct or indirect measurement. Direct measurement of protein activity might include an analytical assay for the protein, per se, or enzymatic product of protein activity. Indirect assay might include measurement of an property affected by the protein. For instance in the case of galactomannanase activity can be conveniently measured by locust bean gum (LBG) viscosity loss in a xanthan gum composition, e.g. because galactomannanase enzymatically reduces locust bean gum. Desired levels of reduced protein activity will vary depending on the application and protein being reduced. In the case of xanthan gum production from a culture of recombinant Xanthomonas campestris with reduced galactomannanase activity the recombinant organism will have at least a 99% reduction in galactomannanase activity, more preferably a 99.9% reduction and even more preferably at least 99.99% reduction in galactomannanase activity as measured by a LBG viscosity loss assay as discussed in the examples below.

A protein activity may be reduced by a variety of mechanisms. Antisense RNA will reduce the level of protein expressed and the activity will be reduced as compared to wild-type expression levels. Alternately, a mutation in the gene coding for a protein may not decrease the protein expression, but instead interfere with the protein's function to cause reduced protein activity.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. “Percent identity” is the identity fraction times 100.

Useful methods for determining sequence identity are disclosed in Guide to Huge Computers, Martin J. Bishop, ed., Academic Press, San Diego, 1994, and Carillo, H., and Lipton, D., SIAM J Applied Math (1988) 48:1073. More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990); version 2.0 or higher of BLAST programs allows the introduction of gaps (deletions and insertions) into alignments; BLASTX can be used to determine sequence identity between a polynucleotide sequence query and a protein sequence database; and, BLASTN can be used to determine sequence identity between sequences.

For purposes of this invention “percent identity” shall be determined using BLASTX version 2.0.14 (default parameters), BLASTN version 2.0.14, or BLASTP 2.0.14.

As used herein “peptide” means a compound with two or more amino acids linked in series by the carboxyl group of one amino acid to the amino group of the adjacent. “Polypeptide” means a peptide having at least 10 amino acids and includes proteins and protein fragments. Polypeptides which are not 100% sequence identical can be functionally equivalent because of conservative amino acid substitutions or because a segment of the protein performs the desired function. Polypeptides of the present invention also include protein homologs. Particularly preferred protein homologs are selected from the group consisting of bacteria such as E. coli, Bacillus thuringiensis, and other microorganisms such as yeast and Aspergillus nidulans.

As used herein the term “functionally equivalent” as applied to peptides means that functionally equivalent peptides perform the same function in nature, albeit at different levels of activity.

“Conservative amino acid substitutions” refer to substitutions of one or more amino acids in a peptide sequence with another amino acid(s) having similar side chains, resulting in a silent change. Conserved substitutes for an amino acid within a native wild-type amino acid sequence can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Naturally conservative amino acids substitution groups are: valine-leucine, valine-isoleucine, phenylalanine-tyrosine, lysine-arginine, alanine-valine, aspartic acid-glutamic acid, and asparagine-glutamine.

Polynucleotides can have sequence variability yet code for a functionally equivalent peptides due to codon degeneracy, conservative amino acid substitutions, reading frame positioning and the like. The term “codon degeneracy” refers to divergence in the genetic code permitting variation of the nucleotide sequence without effecting the amino acid sequence of an encoded polypeptide. The amino acid changes may be achieved by changing the codons of the polynucleotide sequence, e.g. according to the RNA codons given in the following Table.

TABLE 1

| Amino Acids / abbreviations | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The skilled artisan is well aware of the “codon-bias” exhibited by a specific host cell in usage of nucleotide codons to specify a given amino acid. Therefore, when identifying a gene, e.g. for deletion to reduce protein activity or synthesizing a gene for ectopic activity in a host cell to enhance protein activity, it is useful to include in the possible nucleic acid sequences to be used one having a nucleic acid sequence with a frequency of codon usage which approaches the frequency of preferred codon usage of the host cell.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2:482-489, 1981) to find the best segment of similarity between two sequences.

As used herein the term “antisense” refers to a polynucleotide molecule with a nucleic acid sequence which is complementary to a specific nucleic acid sequence. The term “antisense strand” is used in reference to a nucleic acid strand that is complementary to the “sense” strand. Antisense molecules may be produced by any method including synthesis or transcription. A complementary “antisense” molecule introduced into a cell can hybridize with a transcribed polynucleotide, i.e. mRNA, forming duplexes which block either further transcription or translation. The designation “negative” can refer to the antisense strand, and the designation “positive” can refer to the sense strand.

As used herein, a nucleic acid molecule and/or polypeptide molecule, be it a naturally occurring molecule or otherwise, may be “substantially purified”, if the molecule is separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

As used herein, the term “biologically active,” refers to a peptide having structural, regulatory, or biochemical functions of a naturally occurring molecule.

As used herein, the term “recombinant” refers to (a) molecules that are constructed outside of living cells by joining natural or synthetic DNA segments to DNA molecules that can replicate in a living cell, (b) molecules that result from the replication or expression of those molecules described in (a) above or (c) organisms that contain recombinant DNA or are modified using recombinant DNA, e.g. knock-out vectors.

As used herein, “disrupted/disruption” means that the gene does not encode or express wild-type peptide or encodes non-functional peptide or peptide having substantially reduced activity. Examples of a disrupted gene include genes with DNA deleted or inserted, and point mutations.

As used herein “flanking region” means the DNA on at least one side of a gene. Flanking regions are used for example in knock-out constructs used to delete all or a part of a wild-type gene sequence from the chromosome of a cell or organism.

Variations in peptide activity can be achieved by mutagenesis; screening methods for obtaining a specified protein or enzymatic activity of interest are disclosed in U.S. Pat. No. 5,939,250, the entirety of which is incorporated herein by reference. An alternative approach to the generation of variants uses random recombination techniques such as “DNA shuffling” as disclosed in U.S. Pat. Nos. 5,605,793; 5,811,238; 5,830,721; 5,837,458 and International Applications WO 98/31837 and WO 99/65927, the entirety of all of which is incorporated herein by reference. An alternative method of molecular evolution involves a staggered extension process (StEP) for in vitro mutagenesis and recombination of polynucleotide sequences, as disclosed in U.S. Pat. No. 5,965,408 and International Application WO 98/42832, the entirety of all of which is incorporated herein by reference. Other in vitro recombination methods are disclosed in U.S. patent application Ser. No. 09/746,432, the entirety of which is incorporated herein by reference.

As used herein, the term “manA gene” means a DNA sequence that encodes a functional galactomannanase enzyme. Other genes useful in this invention are described in the table below.

TABLE 2

| | | | | |
|---|---|---|---|---|
| SEQ | | coding | PEP | |
| NUM | SEQ ID | sequence | NUM | Description |

Amylases

| | | | | |
|---|---|---|---|---|
| 18 | XAN10 | 1001-2461 | 44 | alpha-amylase (EC 3.2.1.1) - Xanthomonas |
| | C691:94 | | | campestris gb\|AAA27591.1\| (M85252) alpha- |
| | 53_1291 | | | amylase [Xanthomonas campestris] |
| | 3RC | | | |
| 19 | XAN10 | 1001-2713 | 45 | alpha-amylase (EC 3.2.1.1) - |
| | C760:60 | | | Thermoactinomyces vulgaris |
| | 715_644 | | | emb\|CAA49465.1\| (X69807) alpha-amylase |
| | 27RC | | | [Thermoactinomyces vulgaris] |
| Cellulases | | | | |
| 20 | XAN10 | 1001-1957 | 46 | ENDOGLUCANASE PRECURSOR (ENDO- |
| | C680:40 | | | 1,4-BETA-GLUCANASE) (CELLULASE) |
| | 97_7053 | | | pir\|\|A42649 cellulase (EC 3.2.1.4) precursor - |
| | | | | Pseudomonas solanacearum gb\|AAA61980.1\| |
| | | | | (M84922) beta-1,4-endoglucase [Ralstonia |
| | | | | solanacearum] |
| 21 | XAN10 | 1001-2668 | 47 | N/A |
| | C683:11 | | | |
| | 459_151 | | | |
| | 26 | | | |
| 22 | XAN10 | 1001-2749 | 48 | MAJOR EXTRACELLULAR |
| | C684:77 | | | ENDOGLUCANASE PRECURSOR (ENDO- |
| | 8_4526 | | | 1,4-BETA-GLUCANASE) (CELLULASE) |
| | RC | | | pir\|\|JH0158 cellulase (EC 3.2.1.4) precursor - |
| | | | | Xanthomonas campestris pv. campestris |
| | | | | gb\|AAA27612.1\| (M32700) major extracellular |
| | | | | endoglucanase (engXCA) precursor |
| | | | | [Xanthomonas campestris] |
| 23 | XAN10 | 1001-2668 | 49 | EXOGLUCANASE A PRECURSOR |
| | C684:29 | | | (EXOCELLOBIOHYDROLASE A) (1,4- |
| | 44_6611 | | | BETA-CELLOBIOHYDROLASE A) |
| | RC | | | (CBP95) pir\|\|S49541 cellulase - Cellulomonas |
| | | | | fimi gb\|AAC36898.1\| (L25809) cellulase |
| | | | | [Cellulomonas fimi] |
| 24 | XAN10 | 1001-2608 | 50 | MAJOR EXTRACELLULAR |
| | C689:14 | | | ENDOGLUCANASE PRECURSOR (ENDO- |
| | 407_180 | | | 1,4-BETA-GLUCANASE) (CELLULASE) |
| | 14 | | | pir\|\|JH0158 cellulase (EC 3.2.1.4) precursor - |
| | | | | Xanthomonas campestris pv. campestris |
| | | | | gb\|AAA27612.1\| (M32700) major extracellular |
| | | | | endoglucanase (engXCA) precursor |
| | | | | [Xanthomonas campestris] |
| 25 | XAN10 | 1001-2002 | 51 | ENDOGLUCANASE PRECURSOR (ENDO- |
| | C618:3 | | | 1,4-BETA-GLUCANASE) (CELLULASE) |
| | 52_3353 | | | pir\|\|A42649 cellulase (EC 3.2.1.4) precursor - |
| | | | | Pseudomonas solanacearum gb\|AAA61980.1\| |
| | | | | (M84922) beta-1,4-endoglucanase [Ralstonia |
| | | | | solanacearum] |
| 26 | XAN10 | 1001-2008 | 52 | ENDOGLUCANASE PRECURSOR (ENDO- |
| | C618:1 | | | 1,4-BETA-GLUCANASE) (CELLULASE) |
| | 804_481 | | | pir\|\|A42649 cellulase (EC 3.2.1.4) precursor - |
| | 1 | | | Pseudomonas solanacearum gb\|AAA61980.1\| |
| | | | | (M84922) beta-1,4-endoglucanase [Ralstonia |
| | | | | solanacearum] |
| Extracellular Proteases | | | | |
| 27 | XAN10 | 1001-2032 | 53 | EXTRACELLULAR METALLOPROTEASE |
| | C665:1 | | | PRECURSOR pir\|\|A41048 extracellular |
| | _2670R | | | metalloproteinase (EC 3.4.24.-) precursor - |
| | C | | | Erwinia carotovora subsp. Carotovora |
| | | | | gb\|AAA24858.1\| (M36651) extracellular |
| | | | | protease (prt) [Pectobacterium carotovorum] |
| 28 | XAN10 | 1001-1870 | 54 | PROBABLE PROTEASE HTPX pir\|\|H64088 |
| | C679:1 | | | heat shock protein htpX - Haemophilus |
| | 7909_20 | | | influenzae (strain Rd KW20) gb\|AAC22378.1\| |
| | 778RC | | | (U32755) heat shock protein (htpX) |
| | | | | [Haemophilus influenzae Rd] |
| 29 | XAN10 | 1001-2743 | 55 | EXTRACELLULAR PROTEASE |
| | C690:4 | | | PRECURSOR pir\|\|S11890 serine proteinase |
| | 862_860 | | | (EC 3.4.21.-) precursor, extracellular - |
| | 4 | | | Xanthomonas campestris pv. Campestris |
| | | | | emb\|CAA35962.1\| (X51635) protease |
| | | | | [Xanthomonas campestris] |
| 30 | XAN10 | 1001-2452 | 56 | EXTRACELLULAR PROTEASE |
| | C690:8 | | | PRECURSOR pir\|\|S11890 serine proteinase |
| | 825_122 | | | (EC 3.4.21.-) precursor, extracellular - |
| | 76 | | | Xanthomonas campestris pv. Campestris |
| | | | | emb\|CAA35962.1\| (X51635) protease |
| | | | | [Xanthomonas campestris] |
| 31 | XAN10 | 1001-2650 | 57 | (S51030) serine protease, AspA [Aeromonas |
| | C700:1 | | | salmonicida, ssp. Salmonicida, Peptide, 621 |
| | 472_512 | | | aa] prf\|\|1907163A Ser protease [Aeromonas |
| | 1RC | | | salmonicida salmonicida] |
| 32 | XAN10 | 1001-2044 | 58 | N/A |
| | C731:46 | | | |
| | 4_3507 | | | |
| | RC | | | |
| 33 | XAN10 | 1001-2788 | 59 | Subtilisin BPN (E.C.3.4.21.14) |
| | C747:4 | | | |
| | 8081_51 | | | |
| | 868RC | | | |
| 34 | XAN10 | 1001-2884 | 60 | EXTRACELLULAR PROTEASE |
| | C757:1 | | | PRECURSOR pir\|\|S11890 serine proteinase |
| | 8772_22 | | | (EC 3.4.21.-) precursor, extracellular - |
| | 655 | | | Xanthomonas campestris pv. Campestris |
| | | | | emb\|CAA35962.1\| (X51635) protease |
| | | | | [Xanthomonas campestris] |
| Intracellular Proteases | | | | |
| 35 | XAN10 | 1001-3880 | 61 | (L43135) protease [Methylobacterium |
| | C685:56 | | | extorquens] |
| | 96_1057 | | | |
| | 5RC | | | |
| 36 | XAN10 | 1001-2287 | 62 | ATP-DEPENDENT CLP PROTEASE ATP- |
| | C702:17 | | | BINDING SUBUNIT CLPX pir\|\|A48709 |
| | 36_5022 | | | ATP-dependent clp proteinase (EC 3.4.21.-) |
| | RC | | | regulatory chain X - Escherichia coli |
| | | | | gb\|AAA16116.1\| (L18867) ATP-dependent |
| | | | | protease ATPase subunit [Escherichia coli] |
| | | | | gb\|AAB40194.1\| (U82664) ATP-dependent |
| | | | | Clp proteinase [Escherichia coli] |
| | | | | gb\|AAC73541.1\| (AE000150) ATP-dependent |
| | | | | specificity component of clpP serine protease, |
| | | | | chaperone [Escherichia coli K12] |
| 37 | XAN10 | 1001-1618 | 63 | ATP-DEPENDENT CLP PROTEASE |
| | C702:31 | | | PROTEOLYTIC SUBUNIT |
| | 47_5764 | | | (ENDOPEPTIDASE CLP) (CASEINOLYTIC |
| | RC | | | PROTEASE) (PROTEASE TI) (HEAT |
| | | | | SHOCK PROTEIN F21.5) pir\|\|B36575 ATP- |
| | | | | dependent clp proteinase (EC 3.4.21.-) chain P |
| | | | | - Escherichia coli gb\|AAA23588.1\| (J05534) |
| | | | | ATP-dependent protease (clpP) [Escherichia |
| | | | | coli] gb\|AAB40193.1\| (U82664) ATP- |
| | | | | dependent Clp proteinase [Escherichia coli] |
| | | | | gb\|AAC73540.1\| (AE000150) ATP-dependent |
| | | | | proteolytic subunit of clpA-clpP serine |
| | | | | protease, heat shock protein F21.5 [Escherichia |
| | | | | coli K12] |
| 38 | XAN10 | 1001-1576 | 64 | hypothetical 20.3 kD protein in sohA-mtr |
| | C722:15 | | | intergenic region - Escherichia coli (strain K- |
| | 344_179 | | | 12) gb\|AAA57956.1\| (U18997) ORF_o186 |
| | 19 | | | [Escherichia coli] gb\|AAC76187.1\| |
| | | | | (AE000396) orf, hypothetical protein |
| | | | | [Escherichia coli K12] |
| 39 | XAN10 | 1001-2977 | 65 | (M31045) ClpA protein [Escherichia coli] |
| | C756:44 | | | |
| | 20_8396 | | | |
| 40 | XAN10 | 1001-1978 | 66 | (M31045) ClpA protein [Escherichia coli] |
| | C756:57 | | | |
| | 26_8703 | | | |
| 41 | XAN10 | 1001-2353 | 67 | CARBOXY-TERMINAL PROCESSING |
| | C773:39 | | | PROTEASE PRECURSOR (C-TERMINAL |
| | 836_431 | | | PROCESSING PROTEASE) gb\|AAB61766.1\| |
| | 88RC | | | (L37094) protease [Bartonella bacilliformis] |
| 42 | XAN10 | 1001-3184 | 68 | TAIL-SPECIFIC PROTEASE PRECURSOR |
| | C779:66 | | | (PROTEASE RE) (PRC PROTEIN) |
| | 396_705 | | | pir\|\|A41798 carboxy-terminal proteinase (EC |
| | 79 | | | 3.4.21.-) precursor - Escherichia coli |
| | | | | gb\|AAA24699.1\| (M75634) tail-specific |
| | | | | protease [Escherichia coli] dbj\|BAA15638.1\| |
| | | | | (D90826) Tail-specific protease precursor (EC |
| | | | | 3.4.21.-) (Protease RE) (PRC protein). |
| | | | | [Escherichia coli] gb\|AAC74900.1\| |
| | | | | (AE000277) carboxy-terminal protease for |
| | | | | penicillin-binding protein 3 [Escherichia coli |
| | | | | K12] |
| Glucose Dehydrogenase | | | | |
| 43 | XAN10 | 1001-3412 | 69 | GLUCOSE DEHYDROGENASE |
| | C711:13 | | | [PYRROLOQUINOLINE-QUINONE] |
| | 605_180 | | | pir\|\|JV0107 glucose dehydrogenase |
| | 16 | | | (pyrroloquinoline-quinone) (EC 1.1.99.17) - |
| | | | | Escherichia coli dbj\|BAA05580.1\| (D26562) |
| | | | | ‘glucose dehydrogenase (pyrroloquinoline- |
| | | | | quinone)’ [Escherichia coli] gb\|AAC73235.1\| |
| | | | | (AE000122) glucose dehydrogenase |
| | | | | [Escherichia coli K12] |

Table headings:
SEQ NUM is the SEQ ID NO of the polynucleotide in the sequence listing
SEQ ID is an arbitrary name
coding sequence gives the location of the region of the polynucleotide which is translated into a protein as determined by a gene-predicting program.
PEP NUM provides the sequence listing number for the translations of the coding sequences described immediately above.
Description When a coding region is revealed by a homology based gene prediction method, the sequence to which it shows homology receives a description based on its comparison to another sequence in a public database after BLAST or a similar algorithm is used. The description that query sequence receives is listed under this column heading.

B. Reduced Protein Activity

This invention provides transformed cells and organisms having reduced activity of at least one protein which is functionally equivalent to at least one of a galactomannanase, amylase, cellulase, extracellular protease, intracellular protease, and glucose dehydrogenase. Such peptides are functionally equivalent to wild-type proteins having at least 50 percent or higher similarity, more preferably at least 75 percent or higher similarity, even more preferably at least 90 percent or higher similarity to the amino acid sequence selected from the group consisting of SEQ ID NOs: 3 and 44 through 69. The reduced activity can be effected by the presence of anti-sense nucleic acid sequence or by modification of the nucleic acid sequence of the gene encoding said protein, e.g. providing said cell or organism with a recombinant nucleic acid sequence having at least one change as compared to a wild-type gene encoding said protein. For instance, the nucleic acid sequence encoding the protein can be reduced or increased by at least one nucleotide base, can be shuffled and/or can have at least one point mutation as compared to the wild-type gene encoding said protein. In certain aspects of this invention the nucleic acid sequence encoding the protein is reduced by two or more nucleotide bases as compared to the wild-type, even more preferably by a substantial amount, e.g. a major amount. In more preferred aspects of the invention substantially all of the nucleic acid sequence encoding the protein is deleted from the genome of the cell or organism.

A recombinant organism can be prepared by any of a variety of ways known to those skilled in the art, e.g. by homologous recombination using DNA constructs for providing a modified sequence or knocking out the wild-type sequence. Sequence for knocking out a gene can comprise a modified wild-type gene, e.g. part of the gene sequence with an interim gap, or preferably flanking sequence for the gene where the interim gap comprises the gene per se. Preferably the construct comprises flanking sequence from both sides of the gene encoding the protein to be reduced, e.g. about 30 base pairs of flanking sequence from each side of the gene. Alternatively, the construct can comprise exogenous nucleic acid sequence flanked by sequence from said gene. In the case of knocking out a gene encoding a galactomannanase from Xanthomonas campestris, it is useful for the flanking sequences to comprise SEQ ID NOs: 6 and 7.

C. Enhanced Protein Activity

A further aspect of this invention provides a cell or organism having enhanced activity of at least one protein which is functionally equivalent to at least one of a galactomannanase, amylase, cellulase, extracellular protease and intracellular protease. Such peptides are functionally equivalent to wild-type proteins having at least 50 percent or higher similarity, more preferably at least 75 percent or higher similarity, even more preferably at least 90 percent or higher similarity to the amino acid sequence selected from the group consisting of SEQ ID NOs: 3 and 44 through 69. Enhanced activity can be achieved by providing the cell or organism with (a) multiple recombinant copies of the nucleic acid sequence of the gene encoding the protein, (b) recombinant regulatory sequence operably linked to a gene encoding the protein, or (c) shuffled nucleic acid sequence as compared to the wild-type gene encoding the protein. In preferred aspects of this invention the nucleic acid sequence of a wild-type gene will have at least 80 percent identity with a nucleic acid sequence selected from the group consisting of SEQ ID NOs: 2 and 18 through 43.

DNA constructs for producing the transformed cell or organism with enhanced activity of protein can comprise at least one modified sequence of a wild-type gene or a regulatory region operably linked to a wild-type gene. Targets for overexpression have desirable characteristics, for example, overexpression of mannanase is useful in bleaching paper, see U.S. Pat. Nos. 5,854,047 and 5,661,021, the entireties of all of which is incorporated herein by reference. In a preferred aspect of this invention the DNA construct will comprise a nucleic acid molecule having at least 85 percent sequence identity with SEQ ID NOs: 2 and 18 through 43.

Another aspect of this invention provides methods for producing a transformed cell or organism having enhanced activity of at least one protein comprising transforming the cell or organism with a construct of this invention.

D. DNA Constructs of the Invention

The present invention also encompasses the use of nucleic acids of the present invention in recombinant constructs. Using methods known to those of ordinary skill in the art, a protein encoding sequence and/or regulatory sequence of the invention can be deleted by inserting flanking regions into constructs which can be introduced into a Xanthomonas strain for the purpose of causing homologous recombination.

Furthermore, constructs may include those in which a Xanthomonas galactomannanase protein encoding sequence or portion thereof of the present invention is positioned with respect to a promoter sequence such that production of antisense mRNA complementary to native mRNA molecules is provided. In this manner, expression and activity of the native gene may be decreased. The present invention also encompasses the use of nucleic acids of the present invention in constructs which provide for mutation of genes within Xanthomonas by homologous recombination. Such constructs, for example, may contain two regions of a protein encoding sequence harboring a heterologous portion of DNA (such as an antibiotic resistance marker) between the two encoding segments. Such constructs may also contain, for example, other deletions, insertions, or base changes, or combinations thereof, relative to the Xanthomonas-derived DNA sequence. DNA shuffling may be used to modify the gene sequence to cause increased or decreased protein activity of the expressed protein. Introduction of these constructs into a target organism, e.g. Xanthomonas, can be used to generate mutations in the DNA of that organism. Such directed mutations are useful, for example, for controlling activity of mutated genes e.g. reduction of galactomannanase activity by disruption of the manA gene in X. campestris.

A further aspect of the present invention relates to recombinant vectors comprising nucleic acid molecules of the present invention. In a preferred embodiment a recombinant vector includes at least one nucleic acid molecule of the present invention which can preferably be (a) homologous to regions flanking a protein encoding region of this invention or fragment or homolog thereof, (b) homologous to regions flanking a regulatory element, promoter or partial promoter, or (c) antisense to manA mRNA or regulatory region or (d) homologous to a protein encoding region. In a further preferred embodiment of the present invention, a recombinant vector includes a regulatory element, promoter or partial promoter and a protein encoding region of the present invention, such nucleic acid molecules of the present invention having a sequence identified by SEQ ID NOs: 2 and 18 through 43 or complements thereof or fragments of either encoding proteins having an amino acid sequence of SEQ ID NOs: 3 and 50 through 69. In another preferred embodiment of the present invention, the recombinant vector includes a regulatory element, promoter or partial promoter and a nucleic acid molecule encoding a X. campestris protein homolog or fragments thereof. Preferably, such recombinant vectors of the present invention are introduced into a Xanthomonas species cell, more preferably a X. campestris cell, particularly a X. campestris strain NRRL B-1459 cell. It is also understood that such recombinant vectors may be introduced into any other cell or organism, including a plant cell, plant, fungal cell, fungus, mammalian cell, mammal, fish cell, fish, bird cell, bird or other (non-Xanthomonas) bacterial cell, so long as appropriate components, such as functional promoters, replication elements, and selectable markers are selected for the particular host to be transformed.

The recombinant vector of this invention may be any vector which can be conveniently subjected to recombinant DNA procedures. The choice of a vector will typically depend on the compatibility of the vector with the host cell into which the vector is to be introduced. The vector may be a linear plasmid or a closed circular plasmid. Examples of a method for homologous recombination using a linear vector is electroporation of linear DNA and a defective lambda prophage as described in Yu, Daiguan et al. (2000) Proc. Natl. Acad. Sci. USA, 97:5978-5983 or linear DNA and phage lambda Red recombinase, see Wanner, Barry et al. (2000) Proc. Natl. Acad. Sci. U.S.A., 97: 6640-6645. The vector system may be a single vector or plasmid or two or more vectors or plasmids which together contain the total DNA to be introduced into the genome of the host. Methods of introduction of recombinant vectors into Agrobacterium species have been described and include triparental mating (Ditta et al. (1985) Plasmid 13:149-153; Ditta et al. (1980) Proc. Natl. Acad. Sci. USA 77:7347-7351) and electroporation (White et al. (1995) Meth. in Mol. Biol. 47:135-141).

The vectors of the present invention preferably contain one or more selectable markers which permit easy selection of transformed cells. A selectable marker is a gene whose product provides, for example, biocide or viral resistance, resistance to heavy metals, prototrophy to auxotrophs, and the like. Various selectable markers may be used depending upon the host species to be transformed, and different conditions for selection may be used for different hosts.

Those vectors of the present invention used for homologous recombination are preferably suicide vectors, see U.S. Pat. No. 4,634,678, incorporated herein by reference in its entirety. As used herein “suicide vector” means a vector without an origin of replication or a vector with an origin of replication that does not work in the target organism (it may be an E. coli origin of replication for amplification of the plasmid prior to use in the target organism which is not E. coli).

A nucleic acid sequence of the present invention may be operably linked to a suitable promoter sequence. A nucleic acid molecule of the present invention which encodes a protein or fragment thereof may also be operably linked to a suitable leader sequence. A leader sequence may be a nontranslated region of an mRNA which is important for translation by a host cell. A leader sequence is operably linked to the 5′ terminus of the nucleic acid sequence encoding the protein or fragment thereof. The leader sequence may be native to the nucleic acid sequence encoding the protein or fragment thereof or may be obtained from foreign sources. A polyadenylation sequence may also be operably linked to the 3′ terminus of the nucleic acid sequence of the present invention, particularly for use in eukaryotic host cells.

To avoid the necessity of disrupting the cell to obtain the protein or fragment thereof, and to minimize the amount of possible degradation of the expressed protein or fragment thereof within the cell, it may be preferred that expression of the protein or fragment thereof gives rise to a product secreted outside the cell, especially in the case of expression in bacterial host cells of bacterium or bacteria. To this end, the protein or fragment thereof of the present invention may be linked to a signal peptide at the amino terminus of the protein or fragment thereof. A signal peptide is an amino acid sequence which permits the secretion of the protein or fragment thereof from the host into the culture medium.

A polypeptide encoding a nucleic acid molecule of the present invention may also be linked to a propeptide coding region. A propeptide is an amino acid sequence found at the amino terminus of apoprotein or proenzyme. Cleavage of the propeptide from the proprotein yields a mature biochemically active protein. The resulting polypeptide is known as a propolypeptide or proenzyme (or a zymogen in some cases). Propolypeptides are generally inactive and can be converted to mature active polypeptides by catalytic or autocatalytic cleavage of the propeptide from the propolypeptide or proenzyme. The propeptide coding region may be native to the protein or fragment thereof or may be obtained from foreign sources.

A polypeptide of the present invention may also be linked to a transit peptide coding region. A transit peptide is an amino acid sequence found at the amino terminus of an active protein which provides for transport of the protein into a plastid organelle, such as a plant chloroplast. The transit peptide coding region may be native to the type of cell to be transformed, or may be obtained from foreign sources.

An expressed polypeptide of the present invention may be detected using methods known in the art that are specific for the particular polypeptide. These detection methods may include the use of specific antibodies, formation of an enzyme product, or disappearance of an enzyme substrate. For example, if the polypeptide has enzymatic activity, an enzyme assay may be used. Alternatively, if polyclonal or monoclonal antibodies specific to the polypeptide are available, immunoassays may be employed using the antibodies to the polypeptide. The techniques of enzyme assay and immunoassay are well known to those skilled in the art.

The resulting polypeptide may be recovered by methods known in the arts. For example, the polypeptide may be recovered from the nutrient medium by conventional procedures including, but not limited to, centrifugation, filtration, extraction, spray-drying, evaporation, or precipitation. The recovered polypeptide may then be further purified by a variety of chromatographic procedures, e.g., ion exchange chromatography, gel filtration chromatography, affinity chromatography, or the like.

E. Recombinant Microorganisms

The present invention encompasses the use of recombinant microorganisms with modified (reduced or enhanced) protein activity. In a preferred aspect of the invention the micoorganism has reduced protein acitivity of at least one protein selected from the group having the function of at least one of galactomannanase, amylase, cellulase, extracellular protease, intracellular protease and glucose dehydrogenase. In a preferred aspect of this invention the organism having modified activity is a recombinant bacteria, e.g. a recombinant Xanthomonas campestris bacteria. The reduction in translated protein activity by the transformed cell or organism is measured by reference to a wild-type cell or organism. In the case of Xanthomonas campestris the reference organism is conveniently Xanthomonas campestris strain NRRL-B 1459. A preferred embodiment of the present invention is a recombinant Xanthomonas campestris strain comprising a specific targeted deletion of the manA coding region from the genome as exemplified in the examples herein as strain GMAN.

F. Method of Producing Xanthan Gum Using a Recombinant Organism

The fermentation of a culture of Xanthomonas campestris to produce xanthan gum is disclosed in U.S. Pat. No. 4,282,321, the entirety of which is incorporated herein by reference. Increased xanthan concentrations are obtained in Xanthomonas fermentations by addition of a source of assimilable carbon, e.g. glucose, corn syrup, etc., to an aqueous nutrient medium during the course of a fermentation cycle. See also U.S. Pat. Nos. 4,154,654; 4,394,447; 5,610,037; 5,756,317 and 6,033,896, the entireties of which are incorporated herein by reference.

### REFERENCES

Each reference mentioned in this specification is incorporated by reference in its entirety. In addition, these references, as well as each of those cited can be relied upon to make and use aspects of the invention.

### EXAMPLE 1

This example serves to illustrate the identification of a galactomannanase gene in X. campestris. More particularly the following procedure demonstrates a TBLASTN homology search using the protein sequence of galactomannanase from two other organisms to query the X. campestris genome.

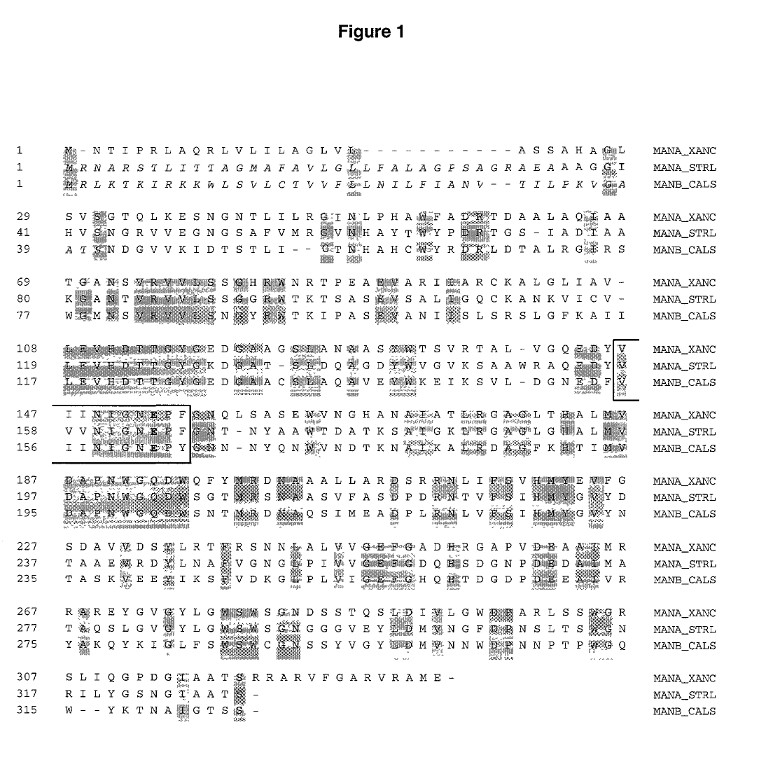

The sequence of two galactomannanases, i.e. from Caldocellum saccharolyticum (GeneBank:L01257.1) and Streptomyces lividans (GeneBank:M92297), are used to query the genomic sequence of Xanthomonas campestris. Each sequence is found to be most similar to the same open reading frame within contig XAN10C621 (SEQ ID NO: 1) of the X. campestris genome which is identified the “manA” gene and has the nucleic acid sequence of SEQ ID NO: 2. The X. campestris manA gene encodes a putative protein of 333 amino acids (called “MANA_XANCA” in FIG. 1) having the amino acid sequence of SEQ ID NO: 3. The X. campestris codon frequency was obtained from the Internet site, Kazusa (www.kazusa.or.jp/codon/) and utilized with the GCG program Codon Preference. X. campestris manA appears to be a single gene that is not part of an operon because the genes predicted to flank manA within contig XAN10C621 (SEQ ID NO 1) are transcribed from the opposite strand as manA precluding the possibility that they could be co-transcribed with manA. manA is preceded by a poor ribosome binding site (CTGGAG, bp 3616-3621 of SEQ ID NO 1) and followed by a stem-loop structure (bp 4652-4682) with a ΔG=−10.2 Kcal/mol.

As shown in Table 3, pair-wise comparisons of MANA_XANCA with the galactomannanases from S. lividans (MANA_STRLI having the amino acid sequence of Swiss Prot: P51529) and C. saccharolyticum (MANB_CALSA having the amino acid sequence of Swiss Prot: P22533) were generated with GCG program BestFit using the BLOSUM62 scoring matrix. The numbers reported are % identity and, in parenthesis, % similarity.

TABLE 3

| Proteins | MANA_STRLI | MANB_CALSA |
|---|---|---|
| MANA_XANCA | 55.1 (61.7) | 50.0 (58.6) |
| MANA_STRLI | — | 55.9 (62.8) |

From Table 3, it is apparent that the three proteins are ≧50% identical to each other.

A multiple sequence alignment of the three predicted protein sequences is shown in FIG. 1. The multiple sequence alignment comprises polypeptide sequences of MANA_XANCA (SEQ ID NO: 3), MANA_STRLI (SEQ ID NO: 16) and the beta-mannanase domain of MANB_CALSA (SEQ ID NO: 17). Amino acid residues that are conserved in all sequences are shaded. The indicated signal sequences for S. lividans MANA_STRLI and C. saccharolyticum MANB_CALSA are italized. The identified glucosyl hydrolase family 5 signature sequences (PROSITE accession number PS00659) are boxed. The conserved glutamate (E) present within this signature sequence is believed to be the active site residue (shown in bold).

### EXAMPLE 2

This example serves to illustrate a method for disrupting the function of the manA gene identified in Example 1. More particularly, the manA gene is disrupted by deletion using a suicide plasmid containing DNA regions flanking the manA gene.

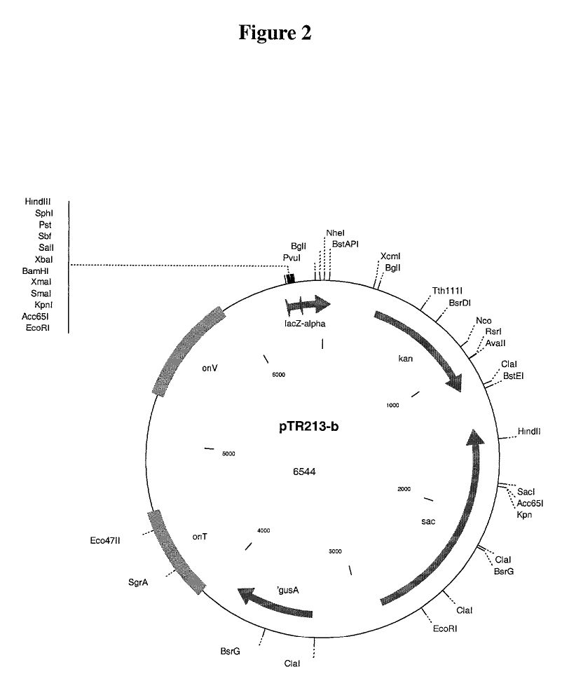

Allele exchange to result in the deletion of the manA gene of X. campestris pv campestris was accomplished in three steps: 1) construction of a suicide plasmid containing the regions flanking manA while omitting the manA coding region, 2) integration of the suicide plasmid at the homologous chromosomal locus and 3) excision of the manA gene and the vector sequence by a reciprocal homologous recombination. The suicide vector, pTR213-b, (FIG. 2) was derived from pK19mobGII (Katzen, F., A. Becker, M. V. Ielmini, C. G. Oddo, and L. Ielpi. Appl. Environ. Microbiol. 65(1) 278-282 (1999)) by removal of the center portion of gusA with introduction of the B. subtilis sacB gene (Gay, P., D. LeCoq, M. Steinmetz, E. Farrari, and J. A. Hoch. Cloning structural gene sacB, which codes for exoenzyme levansucrase of Bacillus subtilis: expression of the gene in Escherichia coli. J. Bacteriol. 153(3) 1424-1431). pTR213-b contains the kanamycin resistance gene from Tn5, the B. subtilis sacB gene imparting sensitivity to sucrose, a multiple cloning site, and the oriV of pMB1 which will replicate independently in E. coli but will not replicate independently in X. campestris.

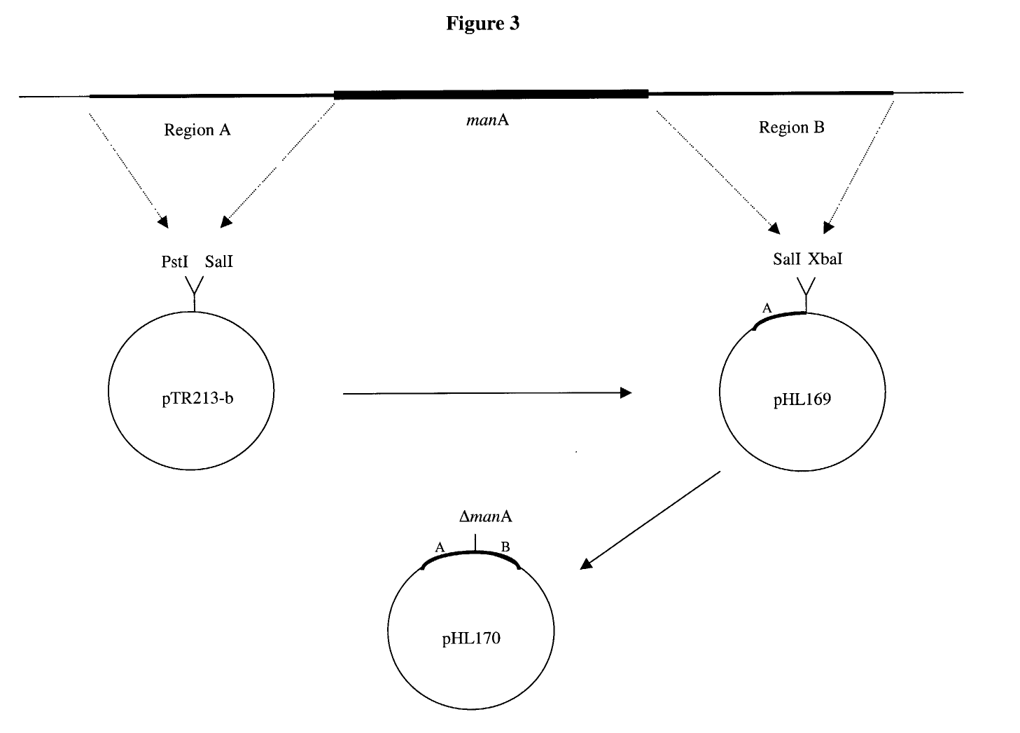

With reference to FIG. 3, the first step involves generation by PCR of the regions A and B which flank manA and cloning the regions A and B into the plasmid in an orientation replicating that of the chromosome, omitting the coding frame of manA. Region A (SEQ ID NO: 6) represents the 5′ flanking region which is upstream of the manA gene and Region B (SEQ ID NO: 7) represents the 3′ flanking region which is downstream of the manA gene. After identifying the X. campestris manA gene illustrated in Example 1, a DNA plasmid was constructed to disrupt the single chromosomal manA gene in a strain of X. campestris. The deletion is generated on the plasmid by cloning the two flanking DNA regions while omitting the coding region for manA (ΔmanA).

The DNA regions A and B flanking the manA gene but not the manA gene itself were amplified by PCR and cloned into a vector, pTR213-b. Region A was amplified by PCR utilizing primers pMANA-1F (SEQ ID NO: 8) and pMANA-1R (SEQ ID NO: 9) (see table 4 for a detailed description of primers) which introduced PstI and SalI sites into the amplified region A.

TABLE 4

| Seq | | Restriction | |
|---|---|---|---|
| Num | Seq ID | Sites | Comments |
| 8 | pMANA-1F | PstI | amplify region A upstream of manA. |
| 9 | pMANA-1R | SalI | amplify region A upstream of manA. |
| 10 | pMANA-2F | SalI | amplify region B downstream of |
| | | | manA. |
| 11 | pMANA-2R | XbaI | amplify region B downstream of |
| | | | manA. |
| 12 | P57manA5 | | 5′ Primer to amplify manA allele. |
| 13 | p58manA3 | | 3′ Primer to amplify manA allele |
| 14 | PmanA2F | | 5′ primer to amplify manA allele and |
| | | | utilized flanking sequence. |
| 15 | PmanA2R | | 3′ primer to amplify manA allele and |
| | | | utilized flanking sequence. |

Table headings:
Seq Num refers to the SEQ ID NO in the sequence listing.
Seq ID is an arbitrary name given to the primer.
Restriction Sites lists any restriction endonuclease sites designed into the primers.
Comments describes the use of the primer.

The amplified region A was digested with PstI and SalI and ligated to pTR213-b digested with the same restriction enzymes. XL-Blue E. coli cells were electroporated with a fraction of the ligation mix and kanamycin resistant colonies were selected. pHL169 was isolated from one of the kanamycin resistant colonies and demonstrated to be comprised of the expected 6.5 kb and 0.73 kb PstI-SalI fragments. pHL170 was constructed by addition of downstream region B juxtaposed to the upstream region A in pHL169. Region B (SEQ ID NO: 7) was generated by PCR utilizing primers pMANA-2F (SEQ ID NO 10) and pMANA-2R (SEQ ID NO 11) which introduced SalI and XbaI sites into the amplified region B. These sites allowed cloning of the downstream fragment in the same orientation as the upstream fragment into SalI, XbaI digested pHL169. After electroporation, kanamycin resistant XL-Blue E. coli candidates were identified which contained plasmid with the expected 7.3 kb and 0.7 kb fragments upon SalI, XbaI digestion; one plasmid isolate was named pHL170 (FIG. 3).

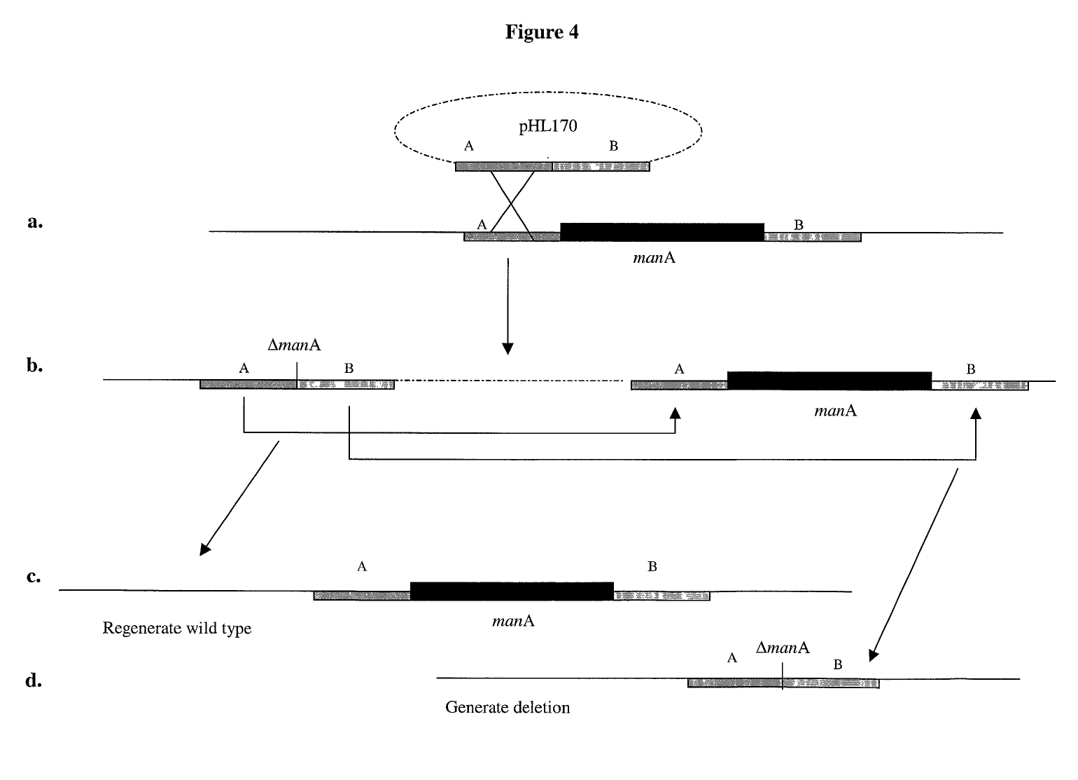

In the second step, transformed X. campestris candidates are generated by electroporation of the suicide knock-out plasmid containing both the upstream and downstream regions A and B flanking manA into the bacteria and selection by growing the target transformed X. campestris in the presence of kanamycin. FIG. 4a shows plasmid pHL170 with one possible alignment of a homologous region in the plasmid and the corresponding region in the bacterial chromosomal DNA. Those bacteria integrating a plasmid by homologous recombination will survive kanamycin selection while those bacteria without an integrated vector will die. The chromosomal structure in bacteria with an integrated plasmid will include a deleted manA locus, the integrated plasmid and a wild-type manA locus, as shown in FIG. 4b.

The plasmid pHL170 was introduced into a strain of Xanthomonas campestris by electroporation. X. campestris cells were grown to mid-log on TYE medium, collected by centrifugation and washed two times with de-ionized water. Cells were suspended in 1% of the initial volume of de-ionized water. pHL170 was purified from XL-Blue E. coli utilizing a Qiagen mini-spin kit. 3 μl of pHL170 were mixed with 50 μl of ice cold X. campestris electrocompetent cells in a 0.2 cm cuvet. The mixture was pulsed in a BioRad Pulser with nominal settings of 2.5 Kv, 1000 ohms, and 25 μF. The recorded pulse was 2.49 Kv, 22.5 msec. 2 ml of SOC at room temperature were immediately added and the mixture was incubated at 30° C., 250 rpm for 4 h. 10 μl and 100 μl aliquots were spread on TYE-Kan50 plates and incubated at 30° C. for two days. 86 kanamycin resistant candidates were generated by this process.

Many Gram negative bacteria have been demonstrated to develop sensitivity to sucrose if the heterologous gene sacB, derived from B. subtilis, is introduced. The kanamycin survivors were grown in the presence of sucrose to enrich for clones from which the integrated plasmid had been lost by a reciprocal homologous recombination event. Two results can be obtained from the reciprocal homologous recombination eliminating the plasmid. Either wild-type sequence, including a functional manA, remains (FIG. 4c), or the manufactured deletion remains in the chromosome (FIG. 4d). Screening by phenotype or direct examination of chromosomal structure by PCR can be used to differentiate these two results. Selection for elimination of the integrated suicide plasmid was accomplished by growth on sucrose; selecting against strains which had not undergone a second homologous recombination event to remove a plasmid. Kanamycin resistant candidates were grown under non-selective conditions; passage in TYE medium at 30° C. for two 24 hour cycles. Cultures were plated on 10% sucrose-TYE plates and colonies at two days were selected. 10% of the colonies proved kanamycin resistant (still contained integrated plasmid) and were discarded. Sucrose tolerant, kanamycin sensitive colonies were examined further.

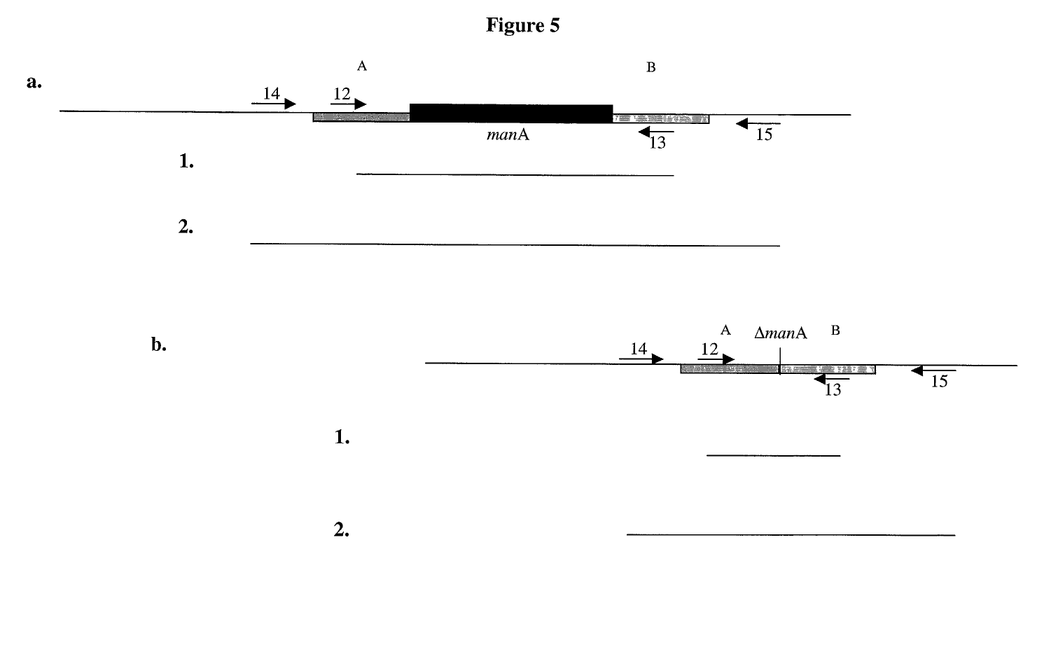

24 sucrose tolerant, kanamycin sensitive candidates were grown in liquid culture. Genomic DNA was prepared utilizing a MasterPure Kit (Epicentre, Madison, Wis. 53713) with conditions as recommended by the manufacturer. With reference to FIG. 5, PCR was utilized to examine colonies for deletion of the manA gene. Two sets of primers were utilized to identify the clones in which excision of the suicide plasmid gene resulted in the ΔmanA allele. The first set comprises primer P57manA3′ (SEQ ID NO: 12) and primer P58manA5′ (SEQ ID NO: 13) which lie within regions A and B. The second set comprises primer PmanA2F (SEQ ID NO: 14) and primer PmanA2R (SEQ ID NO: 15) which lie outside of regions A and B.

FIG. 5 shows where each primer set is designed to anneal to the wild-type manA allele (FIG. 5a) and to the recombinant ΔmanA allele (FIG. 5b). Using the first primer set (SEQ ID NOs: 12 and 13) the PCR products from wild-type allele should be about 1.4 Kb (FIG. 5.a.1) and the PCR products from the recombinant ΔmanA allele should be about 0.4 Kb (FIG. 5.b.1). Using the second primer set (SEQ ID NOs: 14 and 15) the PCR products from wild-type allele should be about 2.9 Kb (FIG. 5.a.2) and the PCR products from the recombinant ΔmanA allele should be about 1.9 Kb (FIG. 5.b.2).

PCR reactions contained 675 μl of Master Mix, 135 μl P58manA3, 135 μl P57manA5, 375 μl deionized water and 1 μl genomic DNA. The PCR program included a denaturation step at 95° C. for 15 minutes, 35 cycles of amplification with 1 minute at 95° C., 1 minute at 58° C., 1.5 minute at 72° C., a finishing step of 15 minutes at 72° C. and a quenching step at 4° C. Diluted pHL170 plasmid was used as a positive control (ΔmanA) and wild-type genomic DNA was utilized as a negative control (wild-type manA).

Fourteen isolates analyzed with the first set of primers yielded a single band of 0.4 kb indicating a ΔmanA allele while seven isolates provided a single 1.4 kb band indicating a wild-type manA allele. The remaining three isolates had multiple bands indicating mixed colonies or unexpected arrangements resulting from the constructions. The positive control, pHL170, gave the expected 0.4 kb band and the negative control, wild-type genomic DNA gave the expected 1.4 kb band.

To confirm the constructs had the engineered deletion integrated at the native locus of manA, using the second set of primers, PCR reactions were run as described above resulting in 1.9 kb PCR products for all strains which had been indicated as ΔmanA. Controls suggested to be wild-type at the locus were confirmed by the generation of 2.9 kb PCR products. The 14 confirmed ΔmanA isolates were named GMAN1 through GMAN14.

The GMAN strains are confirmed to deviate from wild-type by a chromosomal deletion of 1055 bp. This deletion is believed to encompass the entire manA gene, which is 1002 bp long, plus 2 bp of upstream flanking sequence and 51 bp of downstream flanking sequence.

### EXAMPLE 3

This example illustrates the reduced activity of galactomannanase of the GMAN strain of X. campestris compared to a wild-type strain by a plate assay designed to screen for the enzymatic activity.

Both strains (wild-type and GMAN) were grown on agar plates containing 9 grams per liter (g/L) of locust bean gum (LBG) as the main carbon source. LBG is degraded by X. campestris cells that express a functional galactomannanase. The resulting sugars, including glucose, are used by X. campestris to produce xanthan gum. A visual determination of gumminess of the colonies on the agar plate (as reported in Table 5) is an indication of function of the galactomannanase encoded by manA . Plates with 10 g/L glucose are included as a control to distinguish between isolates which cannot produce xanthan gum from simple saccharides and those which cannot produce xanthan gum from a galactomannan, e.g. LBG.

TABLE 5

| | Degree of gum formation on plates with: | |
|---|---|---|
| | | 9 g/L Locust Bean |
| | 10 g/L Glucose | Gum |
| Strains | (YM plates) | (LBG plates) |
| NRRL-B 1459 | ++++ | +++ |
| GMAN | ++++ | + |

+ very slightly gummy
++ slightly gummy
+++ gummy
++++ very gummy

NRRL-B1459, a wild-type X. campestris, isolated by the USDA Northern Regional Research Laboratory (available from the USDA Agricultural Research Service Culture Collection, Microbial Properties Research Unit, National Center for Agricultural Utilization Research, 1815 N. University Street, Peoria, Ill. 61604), demonstrates a “gummy” colony morphology in plates with either glucose or LBG as the primary carbon source. This indicates the strain is capable of degrading LBG into simple saccharides which can be utilized in xanthan gum production, i.e. the strain elaborates galactomannanase activity. GMAN produces a “gummy” phenotype on plates with glucose as the primary carbon source but appears only very slightly mucoid on plates with LBG as the carbon source. These results demonstrate that deletion of the manA gene substantially reduces the ability of the GMAN strains to utilize galactomannan while xanthan gum production from glucose is unimpaired.

### EXAMPLE 4

This example illustrates the use of the GMAN strain of Xanthomonas campestris in the production of xanthan gum. More particularly, the following illustrates reduced galactomannanase activity of the GMAN strain compared to a wild-type strain of X. campestris as measured by a viscosity loss assay.

The endolytic cleavage of galactomannan by galactomannanase results in a loss of viscosity. This viscosity loss is the basis of an assay for the galactomannanase. A solution of LBG is treated with broth from a X. campestris fermentation. The time dependent loss of viscosity is indicative of the amount of galactomannanase produced by the X. campestris.

The substrate solution was prepared by dissolving locus bean gum to a concentration of 1% in de-ionized water, addition of 0.2 volumes of 5% KH₂PO₄, pH 6.9 and pre-incubated at 40° C. Sample (1 ml of fermentation broth) or control (1 ml de-ionized water) was added to 100 ml of LBG substrate solution. The viscosity is measured with a Brookfield LVT viscometer (60 rpm, spindle #3) after 0, 3, 6 and 24 hours. Typical viscosity results are shown in Table 6 below.

TABLE 6

| | | Viscosity (cP) | | | |
|---|---|---|---|---|---|
| Strain | Dilution | 0 h | 3 h | 5 h | 24 h |
| NRRL-B | 10-1 | 560 | 17 | 10 | 10 |
| 1459 | 10-2 | 820 | 226 | 106 | 17 |
| | 10-3 | 820 | 570 | 480 | 230 |
| | 10-4 | 886 | 658 | 650 | 556 |
| GMAN#1 | none | 888 | 692 | 690 | 670 |
| GMAN#2 | none | 980 | 715 | 750 | 760 |
| GMAN#3 | none | 960 | 730 | 750 | 780 |
| DI water | — | 820 | 620 | 640 | 600 |

Results show that there is a reduction in viscosity over the first three hours when GMAN broth is added to LBG solution. There is no further loss over the next 21 hours. Comparison with the de-ionized water control demonstrates that the initial decrease is not related to galactomannanase in the sample. For comparison, dilutions, in de-ionized water, of fermentation broth from wild-type NRRL-B 1459 were assayed. Ten fold diluted broth from the wild-type strain eliminated essentially all viscosity within 3 hours. Even 10,000 fold diluted broth, in 24 hours, destroyed 16% of the viscosity after correcting for the viscosity loss observed with control over the first three hours. These data demonstrate a greater than 1000 fold reduction in endogalactomannanase activity in GMAN relative to a wild-type strain.

1. A transformed cell or organism having reduced activity of at least one protein wherein
a. a nucleic acid sequence of a gene encoding said protein is a recombinant sequence which is reduced by at least one nucleotide base as compared to a wild-type gene encoding said protein;
b. the activity of said protein is reduced by the presence of anti-sense nucleic acid sequence;
c. the nucleic acid sequence of the gene encoding said protein is a recombinant sequence having at least one point mutation as compared to the wild-type gene encoding said protein;
d. the nucleic acid sequence of the gene encoding said protein is a recombinant sequence which is shuffled as compared to the wild-type gene encoding said protein; or
e. the nucleic acid sequence of the wild-type gene encoding said protein is a recombinant sequence which is increased by at least one nucleotide base as compared to the wild-type gene encoding said protein;
and wherein the nucleic acid sequence of said wild-type gene encodes a peptide having at least 65 percent similarity with an amino acid sequence selected from the group consisting of SEQ ID NOs: 3 and 44 through 69.

2. A transformed cell or organism according to claim 1 wherein the transformed cell or organism is a recombinant Xanthomonas campestris bacteria.

3. Recombinant Xanthomonas campestris bacteria of claim 2 wherein the function of said protein is reduced to a level not more than about 0.1 percent of protein activity in said wild-type strain.

4. Recombinant Xanthomonas campestris bacteria of claim 3 wherein said wild-type strain is NRRL-B 1459.

5. Recombinant Xanthomonas campestris bacteria of claim 2 wherein the number of nucleotide bases in said gene is reduced or increased by two or more as compared to said wild-type gene.

6. Recombinant Xanthomonas campestris bacteria of claim 5 wherein said gene encodes a galactomannanase.

7. Recombinant Xanthomonas campestris bacteria of claim 6 wherein at least substantially all of said gene is deleted.

8. Recombinant Xanthomonas campestris bacteria of claim 7 wherein said bacteria is strain GMAN.

9. A DNA construct useful for generating a cell or organism according to claim 1 comprising at least one of (a) modified sequence of a wild-type gene and (b) flanking sequence of said wild-type gene.

10. A DNA construct of claim 9 comprising a vehicle for homologous recombination comprising flanking sequence from both sides of said gene.

11. A DNA construct according to claim 10 wherein said flanking sequence comprises at least about 30 base pairs of flanking sequence from each side of said gene.

12. A DNA construct of claim 10 1 wherein said flanking sequence comprises at least about 30 base pairs of SEQ ID NO: 4 and SEQ ID NO: 5.

13. A DNA construct of claim 11 wherein said flanking sequence comprises SEQ ID NO: 6 and SEQ ID NO: 7.

14. A DNA construct according to claim 9 wherein said modified sequence of a wild-type gene comprises an exogenous nucleic acid sequence flanked by sequence from said gene.

15. A transformed cell or organism having enhanced activity or function of at least one protein wherein
a. said cell or organism comprises multiple recombinant copies of a nucleic acid sequence of a gene encoding said protein;
b. said cell or organism comprises recombinant regulatory sequence operably linked to the gene encoding said protein, or
c. the nucleic acid sequence of the gene encoding said protein is shuffled as compared to a wild-type gene encoding said protein;
and wherein the nucleic acid sequence of said wild-type gene has at least 80 percent identity with a nucleic acid sequence selected from the group consisting of SEQ ID NOs: 2 and 18 through 42.

16. A DNA construct useful for producing the transformed cell or organism of claim 15 comprising at least one of (a) modified sequence of said wild-type gene and (b) a regulatory region operably linked to a said wild-type gene.

17. A DNA construct of claim 15 wherein the nucleic acid molecule has a sequence which has at least about 85% homology with SEQ ID NO:2.

18. A method of producing a transformed cell or organism having reduced activity of at least one protein comprising transforming said cell or organism with a construct according to claim 9.

19. A method of producing xanthan gum substantially free of galactomannanase activity comprising harvesting xanthan gum from cultured recombinant Xanthomonas campestris strain having more than one nucleotide base in a manA gene deleted.

20. A method of claim 19 wherein substantially all of a manA gene is deleted.

21. A xanthan gum which is essentially free of activity of at least two proteins selected from the group consisting of galactomannanase, amylase, cellulase, extracellular protease, intracellular protease, and glucose dehydrogenase due to recombinant manipulation of genes encoding said proteins, wherein said xanthan gum is harvested from a cultured recombinant Xanthomonas campestris.


SEQUENCE LISTING (69 sequences)

SEQ ID NO: 1 - DNA, 7356 bp, Xanthomonas campestris
  unsure (1)..(7356): unsure at all n locations
ctccctgcgcaacggcatctacaccatgccgcagttcctggagcagcgttacggcaagtggatccgcacgctgatggcggtgttctggctgctgctgtacgtgttcgtcaatctcacctcgatcctgtggctgggctcgatcgcggtcagccaggtcaccggcatggaccagaccctggcactgaccctgatcggcgtgttcgcgctggtgtatcagctgtacggcggtttgaaggccgtggccctgaccgacatcgtgcaggtcactttgctggtgttgggcggtctgctggtgaccggcctgacgttgtcgcgcatcggcgatggcgctggcgtgctggccggcttcaagcagctgtggagtgcgcacccggagcacttccacatgatcctcagcaaggacaacccgttctacaaggatctgcctggcctgagcgtgctgctgggcgggctgtgggtgatgaacatcagctactggggcttcaaccagtacatcatccagcgcgcactggccgccaagaacatcggtgaagcgcagaagggcatggtgttcgcggccttcctgaagctgctgatgccggtggtgatcgtggtgccgggcattgccgcagtggtgctggcaccggatctggccaagcctgatcaggcgtaccccaccatgatgcagctgttgccgaccggcatcctgggcctggtgtttgccgcgctggtggccgcgatcgtggcctcgctggcgtcgaagatcaattcggtggcgacgatctttaccctggatttctacgccaagttccgcccgcagaccgagcagaaacagctggtgcgcgtgggtcgcatcgttgccggcgtctcggtgctgatcggcatcctcaccgcacggccgttgctgggcaacttcgaccagggcttccagttcatccaggaattcaccggcttctttacccccggtgtggtggtgatcttcatgctggggctgttctggaagcgtgccaatgaagctggcgcattgactgcggcgatcggctcggtggtgctgtcgttcgcgctcaagatcctgtggccggaactgcccttcatggaccgcatcggcgtggtgttcgtcgccgcgctggtgctggcggtggtggtgtcgctcctgacaccggccacgcaggcacgcgatctgatccgcaccaaggacgtggcctacggcaccacgctgggcttcaagatcggcgcggtgggcgtggttgcgattctgatcgcgctgtacgcggtgttctggtaagcgccagcgcacatcacacgcaataagtaacgcggcgcggagagcattctctgcgccgcgttgcgtttgggcgccgtcactcagaggcgcgcggcttccagttgcggtgctggcggggcagcgtcggtggcgcgttggtaccacggtgtctccaaccaggcttccagcgctgcggcaggcatcggcttggcgatccagtagccctggccttcgtcgcagccccagctgcgcagctgtgcgtaggcctgcgccgactcgatcccttcggccaccacacgctggcccaggctgtggccgagctggatcatcgccggcaccagggtgcggtcggtgcggctgtccggcagcgagcggacgaaggattgatcgatcttcagcgagctggccggcagttgtttgaggtagctgaaattgctgtagccggtgccgaagtcgtcgatggcgatatgcacgcccagcgccgcaacggccgccagttgctcggacagatgctcggggtggcggatcatcgcgctttcggtgaattcgatctccagccggcgcgggtcgagcttgtggcggtccaggccgcggcgcagcaacccgacgaaaccgggttgatccagatcggcggccgacacgttcaaggccaggttgaagtccaggccctgctgttgccagcgtgcggcctgggcgatgccctggtcgatcacccaggcggtgatgcggttgatcagcgcggttttttcagccatcggaatgaagtccgacggcatcaccgggccgatcatcgggtgctgccagcgcagtagcgcttccaccgccacgcagcggtgatcgtgcagatccacgcgcggctggtagtgcaggcgcagctgattgctgctggccagcgcggcgggcagggcggccagcaggcggaaggtgttgcgctggaccacgtcgtgcttacgctcgtacaggctccaggtcagacaatgttcgcgggcgacatccactgaggtggtgagcgagcgcagcgtgtcggcggcgccgtagctggtttccagcaacaccgcgccgatcgaggcgaccgcggtatgcggaatgccctggtgctccagcggcgccacgaaccccttggaaatcttgctgcacagcgcgctcaggcgcagcgggtcggcggtggtgaccatgaaggcgaaggtggtcggatccagccggtacaaggcggtgccggccggcaatttgctgatcaaacgcttgtaggccagcgccacatagccgtctgcgtaatcccagcccagcgccttgaccatgtcgcggaaataatcgctgccgcacaggtccaccgcgatcgcggtggcggcggaatcggccgtgcgttgcagcaataccgcgtccaggtcttcgctgaagcgcgcgcggttgggcaggccggtggggccatcgcggtaggtactgctgcgcaggttttcgacccgcagcacggccaggtcgcgcaggccttccagttggatgcgttgctccgcatccagcccggcacgcgggttggtgccaatcacgcacagcgtgccgatggtgtggccatcgcgcagcttgagcggtgcgccagcgtaaaagcggatatgcggcgcacccagtaccagcgggttgttgcagaagcgcgggtccagcaaggcatcgggaatcaccaggacgtcatcggagcgcagcgcgtgcgcgcagaaggcatgttcgcgcggggtctcaggcgcttccaggccgacacgggctttgaaccactggcggtgctcgtccaccagcgagaccagggcgatctcggtccccagcatgcgtgcggccatggcggcgatggtgtcgaacaccgggtcgggcggcgagtccagcaggcaaagaccgtgcaggaccgccagccgtgccgcttcatcgagcgcagacgggaggggggaggcgacaggagcgggcgtggaatgcatgtgcccgtatcggcctcgagcgcggcgacttgatcacgcgcgactggcgccgttcagttggccgcgcgtgcgagtggtgcagcgcggcaatccgcacgctgcattcgacactccgcgcagcactggcggccgaatcactgcgctgcaccacggcagggcgcagacgggttcgggtgcgctgtgcctacagggccgagggtttgctcgccgtttgcaacgagcgcgtggtgcatggacacgcacgagataacgttaccagcacgctggatgcggatctgcgcgctctgtcctgatctgtacctgttgtcgattctgtgacccgcgctgcgatcgcctgcgtcgcgggttggcaaggtcttggcgcacgcctgatgcgcgctgtccgcggacgccggtctgcggcgtccaccttgtctggagccgagcatgaacaccattcctcgcctggcgcaacgcctggtcctgatccttgccggcttagtgctggcctcatccgcgcacgccggcttgtccgtgtcgggcacacagctgaaggaatccaatggcaataccttgatcctgcgcggcatcaacctgccgcatgcctggtttgccgatcgcaccgatgccgcgctggcgcagatcgcggccaccggtgccaacagcgtgcgggtggtgctcagttccggccaccgctggaaccgaacgcccgaggccgaggtggcgcgcatcatcgcgcgctgcaaggccctggggttgatcgcggtgctggaggtgcacgacaccaccggctatggcgaagacggcgccgccggcagcctggccaatgccgccagttactggaccagcgtgcgcactgcgctggtgggccaggaagactacgtgatcatcaacatcggcaacgagccgttcggcaatcagctcagtgccagcgaatgggtcaacggccacgccaatgcgatcgccacgctgcgtggcgccgggctcacgcatgcgctgatggtggatgcgcccaattggggccaggactggcagttctacatgcgcgacaacgcggcggcgttgctggcgcgcgacagccgccgcaacctgattttcagcgtgcacatgtacgaggtattcggcagcgacgcggtggtggacagctacctgcgcaccttccgcagcaacaatcttgcgctggtggtgggcgagttcggcgccgatcatcgcggcgcgcccgtggacgaggcggcgatcatgcgccgcgcgcgcgagtacggcgtgggctatctcggctggtcgtggtcgggcaacgacagcagcacgcagtcgctggatatcgtgctcggttgggatcctgcgcgtttgagcagttggggacgcagcctgattcagggcccggacggcattgccgcgacctcgcgccgtgcgcgggtgttcggtgcgcgcgtgcgggcgatggagtgatgcatcggtggttttgcggcggggcgcgatgtgcctgcaaggatcatcgcgtctgctgcgtttcgggcagtgtgttatgtgaggcgatatcgggtgagtgcagagttcgcaggccgcgcacttgatcggctggcagcctagatagggcgttcgacgactgcgctgtgtacgcaacgtcggttgtgcttcagcgcaaagacaaagactgacgcgctgtcatccatgagccgcggcgaacgatcacccatgcagaggcagccgcgcaacgtctttgcgaaagccggccaaaaccgcagcaggcacaccaggcgcttagcgcccgggttcgcgctgcggtcccgggcttgggcgcttggtgagcttgcgctgcagcgagcggcggtgcatgcccagcaggcgcgcggcggcggacacgttgccgccggtttcgtgcagcgcctgctggatgtgctcccactgcaggcggctgagcggggtcatcatttcctgcgcggtttcttcttcgtccggatcgggcaggtcgtcgtcctcttcgcccagcgcacgcatgatggtggggatgttggctggcttgggcaggtagtcgtcggcgccgagcttgatcgcctccaccgcggtggcgatgctcgcgtacccggtgaccagcaggatgcgcatgtccgggcggatttcgcgcagcggctggatcaggttcaatcccgagtcgtggcccagcttcagatcgatcaaggcgaagtccggcagcgcgctgcgtgcggtggacaaggcgctggccgcatcggtggcggtgagcgtttccacgccgcggcgggcgaggctgcgttgcaaggtacgcagatacagggtgtcgtcgtcgaccagcaggccggtgcggatgggagtgctcatgcaagggcctcgtgttcggagagcggcaggcggaaaccgacgcgggcgccgctgccttcggcgggaagcatccacagttcaccctgcagccgctcgacggtggcatgcgacagggccaggcccacgcccatgccgtcgggcttgccgctgttgaacaaggtgcccggcaacatcgcctgcgaggtgtcgaagcccagcccgtagtcgcgcacttcgccgaccagttggtcgtcttccacgcgcagggtgaggtcgatcagcggccggccggcgcgttcgccggcatcggcggcattgttgagcagcaccatcagcaggtggctcacgccgggctgcagcatcagccgcagcggcgcgtcttcattgcggcgcagctcgatggtcgggcgcaccaggcgccactgttccagcacatcgcgcaccgccacctcgcggctgagatggccgttgtcggccggtgctgccagcgccagcacgcgctcgtggcattgcaccagcagctcgcgcagggtttccaggtcttcgcgcaactcgacctgatcgcactggtcggcgatgtcgtcggccagcagggtcatcgtcgccagcggggtgttgagctcatgcgccaccgaggcggcatgcgtggccagcgccacgatgccttcgttgcgtgcaaagcgctcgcgcagtgtggagatctcgcgttcgcgctcgcgcagcgacaacgccagccgggtggcgaacaccagcaccaccacggtggaaagcaggaagttggccgccatgccccacatgtgcagggtcaacgggtcgaagccgccgtacggcagtggcaagccgaatgcggcgctgatgacgtagccggccacgcaggccgccgccaccgccatcgcccagctcagcggcagggcgagcgcggccagcgcgatcagcaccaggaacagcgaaccgaacgggttggcgatgccgccgctccagcccaccatccaggtcagcaccgtcacatcgaccaggatgtggccgaactcggtggccgggctgaccgnnccnnggtgcgccacccgcagctgcgcatcccgggcgcacaccggcggcgcggccaccccggcccaaagcggatcctgccgaagggagaggccaattacacccgtggcgagcaggatgggtggccgactgtccgagcgaggcctgttcaacgcatgcctgccagggtacggcaagaactgaagccggcttaaagtatttactgtttggtaataacttatgcgcttttgtccaggtgttgagttggccctttggggagcgagaacagggttctcaaatttacccgaattccccgcatagtgaggagcccccgaatgtttagccaattcccgtgttttttaacaccttttgttattgggcccctgggaaaccccgggctcctgactccccagtttcgtccgacctcttgtcaggtcccccgagaattgttttctgttaaattccccgggtttagggtccggatatttcaaaggtccgccccccccgaccgggaatctctaaggtggcccttgtttgaaccgtccccttgtttagtaaaccaggtccgtaataagaatttcgttaggctaaatttttttttggatcggtagccacagttgtgaaaggattttttttcgggttttatttggctaaacttcctgtggcttttgggggcccttttataccagcggtggccttggtcacctttataactatccccaaatgttttgagtggtgcttctcttattggtcaactcaggggtccctatttttaccctactccaaatctatatttcccccgctcggtggggtattggtcttattcctgggccg

SEQ ID NO: 2 - DNA, 1002 bp, Xanthomonas campestris
atgaacaccattcctcgcctggcgcaacgcctggtcctgatccttgccggcttagtgctggcctcatccgcgcacgccggcttgtccgtgtcgggcacacagctgaaggaatccaatggcaataccttgatcctgcgcggcatcaacctgccgcatgcctggtttgccgatcgcaccgatgccgcgctggcgcagatcgcggccaccggtgccaacagcgtgcgggtggtgctcagttccggccaccgctggaaccgaacgcccgaggccgaggtggcgcgcatcatcgcgcgctgcaaggccctggggttgatcgcggtgctggaggtgcacgacaccaccggctatggcgaagacggcgccgccggcagcctggccaatgccgccagttactggaccagcgtgcgcactgcgctggtgggccaggaagactacgtgatcatcaacatcggcaacgagccgttcggcaatcagctcagtgccagcgaatgggtcaacggccacgccaatgcgatcgccacgctgcgtggcgccgggctcacgcatgcgctgatggtggatgcgcccaattggggccaggactggcagttctacatgcgcgacaacgcggcggcgttgctggcgcgcgacagccgccgcaacctgattttcagcgtgcacatgtacgaggtattcggcagcgacgcggtggtggacagctacctgcgcaccttccgcagcaacaatcttgcgctggtggtgggcgagttcggcgccgatcatcgcggcgcgcccgtggacgaggcggcgatcatgcgccgcgcgcgcgagtacggcgtgggctatctcggctggtcgtggtcgggcaacgacagcagcacgcagtcgctggatatcgtgctcggttgggatcctgcgcgtttgagcagttggggacgcagcctgattcagggcccggacggcattgccgcgacctcgcgccgtgcgcgggtgttcggtgcgcgcgtgcgggcgatggagtga

SEQ ID NO: 3 - protein, 333 aa, Xanthomonas campestris
MNTIPRLAQRLVLILAGLVLASSAHAGLSVSGTQLKESNGNTLILRGINLPHAWFADRTDAALAQIAATGANSVRVVLSSGHRWNRTPEAEVARIIARCKALGLIAVLEVHDTTGYGEDGAAGSLANAASYWTSVRTALVGQEDYVIINIGNEPFGNQLSASEWVNGHANAIATLRGAGLTHALMVDAPNWGQDWQFYMRDNAAALLARDSRRNLIFSVHMYEVFGSDAVVDSYLRTFRSNNLALVVGEFGADHRGAPVDEAAIMRRAREYGVGYLGWSWSGNDSSTQSLDIVLGWDPARLSSWGRSLIQGPDGIAATSRRARVFGARVRAME

SEQ ID NO: 4 - DNA, 3627 bp, Xanthomonas campestris
ctccctgcgcaacggcatctacaccatgccgcagttcctggagcagcgttacggcaagtggatccgcacgctgatggcggtgttctggctgctgctgtacgtgttcgtcaatctcacctcgatcctgtggctgggctcgatcgcggtcagccaggtcaccggcatggaccagaccctggcactgaccctgatcggcgtgttcgcgctggtgtatcagctgtacggcggtttgaaggccgtggccctgaccgacatcgtgcaggtcactttgctggtgttgggcggtctgctggtgaccggcctgacgttgtcgcgcatcggcgatggcgctggcgtgctggccggcttcaagcagctgtggagtgcgcacccggagcacttccacatgatcctcagcaaggacaacccgttctacaaggatctgcctggcctgagcgtgctgctgggcgggctgtgggtgatgaacatcagctactggggcttcaaccagtacatcatccagcgcgcactggccgccaagaacatcggtgaagcgcagaagggcatggtgttcgcggccttcctgaagctgctgatgccggtggtgatcgtggtgccgggcattgccgcagtggtgctggcaccggatctggccaagcctgatcaggcgtaccccaccatgatgcagctgttgccgaccggcatcctgggcctggtgtttgccgcgctggtggccgcgatcgtggcctcgctggcgtcgaagatcaattcggtggcgacgatctttaccctggatttctacgccaagttccgcccgcagaccgagcagaaacagctggtgcgcgtgggtcgcatcgttgccggcgtctcggtgctgatcggcatcctcaccgcacggccgttgctgggcaacttcgaccagggcttccagttcatccaggaattcaccggcttctttacccccggtgtggtggtgatcttcatgctggggctgttctggaagcgtgccaatgaagctggcgcattgactgcggcgatcggctcggtggtgctgtcgttcgcgctcaagatcctgtggccggaactgcccttcatggaccgcatcggcgtggtgttcgtcgccgcgctggtgctggcggtggtggtgtcgctcctgacaccggccacgcaggcacgcgatctgatccgcaccaaggacgtggcctacggcaccacgctgggcttcaagatcggcgcggtgggcgtggttgcgattctgatcgcgctgtacgcggtgttctggtaagcgccagcgcacatcacacgcaataagtaacgcggcgcggagagcattctctgcgccgcgttgcgtttgggcgccgtcactcagaggcgcgcggcttccagttgcggtgctggcggggcagcgtcggtggcgcgttggtaccacggtgtctccaaccaggcttccagcgctgcggcaggcatcggcttggcgatccagtagccctggccttcgtcgcagccccagctgcgcagctgtgcgtaggcctgcgccgactcgatcccttcggccaccacacgctggcccaggctgtggccgagctggatcatcgccggcaccagggtgcggtcggtgcggctgtccggcagcgagcggacgaaggattgatcgatcttcagcgagctggccggcagttgtttgaggtagctgaaattgctgtagccggtgccgaagtcgtcgatggcgatatgcacgcccagcgccgcaacggccgccagttgctcggacagatgctcggggtggcggatcatcgcgctttcggtgaattcgatctccagccggcgcgggtcgagcttgtggcggtccaggccgcggcgcagcaacccgacgaaaccgggttgatccagatcggcggccgacacgttcaaggccaggttgaagtccaggccctgctgttgccagcgtgcggcctgggcgatgccctggtcgatcacccaggcggtgatgcggttgatcagcgcggttttttcagccatcggaatgaagtccgacggcatcaccgggccgatcatcgggtgctgccagcgcagtagcgcttccaccgccacgcagcggtgatcgtgcagatccacgcgcggctggtagtgcaggcgcagctgattgctgctggccagcgcggcgggcagggcggccagcaggcggaaggtgttgcgctggaccacgtcgtgcttacgctcgtacaggctccaggtcagacaatgttcgcgggcgacatccactgaggtggtgagcgagcgcagcgtgtcggcggcgccgtagctggtttccagcaacaccgcgccgatcgaggcgaccgcggtatgcggaatgccctggtgctccagcggcgccacgaaccccttggaaatcttgctgcacagcgcgctcaggcgcagcgggtcggcggtggtgaccatgaaggcgaaggtggtcggatccagccggtacaaggcggtgccggccggcaatttgctgatcaaacgcttgtaggccagcgccacatagccgtctgcgtaatcccagcccagcgccttgaccatgtcgcggaaataatcgctgccgcacaggtccaccgcgatcgcggtggcggcggaatcggccgtgcgttgcagcaataccgcgtccaggtcttcgctgaagcgcgcgcggttgggcaggccggtggggccatcgcggtaggtactgctgcgcaggttttcgacccgcagcacggccaggtcgcgcaggccttccagttggatgcgttgctccgcatccagcccggcacgcgggttggtgccaatcacgcacagcgtgccgatggtgtggccatcgcgcagcttgagcggtgcgccagcgtaaaagcggatatgcggcgcacccagtaccagcgggttgttgcagaagcgcgggtccagcaaggcatcgggaatcaccaggacgtcatcggagcgcagcgcgtgcgcgcagaaggcatgttcgcgcggggtctcaggcgcttccaggccgacacgggctttgaaccactggcggtgctcgtccaccagcgagaccagggcgatctcggtccccagcatgcgtgcggccatggcggcgatggtgtcgaacaccgggtcgggcggcgagtccagcaggcaaagaccgtgcaggaccgccagccgtgccgcttcatcgagcgcagacgggaggggggaggcgacaggagcgggcgtggaatgcatgtgcccgtatcggcctcgagcgcggcgacttgatcacgcgcgactggcgccgttcagttggccgcgcgtgcgagtggtgcagcgcggcaatccgcacgctgcattcgacactccgcgcagcactggcggccgaatcactgcgctgcaccacggcagggcgcagacgggttcgggtgcgctgtgcctacagggccgagggtttgctcgccgtttgcaacgagcgcgtggtgcatggacacgcacgagataacgttaccagcacgctggatgcggatctgcgcgctctgtcctgatctgtacctgttgtcgattctgtgacccgcgctgcgatcgcctgcgtcgcgggttggcaaggtcttggcgcacgcctgatgcgcgctgtccgcggacgccggtctgcggcgtccaccttgtctggagccgagc

SEQ ID NO: 5 - DNA, 2730 bp, Xanthomonas campestris
  unsure (1)..(2730): unsure at all n locations
tgatgcatcggtggttttgcggcggggcgcgatgtgcctgcaaggatcatcgcgtctgctgcgtttcgggcagtgtgttatgtgaggcgatatcgggtgagtgcagagttcgcaggccgcgcacttgatcggctggcagcctagatagggcgttcgacgactgcgctgtgtacgcaacgtcggttgtgcttcagcgcaaagacaaagactgacgcgctgtcatccatgagccgcggcgaacgatcacccatgcagaggcagccgcgcaacgtctttgcgaaagccggccaaaaccgcagcaggcacaccaggcgcttagcgcccgggttcgcgctgcggtcccgggcttgggcgcttggtgagcttgcgctgcagcgagcggcggtgcatgcccagcaggcgcgcggcggcggacacgttgccgccggtttcgtgcagcgcctgctggatgtgctcccactgcaggcggctgagcggggtcatcatttcctgcgcggtttcttcttcgtccggatcgggcaggtcgtcgtcctcttcgcccagcgcacgcatgatggtggggatgttggctggcttgggcaggtagtcgtcggcgccgagcttgatcgcctccaccgcggtggcgatgctcgcgtacccggtgaccagcaggatgcgcatgtccgggcggatttcgcgcagcggctggatcaggttcaatcccgagtcgtggcccagcttcagatcgatcaaggcgaagtccggcagcgcgctgcgtgcggtggacaaggcgctggccgcatcggtggcggtgagcgtttccacgccgcggcgggcgaggctgcgttgcaaggtacgcagatacagggtgtcgtcgtcgaccagcaggccggtgcggatgggagtgctcatgcaagggcctcgtgttcggagagcggcaggcggaaaccgacgcgggcgccgctgccttcggcgggaagcatccacagttcaccctgcagccgctcgacggtggcatgcgacagggccaggcccacgcccatgccgtcgggcttgccgctgttgaacaaggtgcccggcaacatcgcctgcgaggtgtcgaagcccagcccgtagtcgcgcacttcgccgaccagttggtcgtcttccacgcgcagggtgaggtcgatcagcggccggccggcgcgttcgccggcatcggcggcattgttgagcagcaccatcagcaggtggctcacgccgggctgcagcatcagccgcagcggcgcgtcttcattgcggcgcagctcgatggtcgggcgcaccaggcgccactgttccagcacatcgcgcaccgccacctcgcggctgagatggccgttgtcggccggtgctgccagcgccagcacgcgctcgtggcattgcaccagcagctcgcgcagggtttccaggtcttcgcgcaactcgacctgatcgcactggtcggcgatgtcgtcggccagcagggtcatcgtcgccagcggggtgttgagctcatgcgccaccgaggcggcatgcgtggccagcgccacgatgccttcgttgcgtgcaaagcgctcgcgcagtgtggagatctcgcgttcgcgctcgcgcagcgacaacgccagccgggtggcgaacaccagcaccaccacggtggaaagcaggaagttggccgccatgccccacatgtgcagggtcaacgggtcgaagccgccgtacggcagtggcaagccgaatgcggcgctgatgacgtagccggccacgcaggccgccgccaccgccatcgcccagctcagcggcagggcgagcgcggccagcgcgatcagcaccaggaacagcgaaccgaacgggttggcgatgccgccgctccagcccaccatccaggtcagcaccgtcacatcgaccaggatgtggccgaactcggtggccgggctgaccgnnccnnggtgcgccacccgcagctgcgcatcccgggcgcacaccggcggcgcggccaccccggcccaaagcggatcctgccgaagggagaggccaattacacccgtggcgagcaggatgggtggccgactgtccgagcgaggcctgttcaacgcatgcctgccagggtacggcaagaactgaagccggcttaaagtatttactgtttggtaataacttatgcgcttttgtccaggtgttgagttggccctttggggagcgagaacagggttctcaaatttacccgaattccccgcatagtgaggagcccccgaatgtttagccaattcccgtgttttttaacaccttttgttattgggcccctgggaaaccccgggctcctgactccccagtttcgtccgacctcttgtcaggtcccccgagaattgttttctgttaaattccccgggtttagggtccggatatttcaaaggtccgccccccccgaccgggaatctctaaggtggcccttgtttgaaccgtccccttgtttagtaaaccaggtccgtaataagaatttcgttaggctaaatttttttttggatcggtagccacagttgtgaaaggattttttttcgggttttatttggctaaacttcctgtggcttttgggggcccttttataccagcggtggccttggtcacctttataactatccccaaatgttttgagtggtgcttctcttattggtcaactcaggggtccctatttttaccctactccaaatctatatttcccccgctcggtggggtattggtcttattcctgggccg

SEQ ID NO: 6 - DNA, 731 bp, Xanthomonas campestris
gcaaggcatcgggaatcaccaggacgtcatcggagcgcagcgcgtgcgcgcagaaggcatgttcgcgcggggtctcaggcgcttccaggccgacacgggctttgaaccactggcggtgctcgtccaccagcgagaccagggcgatctcggtccccagcatgcgtgcggccatggcggcgatggtgtcgaacaccgggtcgggcggcgagtccagcaggcaaagaccgtgcaggaccgccagccgtgccgcttcatcgagcgcagacgggaggggggaggcgacaggagcgggcgtggaatgcatgtgcccgtatcggcctcgagcgcggcgacttgatcacgcgcgactggcgccgttcagttggccgcgcgtgcgagtggtgcagcgcggcaatccgcacgctgcattcgacactccgcgcagcactggcggccgaatcactgcgctgcaccacggcagggcgcagacgggttcgggtgcgctgtgcctacagggccgagggtttgctcgccgtttgcaacgagcgcgtggtgcatggacacgcacgagataacgttaccagcacgctggatgcggatctgcgcgctctgtcctgatctgtacctgttgtcgattctgtgacccgcgctgcgatcgcctgcgtcgcgggttggcaaggtcttggcgcacgcctgatgcgcgctgtccgcggacgccggtctgcggcgtccaccttgtctggagccgagca

SEQ ID NO: 7 - DNA, 746 bp, Xanthomonas campestris
atgcatcggtggttttgcggcggggcgcgatgtgcctgcaaggatcatcgcgtctgctgcgtttcgggcagtgtgttatgtgaggcgatatcgggtgagtgcagagttcgcaggccgcgcacttgatcggctggcagcctagatagggcgttcgacgactgcgctgtgtacgcaacgtcggttgtgcttcagcgcaaagacaaagactgacgcgctgtcatccatgagccgcggcgaacgatcacccatgcagaggcagccgcgcaacgtctttgcgaaagccggccaaaaccgcagcaggcacaccaggcgcttagcgcccgggttcgcgctgcggtcccgggcttgggcgcttggtgagcttgcgctgcagcgagcggcggtgcatgcccagcaggcgcgcggcggcggacacgttgccgccggtttcgtgcagcgcctgctggatgtgctcccactgcaggcggctgagcggggtcatcatttcctgcgcggtttcttcttcgtccggatcgggcaggtcgtcgtcctcttcgcccagcgcacgcatgatggtggggatgttggctggcttgggcaggtagtcgtcggcgccgagcttgatcgcctccaccgcggtggcgatgctcgcgtacccggtgaccagcaggatgcgcatgtccgggcggatttcgcgcagcggctggatcaggttcaatcccgagtcgtggcccagcttcagatcgatcaaggcgaagtccggcagc

SEQ ID NO: 8 - DNA, 30 bp, Xanthomonas campestris
aaaactgcagccagcaaggcatcgggaatc

SEQ ID NO: 9 - DNA, 30 bp, Xanthomonas campestris
acgagtcgactcggctccagacaaggtgga

SEQ ID NO: 10 - DNA, 30 bp, Xanthomonas campestris
acgagtcgactctgctgcgtttcgggcagt

SEQ ID NO: 11 - DNA, 30 bp, Xanthomonas campestris
ctagtctagaaacgctcaccgccaccgatg

SEQ ID NO: 12 - DNA, 19 bp, Xanthomonas campestris
ctacagggccgagggtttg

SEQ ID NO: 13 - DNA, 21 bp, Xanthomonas campestris
cagcgcgtcagtctttgtctt

SEQ ID NO: 14 - DNA, 23 bp, Xanthomonas campestris
cggtggggccatcgcggtaggta

SEQ ID NO: 15 - DNA, 24 bp, Xanthomonas campestris
cgtcgagcggctgcagggtgaact

SEQ ID NO: 16 - protein, 329 aa, Streptomyces lividans
MRNARSTLITTAGMAFAVLGLLFALAGPSAGRAEAAAGGIHVSNGRVVEGNGSAFVMRGVNHAYTWYPDRTGSIADIAAKGANTVRVVLSSGGRWTKTSASEVSALIGQCKANKVICVLEVHDTTGYGKDGATSLDQAGDYWVGVKSAAWRAQEDYVVVNIGNEPFGNTNYAAWTDATKSAIGKLRGAGLGHALMVDAPNWGQDWSGTMRSNAASVFASDPDRNTVFSIHMYGVYDTAAEVRDYLNAFVGNGLPIVVGEFGDQHSDGNPDEDAIMATAQSLGVGYLGWSWSGNGGGVEYLDMVNGFDPNSLTSWGNRILYGSNGIAATS

SEQ ID NO: 17 - protein, 325 aa, Caldocellum saccharolyticum
MRLKTKIRKKWLSVLCTVVFLLNILFIANVTILPKVGAATSNDGVVKIDTSTLIGTNHAHCWYRDRLDTALRGIRSWGMNSVRVVLSNGYRWTKIPASEVANIISLSRSLGFKAIILEVHDTTGYGEDGAACSLAQAVEYWKEIKSVLDGNEDFVIINIGNEPYGNNNYQNWVNDTKNAIKALRDAGFKHTIMVDAPNWGQDWSNTMRDNAQSIMEADPLRNLVFSIHMYGVYNTASKVEEYIKSFVDKGLPLVIGEFGHQHTDGDPDEEAIVRYAKQYKIGLFSWSWCGNSSYVGYLDMVNNWDPNNPTPWGQWYKTNAIGTSS

SEQ ID NO: 18 - DNA, 3461 bp, Xanthomonas campestris
  CDS (1001)..(2461)
atcagttcgaaaccctcgccgtagcgcgcgatcaccaacgcgccactggccagctggctgcgcaagagggggctgaccagcacgctgcgaatgacgctgccatcgttgaagcggtaatcgatctcgccttcgcgcttgacgcggctggtctcgatgatctggcggacctgcgccaccagttcctgctgccgcagctgcgcgttgcgctcctcggccaatgcgcggtcgcgctcggcgcgctcggcttgcaggcgggcggcatccaccttgtccgggtctgctgcggccgccggtgcacccttggcatggcgctgcttgttctgctcgcgcgccaccttgtccacctgggctttcttgaccaggcctgccttgagcagttgttcttgaagaggattgcgcatgcgagaaaaccaacggtaacgtgcacacagtttaacgcgaccggcgtggtggccgccgagcgcacaaggtgtcgctgccccaggaggcctgaaccaggctgttcaggcttagcgccgctatcaaaacgcctgcagccgccagccgggcgcggccggtgctaggaatcggcatgtacccccatacactccgggtgctccgcgccggccgcacccacctgacaacttactcgctacgtttcggtagccgctcttcataccgctgagccgttgtgcacgcgctgctgcactgggctgccgtcgttgcctgtagcgcgcaggttgcgccccgagcccggcctgctggacgtcacggcatccataccggcaaccaccgccgccattcatcagcaccgcgcgcaatgcgtcggcgcgagtgcgaccgtgcacgccctcggcatcgaaccggccgcacgtttcgcggctgttgtgcagtgaatacgtatgcacagggtcaatgctgcgccgcgatgcgcgcttagcatcaccgcgcgccgcacgccgtggggcgcgctttccggaacaggggccggagggcattgctgcatgcatcgctgtgcgggatgcgtgcgtccactgatcaggagacacacccgatgcacgccacctctcgcccctgtccacgcacgttctggcaacgcgctcaccagttgctgttgatcgcgctgaccttgttgctgaccaccgccagcgcacaggccgacgtcatcctgcatgcgttcaactggccctacgccacggtggaagcgcgcgccaagcagatcgccgatgccgggtatcgcaaggtactggtcgcaccggcgtatcgctccgaaggcagtgcctggtgggcgcgctaccagccgcaggatatccgcctgatcgacaatccactcggcgacaccaccgcgttcgcgcgcatggtgcaggcactggccaataacggcgtggaaacctatgccgacgtggtgttcaaccacatggccaacgaagcagcaacgcgctcggacctcaattaccccggcagcgcggtgctggcgcagtacgcggccaatcctgggcgctacgatgcactgcgcctgttcggcaccgtgcagtccaactttttgagcggcagtgatttcgggccggcgcaatgcatcagcaactacaacgatgccttccaggtgcgcaattaccgcatctgcggcggcggcagcgatccgggcctgccggatctgctcggcaacgattgggtggtgcagcagcagcgcgcctacctgcaggcgctcaagggcctgggcgtcaccggcttccgcgtggacgcggccaagcacatgaccttcgaccatctcaaccgcgtgttcgatgccggcatccgctccggtgtgtacgtgtttggtgaagtgatcaccggcggtggcagcggcaatggcgactacgaccagttcctggcgccgtatctgcaatccaccccgcatgccgcctacgatttcccgctgttcaacgcggtacgcacggccttcggtgtgggtgcgagcatgcaacagttggtggacccggcgtccgccgggcaggccttgccgggcaaccgtgcagtcacctttgccgtgacgcatgacatccccaataacgcgggcttccgctacgcgattctcgacccggtcgatgaaacgctggcctatgcgtacttgctgggccgcaacggcggcgtgccgatggtctacaccgacaacaacgaaagcggcgacaaccgctgggtcaatgcctatctgcgcgatgatctgcgccgcatgattggcttccacaacggcgtgcagggcagcgacatgcaggtgctgtcatccagtgcctgccacatcctgttccgccgcggcagcctcggcatcgtgggcatcaacaagtgcggcaacccggtcaacaccaccgtcgccatgaacggcagcgtgttgttctggaatgcggattacgtcgatgcactgggttccggcaccgtggtgcgcatctccagtggctcgtacaccttcaccctgccggcgcggcaggcgcgtatgtggcgacgttgattttgcagacgcgctgcagcacggtctgttttccgtaggtgacatccaggcaaccagggaacaccagcagatcccgctgagtagcaacaccgacgccttcatgggtgcagcccgcatgaaggcgtcgatgcgttaggcaaccgaaaaaatctacaacgacgcatctttttcattgctggcggcatttcaggaccacgcgatgacgcgcacggttaccagcaggcgccctcgcgcgcatgccaaccaaagatgaactgtccgcgcaccgcaacccatcaatgcaacccgttcgcacggcgcgcgtacctacgagcggtcgctgcaattgcttccttctgctgcgcggcgaccatcgtggcgagcgccatgcgagcactacacgcatgacatcgccctcccccatcgatggtgcacgccggagcatgcgatgcctctctactttgtccgccatggggaatcgctcgccaacgaacagaactacttcgctggttcccagaattcgccgctgacccggctcggccggcatcaggcgcagcaggccgcgctacgtgcagcaacgcgcactgcacttcgaccaggtgcatgtctccacgctggagcgcgcgcaggcaacggcagccatcatcctgcatgacgttgcgcccatgcccgaggtcgtttccagcgcggcactggtggaacgcaacttcggcatcttcgccggcaagaacaagaccttgatcaagaagtcggtcggccatgcggtgtttgaacgctacttccatgatgccgatggcgcgccaccggacggcgaacactggatggatatgtacgcccgctgcaagacctactacgagaccgtgctggcgccgctggatcagcaagccaagcacgtgctggtcgtcgcgcacaaatacatcgtggaagtcttagcgctgatcgcatccgggctgccgccagcggagtacatcgacttccgcttgcccaactcgcgcccgctgtcatgggacgaactcaagca

SEQ ID NO: 19 - DNA, 3713 bp, Xanthomonas campestris
  CDS (1001)..(2713)
tcattggcgtcggcgcgattgtggcgagctttctgccctggctgctgacgcgctggggcgtagacaacactgcacctgccggcgagctgcccgatagcgtgcgctacgcgttctacctaggtgcggcggtgttgttcctgtcgatcagctggacggtgctgcgcacgcgcgaatacagcccggccgagctggcggcgtttgaaccggcgccggcaccggctgccgcgcaactggccgccaccgatcaacccagcctgcaggccagcgtgctgtggtgtctgggcgggttgctgctggccgctgccatcgcctggcagcacggcgatcgcatgttgtacgtgctggcgggcctgtgcatcgcctatggcgtgctgctggcgttggcgcgcgtgctgccgcgtggcggcatgctggtcgcaatcatgcacgatctgcgccacatgccgcagacgatgcggcgcctggcctgggtgcagttcttttcgtggtttgcgctgtttgcgatgtggatctacaccaccgcggcagtgacgcaggtgcatttcggcgcacgcgataccgtctcggccgcctacaacgacggggcgaactgggtgggtgtgttgttcggcgcctacaacggctttgccgcgctcgccgccaccgtcatcccgctgatggtgcgtgcgatcgggctgcgctggagccacctgtgcaacctgtggctgggtgcggcggggttgttgtcgatgctgttcatccgcgacccgtactggctgctgctgtcgatgctgggcgtcggttttgcctgggcctcgatcctgtccttgccctatgcactgctgtccgacagcgtgcccgccgccaagatgggcgtgtatatgggcatcttcaatttctttatcgtgattccgcaattggttgccgccagcgccctgggcttcgtgctccgcgtgtggctgggcgggcaaccgatctacgcgctggcgatcggcggcttgagcctgatcgtggccggtgtgtgcgtggtgcgggtcccaagcgcccagggagggcaatgatgcgcagcgtcttgatggcagccatgctgctttattcgggagcagcctgcgctgcgcctgcacccggtgattactacggcaccctggaaccgttcgccgccgatgcggtgtatttcgtggtcaccgatcgcttcgtcaacggcgacacgggcaacgatcaccgcgaccagggcggcgcacatcgcagcttcgatgtgcccacaccgtgcgacggtggcgtgggcgacaacatcggttacctcggcggcgacttcaagggcatcgtcgatcatgccgactacattcgcggcctcggctttggcgcggtgtggatcaccccgatcgtcgacaacccggacgaagcgttcaccggtggcaagcccatcacctgcgagagcacgctcagcgatcacggcaagaccggttatcacggctattggggcgtcaatttttacaggctcgacgaacatctccccagcccggggctggatttcgccgggttcacgcgcgccatgcacgccaatgacctgaaggtggtgctggatatcgtcggcaaccacggctcgcccgcctacagcatgccggtggcgcagcccgggtttggcaagctctacgacgcacagggccgcctggttgccgatcatcagaacctcgcacccgcacagctcgacccggcgcacaacccgttgcacgcgttctacaacaccagcggcggcctggcggaattgtcggacctcaatgaagacaacccggctgtgctggactatctagccggcgcctacctgcagtggatggagcagggtgccgatgcgtttcgcatcgacaccatcggctggatgccagaccgcttctggcatgccttcgtagcgcggatccgcgagaaacgcccgggcgttttcatgtttggcgaagccttcgactacgacccggccaagatcgccggccacacctgggcgcgcaacgccggcgtcagcgtgctggacttcccgctcaaacagcagctatccgccgtgttcggccacaaacaggctggcttcgaacagctcgccacgccgctgtatctgcgcaaaggcccgtatggcaacccgtacgagttgatgagcttctacgacaaccacgacatggcgcgcctggatgccagcgataccggcttcatcgatgcgcacaattggttgttcaccgcgcgcggcattccggtgatctattacggctcggaaaccggcttcatgcgcgggcgcgccgaacacgcgggtaatcgcaattattttggcgaagagcgcgtcagcaacgcaccgcagagcccgatcttcggcccgctgcagcgcatcgcgaccttgcggcgcaataccccggccctgcagcgcggcgtgcaggtggatctgcagttgcgcggcaaccaggcagcgttcctgcgcgtctaccagcatgcgggcatgacgcagaccgcgttggtcttgctcaacaagggcgacgcggcggcggacattgcggtgtcccgcttgctgcagcccggcagctggcgcgacgccttcagtggcgagcaggtgcaggtgcaaggtcgcgtgacgctgcaggtgccggcgcatggcgtgcgcgtgctgctgtcggatgcgccggtgaccgacgtcgccctacgcaagcagctggacgcacagatggcggaccaggccgcgcgcgatgcacgcaacaagtaggagcgcgccagcgcgcgatgcggcgttatcggtgacgcctcatcgcgcacaagtgcgctcctacacaggcatcagtcggctgcaatagcgtcagcaggtcgcgaccaacaacgatggcgactcatccgcgcgacgcatgctccaacgattcccgcttcccgcttcccgcttcccgcttcccgcttcccgattcccgaatctcagcgcgtcgcgtttccgctcgccgccttcccgcgcagcccatcgctggactccgcagccgtcgacgggttccaccccagcggcggttgcaggccactgctggtcgacgactcgcgcaccgccagccgaaccgggattgtgcggctctgtgcagcgtcaccacgaatcagcgccaccaggctttcgaccaacagggcgccggcctgcttggtgtcctgttgcacggtcgatagtgctggcgccaccgaggcagccagggcgatgtcgtcgaagccgctgacggccacgtcctgcggcacgcgcagtccgcgttcgcgcagtgcccgcatcgcgccgatcgcaatcaaatcgctggccgcgcacaccgcgtcgaacgcctgcccgctgtcgagcagcgccaggcacgcggtgtaaccggacagctcggtggtaatcgcatcgaactgcagcgcggcatccgccgccacaccctgtaacgctaacgctgcgacatgcccgcgatagcgctcctcgaactccgggtaatggttggaggcgtggccgagaaatgcgatccgccggcagccctgcgcgagcaggtgctcggtgatgtccagcccgccctgatagttatcgctgccgatcgaaatacccggttgacccggcagcgccgcgccccagcgtacgaagtgcgtgccttgttcgaccagcagctgcagccgctggcgtgattcctgataatcgccgtagccgagcaggatgatgccgtcggccttgttgctgtcttcgtaatcggcctgccaatccttggacagctgct

SEQ ID NO: 20 - DNA, 2957 bp, Xanthomonas campestris
  CDS (1001)..(1957)
cactgcacgagatcccgaccttgcggcggtttgcccagctcggtggcttggacaacgttccggacgagaccacgattctcaactttcgccgcctgctggagacccatggccttgccgcgcggatgctggaagctgtcaacgcgcatctggcgcgcaagggccagagcctgcggtcgggcacgatcgtcgatgcgacgctgatcgcggcgcccagttcgaccaagaatgccgaccatgcacgcgaccccgagatgcgtcagaccaagaagggcaatcagtggtatttcgggatgaaggcccacatcggcgtggatgaattttccgggttggtgcaccacgtccattgcacagccgccaacgtcgccgacgtcacggtgacgcacacgttgctgcatggcaaagaagacagcgtgttcggcgacagcggctacaccggtgcggataagcgcgaagaactgcaggactgcgaggctgcatttttcattgctgccaagcgttcggtgcttcaagccatcggcaacaagcgggagcgtgctcgggaacagcgttgggaacacttcaaggccagcgtgcgcgcgaaggtggagcatccgttccgggtgatcaagcgccagttcggttacaccaaggtccgctatcgcggcctggcgaagaacaccgcgcaggtgctgacgctgtttgcgctgtcaaacctgtggatgaagcgaaagcagttgatgcctgctatggggagcgtgcgcctgtaacgcgggcaacaccctgcaaacgcgccggaaacggcaaaaaatcgaggatctgagcgccgtcggcctggccgatgtggttaggctcatcctccgacgccgttgatcagaccatccctaaagcggccaggaagcagatacgcagacgccagacaggcgcgagcgtggagatgcatcggtatggactgagtgtccacgccaacggctccgcgcatgcgcctgcctggcgtttgctcgctacatggtgcttggggcgcttacttgccggcgcgacgcgcgtgctcgctcaggatcgacatctgcggcttctcgctgccgtccttgcccggctgcacattgaacgggtagtccggcttccaccacgcgccggccgcccaccaggtccagcccagccacacgtcgctgttcttttccatgtagctcagcatgcctttcagcgcgtcgttgcagaccgcattgttggccgtgccgaactcgcccaggaagcctttctgcttgttctcgcgcagccagtcggtaaagccgcgcaacttgtctgcgcccaccgtggcactggtgcattcgggcttggtgccgctgtagtcgccgtccaggtattgatgcgcttcgaaggccagattgttgccggggtccttgagaatctgcagggccttggcattggagacgccgtagctggtgctgcgccagctgtgcgcgccggtgtaggccgtgccaggcaccagaatcaaattgttcgcgccggtcttgcggatggtgttgatcgccgcttgtgcggccgcggcccagtcggtggaggagatgccattgggctcgttcatcagcccgaagatcacctgcttgtcgtctttgaactccagcgccaggcggcgccacagatcggccagtgctgcaggcggcacctcgtcggtgccgatgcgctttccgttgtacttggcgtagttgtgcaggtccaggatcaggaactgcttgttggccttggcggcggccacggattttttgatcaacgccagttgcgccgcatccagctcgccattgagggttggctgcaggcgctcccacaggaacggcaagcgaatggtgttcatgcccttgctggcgaaatagctgaaatccgccgccgccgggtaggtgtagtccttgtagagcgtgccgggcttcttgcgcgagttgaactcggcgccggagatgttgacgcccacgtacttcaatccgccgcgggcgttgtctgcctccttggcatgcgccaacggcaacgtggcgagcagggccagcgccagtgcggcagcgtgggtgcgggtggagcgggggagagatgggaacatgggggagcctccttcggcagtcgacgtaggcggccgcagggggagggcacggccagcgaggatggtgcagcgccacaccatcgatggtcagcctaggggcctcggcgttagcgcgccattcacgcgggcaacgctgatttgatgatgcgcgaacgtccaaagcgccatgctggcgccacggtcgagttcgcaagccgactggcaagacgcactgtcgcaggagtgcgacggagcggtgtcctaagctaacgcacctacttttttgctgtctgcgccctgcgcgggcggcgtttccgtcccctggagtattcccatgagcaaagcccacgcctacgccgcccaaaccgccgaccagccgctggcgccgttcgtcttcgaacgtcgcgcgcccggtcccgacgatgtgcagatcgacatcgcctactgcggtgtgtgccactcggacctgcacaccgcacgcaacgaatggcacaacacggtgtatccctcggtgccgggccacgagatcgtgggccgcgtcactgccgtcggcagcgccgtgaccaacttcaaggtcggtgacctggccggtgtgggttgcatggtcgacagctgccgcagctgctcctcctgccaggaaggcgaggagcaatattgcgaacagggcttcaccggcacctacaacggcccgatgtttggcggcggcgagaacacctatggcgggtactccgaccatatcgtggtggaccagaagtacgtgctgcacatttcccacagcgacaacctggccgccgtggccccgttgttgtgcgcgggcatcaccacctattcgccactggcgcattggaaggttggccccgggcagaaggtgggcgtggtcggcctgggtggcctgggccacatggcggtgaag

SEQ ID NO: 21 - DNA, 3668 bp, Xanthomonas campestris
  CDS (1001)..(2668)
tgtggacagcgattcgattccgttcgacggcaacatcggcgtgcgcgtggtgcgcaccgaagtcggctccaacggcgtgcggaccggtaccgatgccgagggcggtggcctgattccggtggctgcgcagcagacctacaccgacgtgctgccgagcttgaacctgcgctggatgctgagcgaccagctgcagtggcgttttgcggcctcgcgtggtatttcgcgtccaaccttcgaccgtctcaacccgaacctgagcctgagcaccggcacctcgaacggtgcttcgaccttcaccggcaccgcgggtaacccgagcctggaagcggtcaaggctgaccagttcgacaccgcgctggagtggtatttcggccagggctcgatgatgtacggcaccctgttctacaagaaggttgaaggcttcatcgccaacgccgtcttcaacgaggtgtacgacggccaggtgtggcagatcacccgtccggtcaacggcgatgccggcaagatccgcggggcggaactgggttacacgcagttcttcgacttcctgccgggctggctgagcggcttcggcctgcagaccaactacacctacgtcgacagcgaggcgccgagcccgacggccaccgacaccaacggccagtcgctgacggtgccgctggaaggcctgtccaagaacagctacaacgcgatcctcagctacgaaacggcgcgcttccagggccgtgtggcctacaactggcgcagcgactggctggtgaccaccgcgggcaacggcactgaaaatctgccggtctacaacaagggcttcggccagctggatgcctcgctgcgtttcaacatcaatgatgtgtggtcgatttcgctggacggcgtgaatctgctcgatacccgtcgcgaaagctatctcgggaccgagtcgcgctaccgcgactttgtgatcaacgatcgtcggtatggcctgactttgcgggccagcctgtagaacgcgccgtaggaagtgcgtcgccggtccggcgagctgtgcaggcagctcgttggactggtgtttttcctatcgacggcgttgccgtcgatcgcgaggggagccggaggaatgatcgcaatgatcgatccgcctactaagcgccgcaccaccacaacaggagcatggctggctgtcttgagcctgctgctgttgctcttttcgacgcccagcgtgcgtgcggccgagcccgccacgtccgggccctaccagtggcgcagtgtcgccattggcggtggcggctttgtcaccggtgtgctgtttcatcccgccgaacgtggtctggcctatgcgcgcaccgatgtgggtggcgcgtaccgctgggatgcgcaggcgcagcagtggaccgcgctgaccgactggttgggcgctgacgactggaacctgatgggcatcgacgcattcgccgtcgaccccgccgatgccgatgcgctgtatctggcggccggcacctatatgcatgaacgcgccggcactgccgcagtgttgcgctcgttcaaccgcggccgcacgttcgagcgtgccgacctgccgtttaagctgggtggtaaccagctgggccgcgccaatggcgagcggctggcggtggacccgcacgatggccgcgtgctgctgctgggctcgcgcgatgccggcctgtggcgtagcgacgatcgcggcgcgcactgggcgaaggtggcgtcgtttccggacgccgcgctggccggtgccaccgcgcgcaatcatgttgggcgcgagcaggcggtggggatcgcctttgtcgtgttcgacgcagccagtggcaacaacggctcgccaacaccgcgcatctacgtgggcgtgtccaccgaacagaccagcctgtatgtgtccgaagatgccggccgcagttgggcaccggtggccgggcaaccacgcggcctgcgcccgagccacatggccggcggcagcgatgggcactggtatctgagctatggcgaccagcccgggccggacctgatggccgggggagccttgtggaaattcacgccggcacaggggcgctggcgtgagatcagcccgattccgcagccagccagtggcgatggattcggctggggtgcggtggcggtggatccgcaacatccgcaggtgctgctggccagcaccttccgccgtcgcacgccgcgcgacgagctgtatcgcagcgtggatggtggcaagcactggacgccgttgttggccgatgcggtgttcgatcacagcgccgcgccgtggaccgcacatgccacgccgcactggatgggggcgctggcgatcgatccgttcgacggcaaccatgcgctgttcgtgaccggctacggcatctgggcctcgcgcaatctgcaggatttcgccgcaccgcagcgcccgctgcagtggtggttccaggaccgtgggctggaagaaaccgtgccgctggacctgctcagcccgatggctggcgcgcatctgctcagcgcgctcggcgatatcgacggcttccggcatgacgacctggaccgcgtgcagttgcagtacgccggcccacggctgaccaatggcgaaagcatcgatgccgccggccaggcgccgcagtgggtggtgcgcagcggtaccgtgcgcgcccgccgcaacaacgaaatccgcgcgctgtattacgcgtgatggcggcaagcaatggactgcgtttgcgagtgagccgccagccgggcagggcgccggcagcattgccattggtgccgatgcggcgcaggtggtgtgggcaccggcgcgcggcggcaattggcgcacgtccgacttcggcgcgcagtggcagcgtgtgaacgccctgccgaaccccgcgtgggtggtgaccgatcgggtggacgcacggcgctggtatgcggtggatgtcgccagcgggcagctgtacgagagcaccgatgccgcgcgcagtttccgtgcgaccggtgtacaggtgggcagcccggcgcgcgatgagcgcacccggccgcagctgcgtccggacccgtggcgcgcaggcgtggtgtatctggccagccccggcaagggtgtgatgcgctggcaggacggtgcgctgcaggtgctctcacagccggacgaagcacgctcgctgggcatcggcaaggcgctgcgggccggtgcgccgccggcgctgtatctggccgggcgcgtgcagggcgtggatggcgtgttccgctccgacgatggcggcgcgcagtggcagcgcatcaacgatgacgcgcaccgcttcgggcgcccgtacagcgtgaccggtgatccgcgcattgccgggcgtgtgtacttcgccaccggcggccgcggcattttctacggcgatccgagatgagcagtgcatgcctgtccgcgacgtcctgctgctttaccgtgtacggcgccgccggcctggccgctgcgggcgccgtgacaccgccgaccctggagatcaccgcatgaccccgtcccacatttcccgcgcctgcttgctgggtgtgttgctggccagcggcagcgccgccgcgcaaccgagcctgccgttgctgtttgccgatggcgcggtgctgcagcgcgatgaggcaatgccggtatggggctgggccacgcccggcgccgcaatcacggtgagtttcgatggcaagcaggccaccgccaagaccgatgcgca

SEQ ID NO: 22 - DNA, 3749 bp, Xanthomonas campestris
  CDS (1001)..(2749)
gatcggcatgccgccgaccgcggcacccggcgggcatatccatgcccttgtgtggggtaagggaccgggcgaatccgacggcgccagcaagtccatcgccaacaaccagggcaagggcttcgacagatactgcgacccgacctatacgacgccggacggcacgttgacgggtgccttgccgaacgcgccgatcgccggtaactggttccacgcgcagttcctccagttggtcgccaacgcgtatcccgccatcggcacctcgaccaaggcggcgctgcagtcggcgtcgaccgacgcggtgcccgccagtcggccgacggcgaccaaggggttgaccgccaatgcagcggatggcgaggtcaggctcagctggtcgcccgttgccggtgccacgggttacacggtgcaacgtgtcgccgacgcaaccgctgctcccatcaccgtcgcatcgggcctgacctcgccctcatatgtggaccagacgctcaccaacggcaccacgtactactacaaggtaacggccaacggcgcatcgggtgcagacgcgagctcggtcaccgtgagcgcgacaccgcatcggtgatacgataactgcgttgccacagagcggtagaggctagtgtgctctaccgctcctcggccgcgggaggagcgctgcaaccacgatggcggcgccttgcgcccgccatttcaatcgctgtcgtgctgctctacctctgtctggagcgcggcatgcgcttcgaatgagatgacatcggagcagatgccgtcgctggcggaacgggcgctgcatggcacaggctcggtagcgccacggcaagcggcgcagcgtccactccgtgccgctgacggccaacccggcctgtggcatgccgggactgcatcgcgcgcgttgccggggagctgcgcccttttgtctccacccgcacaggccgcttcgcacgcaaccgtacataacacactccagaagacgtccatgccatcacatatccatttcttgatcgcgctcgcaacgctcatcccggtcaccgcgccggcgatgcaggtcagtacgcaagctccccttgtcgatgcaacaggtcagacgctgcacattcgcggggtcacctggcccggcttcgatcgcgccgggttggcggccgtcggcatgcgcaacaacacactggcgcaactgctggatcggatgcaggcgtcagacatcaatgccgtacgtgtgccggtctgcgccgctgttctgcaacgcgcccctgtggcagctgcagaagttgccggcgattccacgctgcgcggattagattccttgcaactgctcgacgcagtggtgcatgccgcaagccagcgcgggatgcaggtgatgttcgcgtttgcagatggaggctgcgatgaccgcgcaccgctcctgggcgcacagcagcaagcatggacgcgcggcctggttacgctggcacgccgctatggcggcaacgccaatgtcctgggcattgaccttggcagcagcggctatcgcaatgccagctgggcaggaaacgcggcggatcaggattggaaccgggtagcgtcgcgcgcagttgcaagggtgctggcgcaggcaccgcgctgggtcgtcggtgtggaaggtgtgggcagcaatgcggtgtgcagtgacccagagcgcaaggcgcctggcagcaatcttcagccgttcgcgtgcgtgccgctggacatcgctcgccgacacttggtgttgatgccgaagttggcgggcccggatcgcgatacgaccgatgctttcgccgcacccggatttgcgcaggcactgcccgccatgtggcaacgcgacttcggccaattcgcaatcgaccatgcagtggtacccgtcagcctcggcggtggactgggcgatggcgacccgcgtgacccggcatggcaaaccgcattgagcggttatttggcaaacgcaggaatacgcagcgcatttctcggcagttgggaaacgggcaatgccaacaacggcggcctgttggcgccggatggcagcccccgcgcagacaaactgctgatcttgcgtcatgcctggggcaggttacccgtgatgcccgccattgcgacagcaaccggcgacagcacgaagaatgcgagtgggaagaagccgtggaatagtactttcaccggcaccgccaccgtgaccggctccggctactctggcggcgcgctcctgctggaccccattccgagtgatgccttcatcaccgcgctcaacccggtgcaactgaacttcggtggcgtcaaggccgcgcttgccggcgcgtacctgcaggtgaacggccccaagggcaccaccacggtctacgtgaccgacctgtatccggagggcgcatccggcggcctggatctatcgcacaacgcgttcgcggcgatcggcgacatggtgcaggggcgcatcccgatcagctggaaagtggtacgcgcgccggtcaccggcaacctgcaataccgcatcaaggaaggcagctcacgctggtgggcggcgatccaggtgcgcaaccacgcctatccggtggtgaagctggaggtgaagcaaggcagcacctggaagaacctgcagaagatggactacaaccacttcctgggcgagcaactgggcaaccaaccgctaaccctgcgcatcaccgacattcgcggcaaggtcctcaccgataccttgccacgtctgccagaagacggcagcaagccggcgtatttcgaaccggggcatgtgcagtttccgtagcgcgaacaggtggtttgcctgaactgagcgttggattggttaacgggcgtggacttgtttagatcgtttgcttgtctagagcgacaagcgctttcagcgccgctagttgatctcgctgggtcagcccggtgcgtgcgagtcgctgtcacgcaccggctgtacgcaccggccgtcgccacgttggtgcccgtgcaactgcttcacacacgcagcgccagagacacagtcatcagcttcagcgagccagccttacgcagttgcgcgcgtgtggccaacgcttggccgctcgagtagagcaccgtgaatcgccagcaataccgaccaaatagcggtatacgctaattggcgctgttgtaaacggcaatgaggccatgctgttattaccatgcgcctctggcagcgaacgacggcgcggcttctatgcaacgtcgcatcggcctctacttgggtagtttcgccggtgcaatccaggtgcatcaaccacccgccaggccaccaccgacgcgcccgggtaatgcctgatcgtcgtcgtcctcggggttgtcgagatcgcccacttcatcgtcgataccgccgcgccgtggcgaggggtcgttcgagggcggcaagtcgtcgtcgcgttgcggccccggcgtgccgtgcaagcgaaactgcgagacaacggacgcaggcggatcggtgttgagcgaatacaggatcatgcgtcatctccggctgggaacctgtgctcgacgctagtgcgcgcagcgctagcaggccgtgagccgcagcgcgtgcgcgctgacatgccgttgacgctgagcgcgcaccgctgcgctgtggttgcctgcgggccagtggctatcatggggctccgccctccaggccgccgtcatgcgcccacgtcttctgatcatgctgctgctcctgtgcggcgccgctacggcagcagcaccggcctcgcgcctggatgcggtggtgcgcgccggcgtgctgcgcgtctgcaccaccggcgactacc

SEQ ID NO: 23 - DNA, 3668 bp, Xanthomonas campestris
  CDS (1001)..(2668)
cagctcagtctgccggtacctgctcacgccatctcgaccgtagcgacctcgcatcgaaccatcccgaggtcttccgacaccggcatccaggtcagacgcgctggttcgcgttcactgccaggcaccgggaatcgcaagctgtggcggaatgccccggaaccacgacgccaaccgagactgacatccaacataatccagggaaatatacttttccgccccactcctaaagtcgaaaataaaacatttctggcagcattaaataactttcgatattatctttatgaatcaactctccgaataaacgatatccgtctgcgtctgcgttggcgttggcgttggcgttggcgtctccacataatcggcatctgcaataactccacacctttcgcagatggtgagcgtagtcgtccacagttccaggatctgctttattgtagagttcgctttctagaggccgcgcacggattcgcgccatcgatgctggccttctacgttcttcgcctcgccagacagtctcccgtgcatctccagccacaccttgcctgcaaggcctgctgccgttgtcgatcaatacgccggccatgccggtcgatggtcgcggcaggcgggtcgacgaaagcgggcatgagccgcaacgacgcggcacacaatcgcccggtgcgcctgggcgtcagcccgctgcagctgcgctcaaacgggcaacggaatcgacgcattgcgccgaccgatgcgcgaatacggcgagcagcttgaatgcgacataccgcaccatctcctgcgccgatcgcccccggcgcaggaccaccgatgcacgcgcatctggggcgcgttcggttgcgcacgtgcatcactcaccccgagcgggcatccatcgagagaaaccaccagtcacggagaatcacgcgacatcttgatatttcactaattacacaccgtttcaccatttcccatctcaaggcgaaattcaatgtcatcttcgacaaacctccgaccctgcgtcacatccaagctcgccgtttcgttgctgacaggcgcgctgctcgttcccgttgccgcatccgcccagagccatgtcgacaacccgtttgtcggcgcgagcgggtatctcaatcccgattattcgaaggaagtcaactcgtcgatcgtcaaggtcaaggatgtgcaactgaaagccaagatgcaggtcgtcaagtcctatccgacctccgtctggctggattcgatcaacgcgatctatggcggctcgcgcaacgccggccgcctaagtctgcaaggccacctggatgcggcattggcgcagaaaaaggccaatacgcctatcacggtcggctttgtcatttacgacatgccggggcgcgactgccatgcgctggcctccaacggcgagctaccactgacgcaagccggcttgcagcgctacaagaccgaatacatcgacgtcatcgcatcgacgttggccaatccgaaatacaaagatcttcggatcgtcaacatcatcgagccggacagccttccgaatcttgtcaccaatcaaagcacgcccgcctgcggccaggcggcatccagcggcatttacgaggccgccatcaagtacgcgctcgacaagctccatgccactcccaatctttacaattacctggatatcggccattccggctggctggggtgggatagcaaccgtagcccggcaatttcgctatacacacgggtcgtgcagggaacggcagccggtttggccagtgcggatggcttcatcaccaacaccgccaattacacgccgcttcatgagccgaacctgcccaatccggacctgacgatcggtggccagcccattcgctcgtccaacttctatcaatggaacagtttcttcgacgaatcgacctatgcggaagcgctctacaacggcttcgtcggcgcgggttggtccagcaagatcggctttctgatcgacaccggtcgcaacggctggggcggcaaagcgcgccctgcatctgccagcggcaatgacatcaacacctacgtggactccggacgcgtggaccgtcgcctccatcgcggcaactggtgcaatcagagtggtgcggggatcggcatgccgccgaccgcggcacccggcgggcatatccatgcccttgtgtggggtaagggaccgggcgaatccgacggcgccagcaagtccatcgccaacaaccagggcaagggcttcgacagatactgcgacccgacctatacgacgccggacggcacgttgacgggtgccttgccgaacgcgccgatcgccggtaactggttccacgcgcagttcctccagttggtcgccaacgcgtatcccgccatcggcacctcgaccaaggcggcgctgcagtcggcgtcgaccgacgcggtgcccgccagtcggccgacggcgaccaaggggttgaccgccaatgcagcggatggcgaggtcaggctcagctggtcgcccgttgccggtgccacgggttacacggtgcaacgtgtcgccgacgcaaccgctgctcccatcaccgtcgcatcgggcctgacctcgccctcatatgtggaccagacgctcaccaacggcaccacgtactactacaaggtaacggccaacggcgcatcgggtgcagacgcgagctcggtcaccgtgagcgcgacaccgcatcggtgatacgataactgcgttgccacagagcggtagaggctagtgtgctctaccgctcctcggccgcgggaggagcgctgcaaccacgatggcggcgccttgcgcccgccatttcaatcgctgtcgtgctgctctacctctgtctggagcgcggcatgcgcttcgaatgagatgacatcggagcagatgccgtcgctggcggaacgggcgctgcatggcacaggctcggtagcgccacggcaagcggcgcagcgtccactccgtgccgctgacggccaacccggcctgtggcatgccgggactgcatcgcgcgcgttgccggggagctgcgcccttttgtctccacccgcacaggccgcttcgcacgcaaccgtacataacacactccagaagacgtccatgccatcacatatccatttcttgatcgcgctcgcaacgctcatcccggtcaccgcgccggcgatgcaggtcagtacgcaagctccccttgtcgatgcaacaggtcagacgctgcacattcgcggggtcacctggcccggcttcgatcgcgccgggttggcggccgtcggcatgcgcaacaacacactggcgcaactgctggatcggatgcaggcgtcagacatcaatgccgtacgtgtgccggtctgcgccgctgttctgcaacgcgcccctgtggcagctgcagaagttgccggcgattccacgctgcgcggattagattccttgcaactgctcgacgcagtggtgcatgccgcaagccagcgcgggatgcaggtgatgttcgcgtttgcagatggaggctgcgatgaccgcgcaccgctcctgggcgcacagcagcaagcatggacgcgcggcctggttacgctggcacgccgctatggcggcaacgccaatgtcctgggcattgaccttggcagcagcggctatcgcaatgccagctgggcaggaaacgcggcggatcaggattggaaccgggtagcgtcgcgcgcagttgcaagggtgctgg

SEQ ID NO: 24 - DNA, 3268 bp, Xanthomonas campestris
  CDS (1001)..(2608)
  unsure (1)..(3268): unsure at all n locations
agcagcaccatcaggatcagcaggaacacgccggcaaacagcgtcgacagccggccgcggccgccgtatttcacgttgcccacggtctgcccgatcatgccgcaaccggcgatgccgccgaacaggctggcggccgcattggccaggcccaggccggtgcattcgcggttcttgttgctgggcgtgtcggtgagttcatcgaccacgcgcgcggtcatcatcgattcgagcaggcccaccattgcgatggccagcgccggcagcgcgatgatgcgcaaggtctccagcgtcaacggcacgccgggccattgcaggaacggcagcgcggtgggcagcttgcccaggtcggccaccgtcttcagcggcaggtgcagcgcgctgctggccaccgtgagcagcaggatgcacagcagcggcgacggaatggctgagacaccgggcagccgcaaccgcggcaggccgtagatgatggccaggcccacgcccagcgtgacccaggtggtggggttggcaccgagcaggtgcggcagctgcgcggcgaagatcagcaccgccagcgcgttgacgaacccggtccgcaccgagctgctgacaaagcgcatcagcacgcccaggcgcagcagcccgaacacgatctgcaccagcccggcgagcagcccggcggcgagcagatacggcaacccatgcgtggccaccagcggcgcagccaccagcgccaccgagccggcggctgcggaaatcatcgccggccgtccgccacaaaacgcgatcacgatgccgatcacaaacgacgcgaacaagccgacctgcgggtccacgcctgcgacgaacgcaaaggcgatgacttcgggaatcagggcgaacgtggcaacggcgccagccatcagttcgcgcgcaggcgaggcgcgccattgcgccagttcggtgcgcaggaaggacatgggggacactccagggacaagaacgacagtcctgcggacagcgcgcagggggcactagtgtgcgggaaagcgaccgctcccgcagccgcgatgtgatcggtgcggcaatggtgttttctgtggggacgatcacaccacgcgacgcgcgcacagaccaagatgcccgccttaccgcgctcgggtgtcgagcccggttctctagggagatcaccatgtccatattcaggaccgcaagcacgctcgctttggccaccgccctcgcactggccgccgggccggccttcagctattccatcaacaacagcaggcagatcgtcgacgacagcggcaaggtcgtgcagctcaagggtgtgaacgtgttcggcttcgaaaccggcaaccacgtgatgcatggcctgtgggcacgcaactggaaggacatgatcgtgcagatgcagggcctgggcttcaacgccgtgcgcctgccgttctgcccggccacgctgcgtagcgacaccatgccggccagcatcgactacagccgcaacgccgacctgcagggcctgacctcgctgcagatcctcgacaaggtgatcgccgaattcaatgcgcgcggcatgtatgtgctgctggatcaccacacccccgattgcgccggcatttccgagctctggtacaccggctcctataccgaggcacagtggctggccgacctgcgctttgtggccaaccgctacaagaacgtgccgtatgtactcggcctggatctgaagaacgaaccgcacggcgccgccacctggggtaccggcaacgccgccaccgattggaacaaggctgccgagcgcggctcggccgcggtgttggcggtcgcgccgaaatggctgatcgcggtggaaggcatcaccgacaacccggtgtgctccaccaacggcggcatcttctggggcggcaacctgcagccgctggcctgcaccccgctcaacatcccggccaaccgcctgctgctggccccgcacgtgtacggcccggacgtgttcgtgcagtcgtacttcaacgacagcaacttccccaacaacatgcccgccatctgggaacgccatttcggtcagttcgccggcacgcatgcgctgttgctgggcgagttcgatggcaagtacggcgaaggcgacgcacgcgacaagacctggcaggacgcgctggtgaagtacctgcgcagcaagggcatcaaccagggcttctactggtcgtggaatcccaacagcggcgacaccggcggcatcctgcgcgatgactggaccagcgtgcgccaggacaagatgaccctgctgcgcacgctgtggggcaccgccggcaataccacgccgacgccgactcccacacctacgcccacaccgacaccgacgcctacccccacgccgacgcccaccccgggcaccagcaccttcagcaccaaggtgatcgtggacaacagctggaacggcggctattgcaaccgcgtgcaggtgaccaacaccggcaccgccagcggcacctggtcgatcgcggtgccggtcaccggtacggtcaacaacgcctggaatgcgacctggtcgcagagcggcagcacgctcagagccagcggcgtggacttcaaccgcaccctggcagccggcgccaccgccgagttcggcttctgcgccgcgagctgagtgcactgtggcgggtacggctcccgtgtccgctacctttgcaacatgcagtggcattgtggaggcgctgcgtagcaggttgatgatcacctgcggcgcctaacccacgacctcgtaggagctttacctcattctcacggaaaccataccctatcatcccaactagcacccccacctcttcatcctcccaatctccccccttcacgacatacatcaataaaaatacgatcggcatcatcaccctacctctccattcccagcacctaaacaaaactccactcgctccaaccatccacaatccctcgtaccacgacgctccacatacataataaacatcctaactctacggtagtccacatagactctcacgtcatctcagctaactaccaattccttctgcccctcaagtatatactattctcaccaccacagttctaccctgtcatcgacaaatacgattcagatcatgcgccttccgccaggcacacacgagtaccacctccccagaaatnaataacgctactccacaaccacgtaacaagattctagcacatatacantccatagaacaacctcacgagcaactatattcgaatctgcgttctacttacatgcactacggcatacataatcttcaggtttcgcatgtcacctacgtcc

SEQ ID NO: 25 - DNA, 3002 bp, Xanthomonas campestris
  CDS (1001)..(2002)
ttgcgcgccagatgcgcgttgacagcttccagcatccgcgcggcaaggccatgggtctccagcaggcggcgaaagttgagaatcgtggtctcgtccggaacgttgtccaagccaccgagctgggcaaaccgccgcaaggtcgggatctcgtgcagtgcttcttccatcgccggatcgctcaacgcataccactgctgcagcaaatgaatccgcaacatcgtcgccagtgcgtacggctgccgacctggccggcccgacacgggatagtgcggcgcgaccagcccgagcagttgctgccacgggacgacctgctccatctcggccaggaagatctcccggcgggtctgcttgcgcttgcccaggccctcagcgtcaccgaacgtcagttgcatggatcactcctcaacatcaggcgggtagtgtcgcgtatttgtggtgcgttgttcagaggttccttagcgcagccaactgcgaacgatgcacgagtgacgctggcgatgtctgccacctcgcgcgccatttctgcagcgcgtttgtggaactgccacgtgcgcgtgcatcggctcgcagccacataggcaaccgtgatcacgcgggcgcaccgcggtcgttgcggtgtggctgcccatgctgcagtgatcccccgcatgcagtgtttctaggcaggcagttttttcgccacaggtctcttggccgacgcgccacgcgcgtcgttgcaagctgcgcgcacgcgtccatacatgggcatgacaaagtgcctctgtcgcggatgcgtcgaccagatcctgccgccactgcgaaacaccgcacagcttggaagagtgatacctcgcatctgtagtttcagcgtgccgttgcgcctagggtagagacagatgcgctgttggccgcgtcgcgcggcatgacgttgcgggcagcgtatctcccccgcaactgcaaggaacaccccatgtctgcctccccctcctggccacgccagctgctgcgctgcgcgttggtcatgctgtgcatcgccacgatgtccatcgcacaggcgcaaagccccaatcgtctgaaatacgcaggcgtcaacatctccggtgcggagatccagtcgtccaaaaaaccgggcgtgctcaatatcgactaccgctatccaaccgcgtccgagtacaggtacttcgcgggcaagcaaatgaacatcgtgcgcctgccgatcctgtgggagcgcatgcagcccaaagctcaaggtccgctggatcaggcacaactggcattgctgaagcaggcagtggccaacgccaaggccgccaatcaatacctgattatcgatgtgcacaactacgccaagtactacggccagaagattggcagtaagcgggtgccggtgcgcacgttcaccgatctctggcgccgtctggccatcgcgttcaagagcgacaacgccgtgattttcgggctgatgaacgagccgtacgacatctcgccagagagttgggccgccgccgcgcaggcgtccatcgacagcattcgtgccaccggtgccaccaacctgattctggtgcccggtgcattgtggtcgggcgcacacagctggtattccaccgtggccgggcagtccaatgcagtggcgcttgcgaacatccgcgacccattgaatcgctatgcgatcgaagtccatcaatacctggacaccgactccagcggcaccagtgccggttgtgtgagccgcacgatcggtgcggagcgcttgcgcagtttcaccggctggctgcgcgcgcaaggcaagcgcggttttcttggcgaattcggcaccgccaataacgcaacctgcactgcggcgctggacggcatgctcggttatctcgaaaccaaccacgatgtgtggatcggttggacgttctgggcggcaggcgcgtggtggaacaccagctatccgttcaatgtgcagcccgatgcgcaaggccgcgacaagccgcagatgaaaaccttgagcgcacgcgcgcatcgcgtcacccgataacgggcatcgatcagttcgcttgtgtacaactgcagcagccgacgcacgcagtagacctgtactgctggtgcgtcggttgcatgcgtagccggagatttggcgtcacgctttcgtcgcctagcaaatcgcatgtcgagatttcgtcgcggtcgcgtcacgcgtgaaagcgcaatctcccttgaaagcaggcacaaccgcacgaacccggtgccagctcctcccacgccgcataaagtgcaggccacaacgcagtttgtttgtgagctcctgcgcacatcatctttaaccaccgcgcgatgcgcggctggcacgcgtgatgcattgaacgggcagatgcagcgtaacgaacgtcgatcgcacgccatggggctgcgatgcatcttttccgtcgtcgaaggagtggtttccatgtcgtgttctctgtggttgcgcaggctaatgtcctgcgcggtgttcttcctgctgctcggtgttctgccgatgggccatgcacaaacgcgtgcactcaagtacgccggcgtcaatttggcgggtgcggaatttgcgtcatcgaaaaaacctggcgtcctcaacaaggactacatgtatcccgccagcaccgattacagctatttcgctgggactggaatgaacgtgattcgcctgccgatcctgtgggaacgtctgcaacctgccgcacgcggcgagctggatccggcgcagctggcactggtgcagcaagcggtggcacgcgccaaggcatcgggcatgtatctggtgctggatatccacaactacagcaagtactacggttataaaatgggtgggccggaagtgccgctcgccacctttgccgatctgtggcggcgcctggcggtgatcttcaacagcgataacgccgtgatcttcgggctgatgaatgagcccaacaacatctctgccagcgaatgggccggtgcagcgcaggcaggcatcgatgcgatccgtgccaccggcgccaacaatctgatcctggtaccgg

SEQ ID NO: 26 - DNA, 2040 bp, Xanthomonas campestris
  CDS (1001)..(2008)
gtacgacatctcgccagagagttgggccgccgccgcgcaggcgtccatcgacagcattcgtgccaccggtgccaccaacctgattctggtgcccggtgcattgtggtcgggcgcacacagctggtattccaccgtggccgggcagtccaatgcagtggcgcttgcgaacatccgcgacccattgaatcgctatgcgatcgaagtccatcaatacctggacaccgactccagcggcaccagtgccggttgtgtgagccgcacgatcggtgcggagcgcttgcgcagtttcaccggctggctgcgcgcgcaaggcaagcgcggttttcttggcgaattcggcaccgccaataacgcaacctgcactgcggcgctggacggcatgctcggttatctcgaaaccaaccacgatgtgtggatcggttggacgttctgggcggcaggcgcgtggtggaacaccagctatccgttcaatgtgcagcccgatgcgcaaggccgcgacaagccgcagatgaaaaccttgagcgcacgcgcgcatcgcgtcacccgataacgggcatcgatcagttcgcttgtgtacaactgcagcagccgacgcacgcagtagacctgtactgctggtgcgtcggttgcatgcgtagccggagatttggcgtcacgctttcgtcgcctagcaaatcgcatgtcgagatttcgtcgcggtcgcgtcacgcgtgaaagcgcaatctcccttgaaagcaggcacaaccgcacgaacccggtgccagctcctcccacgccgcataaagtgcaggccacaacgcagtttgtttgtgagctcctgcgcacatcatctttaaccaccgcgcgatgcgcggctggcacgcgtgatgcattgaacgggcagatgcagcgtaacgaacgtcgatcgcacgccatggggctgcgatgcatcttttccgtcgtcgaaggagtggtttccatgtcgtgttctctgtggttgcgcaggctaatgtcctgcgcggtgttcttcctgctgctcggtgttctgccgatgggccatgcacaaacgcgtgcactcaagtacgccggcgtcaatttggcgggtgcggaatttgcgtcatcgaaaaaacctggcgtcctcaacaaggactacatgtatcccgccagcaccgattacagctatttcgctgggactggaatgaacgtgattcgcctgccgatcctgtgggaacgtctgcaacctgccgcacgcggcgagctggatccggcgcagctggcactggtgcagcaagcggtggcacgcgccaaggcatcgggcatgtatctggtgctggatatccacaactacagcaagtactacggttataaaatgggtgggccggaagtgccgctcgccacctttgccgatctgtggcggcgcctggcggtgatcttcaacagcgataacgccgtgatcttcgggctgatgaatgagcccaacaacatctctgccagcgaatgggccggtgcagcgcaggcaggcatcgatgcgatccgtgccaccggcgccaacaatctgatcctggtaccgggcgcgctgtggaccggcgcacacagctggcactcgctgaccagcgatgggtattccaatgcaaccgcgttggcatcgatcagcgaccctctcaatcgctatgcgttcgaagtgcatcaatatctggatgccgattccagcggcacctccagtgtctgcgtcagtgaaaccatcggcgccgatcgcctgcgtgcgttcaccgaatggctacgcaccaacaacaagcgtggttttctcggtgagttcggcaccgccaacaacgccgtctgcaataccgcgctgcaaggcatgctggcctacatggaaaactacgccgatgtctggctgggctggacatggtgggcagcgggcgcatggtggaacaccagctacgcctacaacgtgcatccgaacaaggacggcaccgacaagccgcagatggtcatcctctcgccgcaggccgcgcgcgctacacgctgacacgttgtcgcatgcccgcctttcgtaggagg

SEQ ID NO: 27 - DNA, 2670 bp, Xanthomonas campestris
  CDS (1001)..(2032)
cgcattgtccggcagcgctgcggccgcgggcaaactgcggaaatgccgtggccgccctgcgtcaccgcggcggcggcgtcagggcatcgcatgcatttgttgccaacgcattgcgaacgcaaccaatgacatcgccttagcgctgcagccgtcacgctaggtcatcacgcctcgcagcaggcgcattggctggaactccatgtctcgcattctgttgatcggcggccacggcaaggtggcgcgtctgctcaccccgctgctgctggccggtggccaccacgtcaccgcgctattccgtaatcccgaccacgcaagcgatgtgatcgccgatggcgccacacccgcgctattcgatatcgaacacgccgacaccgcggcgatcgccgcgcacatcaccggccacgatgcagttgtttggtcggccggtgccggcggtggcgatgccgcacgcacctatgcggtggaccgcgatgccgcgatccgctcgatgcacgcggccgagcaggcgcgggtgcggcgctacgtgatggtgtcctacctgggcgcggggctggagcacggggtgagcccggaggatggcttcttcgcttatgcgcaggccaaggccgctgccgatgcgcacctgcgcggcaccacgctggactggaccgtgcttgggcccggccggctcaccctggacccacccagcggctgcatcgcccgcgacccgggcagcgacaccaccgagggcgtatcgcgcgccaatgtcgcgcaggtgatcgccgccaccctggccacgccggccaccatcggcaagaccgtgggattcgtggatgggcccacccccatcacgcaggcgctcgcgcagctggcatgaacgccagccggtccgcagcgcgctgccatggcaagggccgctggcagctaacgatttcctaaagtcgcaccgcgcatagtcgccggcgcagcccccactgtggagatcgctatgtctgcgtttgttgcgccccgccgcgccggcatcctgccgccgtatctgctggaccacgtggcgcaggccgcaccggagcgcgcacggcactgcgcgcagctcacccgccacatcaccgcgcagctgcgccagcgccgcgcgcaggggttgctggcgcgcgacagcgccgacgatgcgcccaccgccaccaccgacactgcggtgcagcggcatctctacgacgcccagcaaggcactgccctgcctggcgtgctggtgcgcgaggaaggcgcgccgcccaccgacgatgtcgccgtgaccgaggcttacgattacctcggcgccacccatgccttcttccagcaggtgtatgcgcgcaactcgatcgacgatgccggcatgccgctgcttggtaccgtgcactacgagcgcaactacgacaacgcgttctggaccggcgagcagatggtgttcggcgatggcgacggcgagatcttcacccgcttcaccatcgccatcgacgtggtggcccacgagctcacccacggtgtcatcgagcgcaccgccaacctgatctaccagggccagtccggtgccttgaacgaatcggtctccgacgtgttcggcgtgctcgtcaagcaatacgcgctgcgccaggacgccgcgcaggccgattggctggtgggtgccggcatgttcctgcccggcgtgcagggcgtggcgctacgctcgatgcaggcgccgggcaccgcctacgatgacccggcattgggcaaggacccgcaaccggcgcatatggacgcctacgtggacacgcaagaagacgacggcggcgtgcactacaactccggtattcccaaccgcgccttccagcgcgccgcggttgccatcggcggttacgcctgggaaaaggccgggcgcatctggtaccgcgccctcaccggtggcgcgctgtcggccagtgcggacttcgccaccttcgccgcactcaccgtgcgcgtggccagcaccgattacggcgcgggcagtgccgaggccagtgcggtggaacaggcctggcgcgacgtgggcgtgctcgcatgaccgtgcaactgccgcccttgcaaccgggtgtcgtgttgcggttggcacgcgaaggtggcgtggccgcgttcccggccatgcgccgcgagcgccagctgctgctggaagagctggacgaggtgcaacgccaacacctgggcgcgctgctggatcagtgcctggcgcatgcactccccgccccgcaagccggcggcggcgaccgccgctacttcagcatcatctgggatggcgccagcgaaccgctgcgcatccccgaagaacacgccccggcggaaatcgtacggctgtggaagcagggcacgttgtagatggcttgcgcgcgagcgcggcacgcggagacgcaaagacgccggtcaacgttgcgcgcgatcgacgcctcgtaggagcagagcttggtcaggctcaccacctcccgggtggtttcgacttccaagcgcacacttttgtactgggcatccaccctggaagtgggactcaagaaaacattttgtccgtagcactggcgctctttgatcgcaccaaatgcacccttgatcgaatccatcatgattgatgaccccatgcccccgctgcggggacgccttgcctggaaatttggtcttatggcgttcctttctagcccagactgggggagggtt

SEQ ID NO: 28 - DNA, 2870 bp, Xanthomonas campestris
  CDS (1001)..(1870)
agccggggcgtcgatgcgcgtggcagcgtccaggctggcaacgcgcggcagccgctgcggcgaaaaatgctgcacagcatgctgcagcacaccggccaccgatgcgcgcgcgggcagggccgccggtgcctggcccagcgcccgccaggccgcgcgcagcgctggcagcgggtcgctgtcatccactggttgcgccgcatgcgacttggaaagcttgcgcccgtcggcgcccaggatcagcggcagatgcagatagcgcggcggtgccacgcccagggcctgctgcagcaccaattggcgcggggtggagtcgagcaggtccgccccgcgcaccacatcggtgatgccctgcgccgcatcgtcgaccaccaccgccagctggtaggcccagtagccgtcggcacggcgcagcacgacatcgcccacctcggcgtacacatcctgggtgatgcgcccctgcagcgcgtcctggaagcttgcctgggtgttcggcggcacccgcaggcgcacggcccggcggatgcccagtggcgccacgcagacgtggtggatgccacccatcacggccagctctgcgcggctgcaactgcattcgaacgccgcaccggtgcgcagcagccgcgacagtgccgcctcgtaatgcgcatcacggtcggactgatatagcactggcccgtcagggtgcagcccgaacgcagccagcgtatgcaattgacgctctgaggcgcctggctcggcacgcggcgggtcgatgtcttcgattcgcacatgccattcgccaccggcatggcgcgcgagcagccagctgcccaacgcagccaggagcgagccgaagtgcagggggccggtgggcgagggcgcgaagcggccgcggtaagggagtgaggacatgggcagctgaatcgtcggtcaggtgcttgcttgatttcaacccgccacgtccgcagatccgcagcagttcatcctctttgcgtgcggagccactgcctctatgttcaaccgtgtcgtcctgtttctcttgaccaactttgcggtgctgatcctggccggcattgtgatgtcggtgctgggcgtcaatccggcacagatgagtgggctgctggtgatggccgccatcttcggcttcggcggctcgttcatttcgttgctgctgtccaagttcatggccaagcgcagcaccggtgcgcaggtgatcaccgagccgcggacgcagaccgagcgctggttggtggacacggtgcgccgccaggcccaggcggccggtatcggcatgccggaagtggccatctacgacggaccggaaatcaacgccttcgccaccggtgccaaccgcaacaatgcgctggtggcggtttccacgggcttgctgcagcacatgcgcgaagacgaggccgaggccgtgctgggccacgagattgcccacattgccaatggcgacatggtcaccatggccttgctgcagggcgtgctgaacaccttcgtgatcgtgctggcgcgcgtggtgggcggcatcatcgacagcgcgctgtctggcaatcgcgacagcgggcgtggatttgcctactacatcattgtgttcgtgctggagatggtgttcggcctgttcgccacgatgatcgcgatgtggttctcgcgccgccgcgagttccgtgccgatgccggtggcgcgcagctggccgggcgcaacaagatgatcgccgcattggagcgcctatcgctcaatcacggccagaacaccttgccctcgcaggtgcaggcgttcggcatttccggcggtgtgggtgagggcttgcgtcgcctgttcctgagccacccgccgctgaccgagcgcattgccgccttgcgtgcgtccaacggtacggcgatgtaagccagcggtttgcctttcaacaaaaaacccgcgtcatgcgggttttttgttgtggcgtgtttccttgattctcggcagcgcaccgtggcgataaaccgcacacggcgccagccagcagcatggcgccttacgcgggtggctatggcctcagccgaactcgtagttcatcagcgggccggtgggcgcgacgaagtcgccttgcacgtaatcgacccccgcagtgaagaagctgctcatcgactgtgcatcggccacgaactcggccaccgtgaggatgccggtcggctgcgcccgtgaggtgatctcgcggatcttttcctggctttcgcgggcagaggcgatgtcgccggtgatgctgcggtcgagcttgaggaacgcgggctggaagtgtgcgagcagctggaacgaatccagtcccgaaccgaattgctccagccccaccttgcagcccattgccgagaccgaagccaggaattgctgggcgttgcgcaggtgggtgaataccttcgattccggggtctgcagccacaaccgctctcctggcacgccgtagaccgccagctgttcgcggatagtgtcgatcatctgtgggtcggagaacgaattgggcccgatgcgcaccagcaggtgggtcttgtgcccggcgcgctggcgctcgcccagctggcggatggcacgtgccaccacccagcggtcgatctcggtgatcaggtcgtgttcttcggcaatggccatgaacgcattcggcgacatcatctcgccattgcgctccagccgcaggaacgcctgatacagctccagcggctcgccctgcaggttgagcacgggctggtaatgcagcaggaagccgtcgccgatcagcgcctcgcgcaactgttccacccagcgctcgatgcgttcttcctcggcgcgatcggcggcggccgggtcgaagatgctgaccgcattgccaccgagctcggccgtggtgcgcaccgcctcggtgccgcggttgagcacc

SEQ ID NO: 29 - DNA, 3743 bp, Xanthomonas campestris
  CDS (1001)..(2743)
gcgccggaaaccagcctgtatttccgcccgttcatgatcgccgacgaggcgttcctgggcgtgcgcgcggcgcacaaggcctcgtactacgtcatcgccagcccggccggcccgtatttcgccaagggcgtggcgccggtgtcgatctggttgtccaccgagtacgcacgtgcagccaagggcggcaccggcgcggccaagtgcggcggcaactacgccgcctcgctgctgccgcagcagaaagcctacgcgcagggctgctcgcaggtgctgttcctggacccggtggaaggcaaatacattgaagaactgggcggcatgaacgtgttcctggtctacaaggacggcacgctggtgaccccagagctgtccggcagcatcctggaaggcatcacccgcgacagcatcctgcagctggcccgtgaccgcggcatgcaggtggtggagcgcaaggtgtccatcgacgagtggaaggacggcgtggcctccggcgagatcagcgaggtgttcgcctgcggcacggcggctgtggttaccccgatcggcgagctgaagggcgatggctttgcggtcggcgacctgtctgcaccggcgggcgaggtgacgatgtcgctgcgccaggagctgaccgacatccagtacgggcgcgtgccggaccgccacggttggctggtgcgtctgtcctaagccaacacgtcaagccaaatcagggcccctgcgcgtcagcgcaggggcttttttttgcgcgcagtgcagcaagtgcgtgtcatttggtgtcgccgcatgtcatcgcgagactgaacggtgccgtacggtgtcacaaatctgatatttaattttttgaaagtgcgaagttcttattgtccagttcggttggattgcggtaggttcgctgaacggatcgataacaggggcgatccggggacctcggcgctgacctacggaaggcggcgccggataacaccgctccggcggttctcttgatatggaagggaattcgatgtcgactgcgtctctccgcaagcgtactggctcgctcaccatcctgggcgcgtccgccctgacctcgctgctgctcgcgatgccggcctttgccggcgaggtctacctggatggcctggccaccgcacagacccatcagaaattcatcgtgacctacaaggacggcagcaccgcgctggccagcccgtccgcgttgaccacctcgctgcgcactgctgcgcgcgcggtgccggccaaagccggcaaggcgctgggcctgaactcggtgcgccgcctggcgttggggccggaactggtaagggcagaccgcgccctggaccgcgccgaggccgaaaccctgatgcggcaattggccgctgatcccaacgtgcagagcgttgaagtcgaccagatcctgcatgccacgctcacccccaacgacacccggttgtccgagcagtgggcgttcggcaccaccaacgccggcctgaacatccgcccggcctgggacaaggccaccggcagcggcacggtcgtggcggtgattgataccggcatcaccagtcatgccgacctcaacgccaacatcctcgcgggctacgacttcatcagcgatgcgaccaccgcacgcgatggcaacggccgtgacagcaacgccgccgacgaaggcgactggtacgccgccaacgaatgcggcgccggcattcccgccgccagctccagctggcacggcacccatgtggccggcacggtcgcggcagtgaccaacaacaccaccggcgtagccggcaccgcctacggcgccaaggtcgtaccggtgcgcgtgctcggcaagtgcggtgggtcgctgtcggatatcgccgacgccatcgtctgggcctccggcggcaccgtcagcggcatcccggccaatgctaacccggccgaggtgatcaacatgtcgctcggcggcggcggtagctgctcgaccaccatgcagaacgccatcaacggtgcggtgtcgcgcggcaccacggtggtggtcgcggccggcaacgatgcgtccaatgtgtccggttcgctgccggccaactgcgcgaacgtgattgcggtggccgccaccacctcggcgggcgcgaaggccagctattccaacttcggcaccggtatcgatgtgtcggcgcccggctcgtcgatcctgtccacgctcaacagcggcaccaccacgccgggtagcgccagctatgcctcctacaacggcacctcgatggcgtcgccgcatgtggccggcgtggtcgcgctggtgcagtcggtcgccccgaccgcgctgacgccagcagcggtggaaaccttgttgaagaacaccgcgcgtgctttaccgggcgcctgctcgggcggctgcggtgccggcatcgtcaacgccgatgccgcggtcactgcggccatcaatggcgggagcggcggcggtggcggtggtggaaacaccttgaccaacggcactccggtgaccggcctgggcgcggcgactggcgcggaattgaactacaccatcaccgtgccggccggcagcggcaccttgacggtgaccaccagcggcggcagcggcgatgccgacctgtatgtgcgcgccggcagtgcaccgaccgactcggcttacacctgccgcccataccgcagcggcaatgccgagacctgcaccatcaccgcaccgtccggaacgtattacgtgcgtctgaaggcctacagcacgttctccggcgtcaccctgcgcgccagctactaaccgcgcgtcacgcgcgccccggcactgcgccagggcgcgctcctcatctgcaatccactccacgcctgccgattcggcaggcgctgggaatgtgttgccgcgtggccgggacggacaccggtcagtcgtttcgacaggcacctgcctgtgcacttccagacctgccatcgcagcgcgcgatggggtccggccatcacgccgcatctgcggcacctcatcactgaaaggaacgatcgatgtccaatgtgtcgcaacgtcgtgcgcctacgcgcgcgctggtagtcctgggtgtgtccgcgctgacctcgctgctggcggtgccggccttcgccgggcaggtgaatctgtccgggctggattcgcagccgacgctggatcgcttcatcgtcaaataccgcgacggcagcagtgcgtcggtgagcccgacctcgatgcgcgcctcgctcaattcggtggccactgcggtgccggcacgcgccggccgcgcgctgggcctgaaccacgtgcgtcgcaccgcgctgggctccgaagtgctcaagaccgaccgcagtctggaccgcgccgaagccgagacgctgatgcgccgcacatcgccgccgacccgagcgtggaatacgtggaaatcgatcagatcatgtacccgacgctgaccccgaacgacacgcgtctgtccgagcagtggggcttcggcaccaccgcatcgggcatcaacgtgcgcccggcctgggacaccgccaccggcaccggcgtggtggtcgcggtgatcgacaccggcatcaccagccatccggacctcaacgccaacgtgctgcccggctacgacttcatcagcgatgcggcgcgtgcgcgcgacaacaacggccgtgacagcaacgcggccgaccagggcgactggcgcacggccaaccagtgcggtaccggcgtggcagcggccaattccagctggcatggcacgcacgtggccggcacgattgccgcggtcaccaacaacagcaccggc

SEQ ID NO: 30 - DNA, 3452 bp, Xanthomonas campestris
  CDS (1001)..(2452)
cgcgcggcaccacggtggtggtggcggcaggtaacagcaatgccaacgtggccaatttcaccccggccagctgcgccaatgtgatctcggtggcctcgatcacctcggccggcgcgcgttcgagcttctccaacttcggcaccaccatcgatatctccggtccgggctcggcgatcctgtccacgctcaacagcggcaccaccaccccgggcagcgccagctacgcgtcctacaatggcacctcgatggcggcgccgcacgtggccggcgtggtcgcgctggtgcagtcggcagcgtcgcgtccgttgaccccggcggcggtggaaacgctgttgaagaacaccgcccggccgttgccgggcgcctgctcgggcggttgcggtgccggcatcgtcaacgcagccggcgcggtcagccagacgccgtaacgcgacgccgtaacgcgtatttccccagcgcacagtgccggcgtctgtcagagcgctcccgacgcggctgtcggtgcctgtctctctctcttgtgccccgcccaggcggggcttttttgtgcgcgcgctgcatgggccgggacgtgcgccaggggtagctgaacgaacgcaagtgggcggatgctgcctggcttcagcgccattcgggttcgccgatagcctcgtcacgtgggggctgcgtcatgcaatgcccgcgcatccgacgcggcgttgcagtccctgcgctgcgcgtcatccgagtaccgatatcctggggagggcgattcatgaggcagtcaacggttggcgcagtgcgctacgcggcgtgtgtgtgtgcgatggcgtgtgtgtttcaggtatcggccggcgaggtcaacctgcagggcctggcatcgggctcgagccaccagcgtttcatcgtcagctacaaggaccgcgtaggcagcacccgcgcgaccgggctgtcggctccgtggggcgagatcgcccgcgccgtccctaggcggcaagggcgcgcgctgggcttgtcggccatgcgtcggctcagtgccggcccgatgctgctggtggccgatcggcgcctggaccgggtcgactcggaaagcctgatgcgccggcttgccgccgacccgaccgtcaagcgggtggaagtggacgtgctgatgcggccgctgctggcgcccaacgaccccggcctgccacagcagtgggcgatgggaaccaccaccgccagcctcaatgtccgcccggcctgggaccgcaccaccggcaagggcatcgtggtggcggtgatcgataccggcatcaccgcgcatccggacctggccgccaacgtgttgcccggctacgacttcatcaccgaccccaccgtcgcaggcgacggcaacgggcgtgacaacaatgccgccgatcagggcgattggtccgcggccaacgcttgcggtgcgggcgcgtcggcgtccaattccagctggcatggcacccatgtcgccggcatcgttgccgcagttggcaacaacgcggccggggtggtcggcactgccttcaacgccaaactgttgccgttgcgcgtgctcggcaaatgcggtggctacatgtccgatatcgccgatgccatcgtgtgggcatctggcggcaaggtgaccggcgtgccggccaatccgaatcccgccacggtgatcaacttgtcgctcggcggctatggcagctgctcgacgatcatcggcaatgccatcaccggcgcggtcacgcgcggcaccgccgtggtagtggcggccggcaacagcaatatggatgtggccaccagcatgccggccaattgcgcgaacgtgattgcggtggcggcaaccacctcggccggcgccaaggccagcttctccaacttcggcaagggcgtggacatcgccgcgcccggccaggcgatcatttccacgctcaatagcggcaccacggttccagccaatccggcctacgcggtgtatagcggcacctcgatggcggcgccgcatgtggccggcgtggtggcgttgatgcagtcggtggcacttaacccgctgacgccggcaacggtggaggcgctgctgaagagcagtgcacgcccgttgccggtggcgtgcgctcccggctgcggtgcgggcctggtcaatgccgatggcgcggtggcggcggtgatcaatgccacgctgctgaccagtaatgcagtacgcactggcctgagcgcggcgatcggcgattcgctgtattaccaggtcaaggtgccggcaggcacgcgctcgctcaaggtcacgctcagcggcggcagcggcaatgccgacctgtcactgcgcgccaatgcactgcccaccgacgctgcctttggctgccgcagcatgctggtcggcaacaccgaagcctgcacactgaccgcacctgcggcaggcacgtattacgtgcgactgaaaggaacgctggcgttctccgcggtcaacctggtggcgacgtactgatagcagggcagccgcctggccgtgcgaggcagtgacgcgctggtgcctgaggcaaggcgccagcgcgtttctatgtatggcggacgcaaggctggcttacctgcgctatttcgggcgtggcaggctcactcgcagagcgggtaagcgcgcgcgccgaggacatgcacaatctcggcgccggccagttccagcagcaggccacgctggccggcgttgatccacaacgcaggtgcgtcgagcacgctggattcgatcagcaccggcgtgggccgctgctgcccgagcgggctgatgccgcccaccttgtagccggtgcggcgttcggcatcggccacctccatcatgcgcgcggacttgccgccgcaggcgctggcaagggtcttcaacgacaagcggcggtcgcacgggatcaccacgcagatcgcgtgctcgtccacccaggtcatcagggtcttgagcacctggtgcggcggcagttgcagcgcctgcgcagcttgcaggcccttggcgtcggcatcggcctggtactgataaggatgcagcacataggccacgccggccgcatccagtgcacgggttgcgcgggtggccttgctcatcgcttagaccggttcgaaccggttggccgccacgttcttgcgcaccgtttccgggttccagatccttccgttcatggcgatgtagacgccggccggcagcgtctggacggccccgaccgcgcagccgatgttgaactcggcatcggagccgcgaaagcgcgccggattgagtgcgccggtcatcacgatggtcttgtcggcgatcgactgcagcaccttgccggtttccaccatcgaatcggtgccgtgggtgatcagcacctgtcgggcgggctgcgcctggatggtggcgcggatcagctcgcggtcttcctgggtgatgtgcaacgaatccttgcggatgatcgggatgaccgtgaagcggaaggtcacgcccagttccttgaggatctggccgatct

SEQ ID NO: 31 - DNA, 3650 bp, Xanthomonas campestris
  CDS (1001)..(2650)
cgaggacctgcgcgccggccgcctggtcgaagtgatgcccgactacccgccgcccacctcgcagatcagcgcggtcatgccggcgcggcaattgcagccgccccgcgtgcgtgcgtttgtggactttctgctcgaacacttcggcgagacgccaccctgggaactgcctagtgctgcaagaggtggtgagtgacgccaccgcagcgtgccatcagtatcctgggttcagagcacgcttcagcctacgtcggccgctgaataacttcgcgttgcgattgattgtttggatcgatcacgtcaaaaaaatcccaatcgcaagcacatgctgtgccgttatccgcagcagttgcgcatagaactacatgtcactgcaaaaatcgctgctgcatcacagccggcgccgcgttctgcgatttgtcgtgctgcatcgctgcagttacatctgtgcccagtcgcttgcagatcacgcgaccgcgccgagccgagcattcccaatgctcgccggctgcacaggatcgcctgcgtcgttgcatgacccgggcgcatggatagataccgaccggactcgatgaagatggatatgccttcgctcctgatgtacccgtacgtgccacgccggccccgcgatattgcgtggtccgctgccgccgttccactgcgatatcgcacacaacatctgcgctgatagcccgcattaaccgcccgagtactccccgctcggcgcacgacgcacgacgcccttgatgcatcacacacgaccatagcggccgtgtacgcacgacgcatgcgcgccattgcgagcgcctgcatgcgctacgccgtcgattacggcaaatctcccgctcgcacccaaatgggactctgttgatgacttcttttccgcaacggcaattgctgttctccgcgctcgtgctggcgctgtgtagcgcggcatcctcggcaccgggtgttggcgcaaaccccggcaacatcacgcgcccaacccaggccaccagaagaaagtgacccgcttttccgctatcaatggcatttgctcaacgatgggcaagaggtcattgccgacactcggccacgcgccggaacggatctgaatgtgggcccgctgcatacgttggggctgcgtggacaaggcgtaaccgtcgccgtcgtggacgacgcactcgaactacgccacccggatcttgttgccaacgtggtgcccggcggttccaagaactttgtcgatggatcgaatgatccaacgccacgcaacggcgcttcccacggcacgatggttggcgggatcatggcggcagtgggctggaacgggcttggtggtcgcggcgttgcgcccgatgcgcgcctgaaaggtttcaacatcctttcctcagaagaaagcccgaccgacttcgataccaatctacgcgcctcctggggcgattcggtgcaatcccgggatgtggatgtattcaataacagctttggctcagatctgacgtactacccgacaatctctccagcagcagagcgctcgcttgaccgcttgatgcgtcgagcaagaaacggcaaggggggtctgtatgtccaggcagcagggaacacgttcgatagttttacggttctcgacgaccaggggaactgggtggagcgatgccctgtgctggcccgcaccttgggagtcacttgctccacgccggctaccgatccgttgagtaaccaacccctgatcatcgcgacgggcgcagtcaatgcacgcggcctgcgctcgtcctactcttcagccggcgcggcgctatgggtcaccggcttcggcggggagttcgggctgcaacgccgctactttggcgaccggccgcggcccgcgttgttcgatcccgccatcgtcaccaccgacctgactggctgcgccgtcggtgacaacagggacatcgccggccagccgccgatcaatgcattggcttcaagcagctcaccgatcgacgcctcttgcaactactccgcggcaatgaacggcacctccgcagcggcacccacggttgctggggtggttgcgctgatcgtgcaggccaatccgtcgctcaccgcgcgcgaccttaagtacatcctggcaacaagcgcgcgccagatcgatcctgcgcagcaggtgatccgctaccaaggcagcgtgatcgagccgggctggatcaccaatgcggccggccatgcatttagcaactggtacggtttcggcctggtggacgctgccgaagcggtctatcgcgcctatgggttccagccgcttccgccccagcgcgatctgggctggaaagcggcacgcgcaggcacatcgacgattggcggaccaaatgccgcggcaaccttgcggttgcggctcggggacacgctcaagatcgacaccgtgcagtggtcgatgcagaccacccacaagacaccgagcaatctccgggtagtgctgatctcgccgagtggcacccgcagctacgtgctcactcccttccaggccttggatacgatcacgcagggggccggctttgagataccgctttcgaccagcaatgccttcctagacgaaaacgttgccggcacctggacgctggaagtcaccgacatgaccggctcggacgttcccgcccaactgacgggcttcaaactgcgcatcctgggacattgacatgaacctcgctctcggtacgaccctcatcctttgcgcgcttgccagcatgccggcctctgcgcaagacaccgaccggctcgcattgcagcgtgccgcaaccgggcaaggcttcaccatcggcgataccaacttcaggttggtacccaacgccagagtgctacgcaatacaacgccgccgccccgcgcagatgtgcctgcaatgcgccgtagcaccactgccgctgccggcgtaacgctgcagcacattggcccgtatgtcatctccagcggcaactcaccgcgctcaccgcgtaccacgcccgtcggcgacgtggaggagccgtcgttgatggtggcaatcaatcagcgcaacggccagccagtggtggcgtcaccacgcctgcaggtgtatgccggcgatgccgatgccatcgttcgcttggcgcggcaaaccaatggtgctgtcttggcagcatcacaggatggcacgggcgtgatcggctatcgttcggtcccggaagcactcgcagcgctaccaaccgtaaggcgcggcggcggcgttgacgacgttgtccttcaggtgatccaggcagtcccacgcctgcgctagtgctagccgccgccaccgtgtggaacgattcacacggtggcggtgatgtaccgggcacgactgacggaggagttaaccggcccgatcatgcgtcgcccgctgcgatctccgtgttcgtccttcaggggaacacgcgtttgccgacgatccagttcacttaagcgtactcgtgcggtacaggcgcttgcgcaacccgcgccgctgcacctcgctgagcaaatgcccatgagtgtgcaacaggccaaggatctgctggtgcgccagttgggtgcgccgtgcgagctcggccgttgccgccagctcgatcgctttccagtcggcaggggttgcgggatctgcggggagtgaacgctttgcggccatgaggggacgggcaccagggatgcgccgcatgatacgggctgcgtg

SEQ ID NO: 32 - DNA, 3044 bp, Xanthomonas campestris
  CDS (1001)..(2044)
ctcggctctggtcgacggcggcgtgctgcagcgttatgtgctgggcagctattcggcgcgcaagctcgggctgcagaccaccgccaatgccggcggggtgcacaacctgcaggtgaccgccaatgccggcgacctggcatcgatggtcgccggcatgtcgcgcgggctgctggtcaccgagctgatgggcaacggcgtcaacgcggtgaccggcgactattcgcgcggtgccggcgggttctgggtcgagaacggcgagatcgcctatccggtcgacgggctgaccattgccggcaacctgcgcgacatgtttgccgggatcgaagcggtgggcagcgatgtcgacccgcgctcgcacgtcaagatcggctcggtgctgatcaatcgcatgaccgtggccggcgacagctgagtcacccgcccagtaactgcgcccgccagcgtcccggcgctggcggacccatggcttcccgcactggtggcgcgcggattggttgctatagtccgccgcacaccacaaccatgcatggggagacatgatgagcgagttcgacacacacgccgcgccaccaccgccgccgatcagcaccggcacgccctcggaagagcgcacgttggcactggccgcgcacctgctgggcatcctgacgtccttcatcggcgcgctggtgatctggctgatcagcaaggacgccggcccgtccaagccgtttgccaccgaccaggccaaggaagcgctgaacttccagatcaccgtgatcatcgcctacgtggctgcggtgatcctgaccatcgtgtcgttcggcatcctgttcttcgtgccgacgctggtgtgggtcgccaacctggtgctctgcatcctggcggcggtcaaggccaacagcggcgagagctaccgctatccgttcacgctgcgcctgatcaagtaattcttgattacgcgtaaaacaaaggcccggcagtcatgccgggcctctggcgtttttagaaagtcacgcaatcagttctgcgccggggtgttctcggcaaagtactcgtggctgtcggcattggtgatcgcctgggccgggttgctgatcgccaggctgcgtgcgccggactgaccatacacgcggtcctgggtgccggccaccacggtgaagtggctggtctcgtgcaccagcgtgccggccttggagtcggtgccggttgtcgacgcgctccagaacgcgttgcagacatggatctcgtacggctggttcggatagacgtaagcgaaggcgtccgccagatccgcttcgcagctgcagttgatcgtcagctggccattgttctggtccagcgcgttgtcgatgttgacgaagttggagctgacccggctgtagcgcgcggcgttgtaggtgccgaaccaggtggtgtagcgcgccccggtgctgccgccgttgaggtaggtacgcgcgttctgcgagtaattacgcgcggcagcgacgccactggcggcctggttggtgcgggtggtgctgcagttgagatagttgatgccgttgaccacggcggtcgggccgattgcgagctggcgctatacaccacgtcgcgcgccatccagccaaacagtgagcggggtgctggtggccactgccggcaggccgtttgcggccttcatcgggctgccatcggacagcgaggcgtactgcagcgacgagcgcacctggatggtgtagttgccgctggtggacaggtcataggcgccggccagatccacctcgccctggacgctctggcccggctgcaggatggtgaagtcggctgccttgggcaggccgcgcttgaccaggcgcccggtgtagtccaccggcttgccgtcgcggctcacctccaggatgccgttgtccagcgacttcagcggcagctgataggtcggaacgcgggcaacctggctaccgttgttggtgacggtgaccgcgatcttgccttgatgtcgcccggcctggtcggccactggcgccagttcgatggtcaacggaaccggtccgcgcacggactgtgcctgcgccgaaccgagcacgccgacgacggccagcgtgcctgctgcaaacgatgcaagaaaaacattcttcaccagtgactctccttggtcgggggatgcccgcaagcgcgggcaggccaaacctaggcagagcgagcgaacgcgacaagccgttcaattagcgatatttctggatgtggcgcatgggttatgcatccggatacatcactggtgtagataattcagcgataaagcctgctggcggtcacggaattgtggtgcctgtgtgtcgccggctgacacaaacgctggcgaccaatgtgccgttttgtgaccaggcgcgcctgttgcctgaatggacccgcgtgatcgaccgtgcggtggcgcacggaatgcgcaaacgtgtcgcgtaacgcgaacaccgggaaggtgatgcccgcgccagcgctgtgattgcgttggggtctagcaggggcggtgatgtgcggagcgatgcgtcggatgtcgccgcgccagacacccacgcaccatcacacgcgcttggcagccgcgcatgaggcctggttagccgtgcctagtgggcgcggtcgaccgcgaagcggccgaggctggccaaggtggcgccggattcgccgtagggcagcaacagcgcatgcatatggctggccagttccgccagcttggttttggcgccgtccatgcccagcaaggcggggtaggtggacttggactgcgccgcatccttgccggcagtcttgccgagctgggcggagcttgattccacatccaggatgtcgtcacgcacctggaacgcgagccccagcgcgtcggcaaacgcatccaggcgctgctggtcggcaatacccgcgccgcctgtcagcacgcccatgcgcaccgcggcgcggatcagcgcaccggtcttgagtgcatgcatgcgctgcagtgcctccagcgcctgcacctgcccggtggcgtcgatatccagcgcctgcccgccacacatgccggccgcaccggcggcagatgccaggctctgcaa

SEQ ID NO: 33 - DNA, 3788 bp, Xanthomonas campestris
  CDS (1001)..(2788)
cacaagtagtttcgggggttcgccttaggcggctgtagaactacaactaaagccaattaagcggcgatagcgaagtccgaaccgtagttgtcgtcgttggcaactaaaagtttgctgctggattaacgaggaaagctgccccctcggcatgcgccaagcgacttcgcgacccccgtcgaaaccagtgcacccccggggaaagcatcaaaacgctgacgtgaccagtgtagggatcggcgcgcccggtcacaagtagtgagataggcagcgcttacagtggcttcagttgcacagtccggaactatccggaactgaaggagctactggatgagcacaggcaagcgccccaccggcccgcgcacgacgcgcgccgcaaccaagaaggccgccaagaccgccccgccggccaagcgccgttcggggcgtcaggccgccgccaaggcggaaacgcaagaggaaaacggccatcgcacgcgggtgcagggcaaggcccgcaccgtcatctacatccatggcatcggcaacaagccaccggccgaggtgttgcgctgccagtgggacaaggccttgttcggccgcccgatgggtgagcgcacgcggctggcgtactgggtcaaccgcgagcgctacccggtgccggagcccggcagttgcgatggccgcgatgtggggccggcgttgaaccagagcgtgcagcgggcgttgagcacgctggggctggtgccgggcgagcaggacctgcatgcgctggccgatgcgctggcacagagcgagcaggagcgtgctgacctgcaccagatgctggacgagctgcaggccgcgccactcgccggtggcatgcaggcgaaggggccgatcgatgcgatcaaccgggtgctgctgcggctgatttccgcggcgctgctgcaggacgtgcacgatctgttctttgtcccggagcgggccgcgcagatgcgcgaaagcctggcgcaacggttgcgcgccggtggcggtcccgttcgtggtggtggcgcacagccagggctcgatgattgcctacgatgtgctgcggcagttgcaggccgatggctgcgaggtggcgctgtttctgaccctgggctcgccgctgggcttgccgcaggtgcgcagcatgttcaagcgctggaccgggacgcgcaaactgccgttccccgactgcgtgcggcgctgggtcaacgtggccgaaacgcgcgatccgatcgcgctggatccagacctgaccgacgacatagccaatgccaaagggcggttcgaaaacatcgccgccgcgcggctcaacccggactgggagcacaacccgcattcgggctcgggatatttggccattgcgcaggtgcgtgcagcggtgcggcaggcggtgggtgtgggcttcgatcagccggtctccaatgcggtgctgatcaaggacctcagtgagcagctcgaagcgcacggcgcggactatcggcacgaggtgttgatcgaactggaccgtcgcgtcctgggtgcggacccggccggcgtgcgtgcgcagttgatcacccacatgcgccagattgccgggcatagcaccggcctggacggcgatgcattggacgaggcgatcgaactcgaagacagcctgcagcgcctcatttcggcgcggctgacgcggttcgagatcgaaacgctgcaaagccgctaccgcgcactgggtttccggcgcgtctggcgcgatgccggcaagcgggcgctgatccacgtctcgggcaacgtgctgcatgtggatgcagcacgcacggcgtatcgcgcacgtgggcagcagatcggctgggcggtcctggataccggcattgcggccgcgcatccgcatttcttcgtgccgggcgagcgcgacaatgtggtggcgcaatgggattgcacgcggcgcggcgcgcccaagcggctgacgcgggccgatggcaaacggttcaccgcactcgacgggcacggccatggcacgcacatcgccggcatcatcgccggctgttgccgggcggcggtgcctgatgccagtggcaagccgggcgaactgctggaatttgccggcatggcgccggagacgcaactctatggcttcaaggtgctggacgacgccggcaacgggcgcgattcgtggatgatcaaggcagtgcagcacgtggccgatctcaacgagcgtgccggcgagctggtgatccacggcgtcaatctcagcctgggcgggtacttcgatccggaaagctacggctgtggcttcacgccgttatgcaacgagttgcggcggttatggcggcagggcgtactggtagtggtggcggccggcaacgaaggcctggcctggctgatgcagaacgacggcggcacctatccggcgaacatggatctgtcgatcagcgatccgggcaatctggaggacgcgatcgtggtgggatcggtgcacaagagcagcccgcacaattacggcgtgtcgtatttttcatcccgcgggccgaccgccgatggccgctccaaacctgatgtggtcgccccgggcgaaaagatcctgtccgcttactacggcttcgacccgcgcgacccgtccagcctgatggtcgagatgagcggcaccagcatggccgcaccgcatgtgtccggcgtgctggccgggtttttatccgcacgccgcgagttcatcggctttccggaccgggtcaagcaactgctgctggacacctgcaccgacctgcagcgcgatcgttacgtgcagggcaggggggtgccgaatttgatgcggatgcttggagagacgtgattcgggagtcgggatttgggattcgcaaacgcggtggttggctgaacaaaaacggccgcgatgcaggttgcagcccgaatgctggctaagagcggtcgcgtcacatggcgtggcgggatcgcacggcctcaaagcagcccaaaccatcgcaacttgcggtctcagcaaatgctcagctcaagcagccccgttcttgcgaatcccgaatcccaaatcccggcgactcacgcatcccgattatgtcgccgcatcacgcgctgcttgtcgcgctgccagtcgcgttccttggcggcatcgcgcttgtcgtgcgactgcttgcccttggccaatgcgatctccagcttgaccttgttgctgctccagtacagcgcggtgggcaccagcgtgtagccttcgcgctccacgcgggtcagcaccttgtcgatttcggcccggtgcagcagcaacttgcggtcgcggcgctccaccgggatcacgtgggtggaagcctggatcaacggggtgatctgcgcgccgatcaggtaaatctcgcccgaccgcacgtaggcgtagccgtcgacgatgttcgcgcgcccggcgcggatggccttgacctcccagccctgcagggccaggccggcttcatagcgctcctcgaggtggtactcgtggcgcgcacgtttgttcagggcgatggttttcgtggccgtcgcgctctttgctttatccttggcaggtttcttgctcatgccgctattgtcgccgattcggcgtcgccaaacgatcgcccgtctttcacaagtcagagaatgcctaccatccgtcgcagcgccctggtcgaacacagcgccacccgcatgttcgatctggtcaatgatgtcgccgcctatccgcgccgcttcggctggtgcgacgctgcgcatgtgcttgaagaggacgacgcccacatcgtggcgcggctggacctggggctcggttcgttccgcacctggttcaccaccgaaaacatcctcgagcggcc

SEQ ID NO: 34 - DNA, 3884 bp, Xanthomonas campestris
  CDS (1001)..(2884)
ctgcatcagcgacggtcgcgagcagcgatgtcactgacagcagcgcaagcgtgcagggcacgccgaccgctgcggtcgttggcacggccacgccggcagccacctcgacggcagttggcaacgccgctgtggccaaccacatcaccggcaccgcgattggtggcagcgcgtatgcgcacggccccaacgacaccgcgattggcagcaatgcacgcgtcaatgccgatggcagcaccgcagtgggtgccaacacgcagattgctgcggtggcgaccaatgcagtggcgatgggcgaaggcgcgcaggtgagtgcggcctcgggcacggcgatcgggcagggtgcccgcgccagtgcgcagggtgcggtggcactcggccagggctcggttgccgaccgtgccaacacggtctcggtgggcagcgtgggcggtgagcgtcaggtggccaatgtggctgccggtacgcgcgccaccgatgcggtcaacaagggccagttggatagcggcgtcgccgcagccaacagctacaccgacagccgttacagcgcgatggccgacagcttcgaaacctatcagggcgatatcgaagaccgcctgcgccggcagaaccgtcgcctcgaccgtcagggcgcaatgggctcggcgatgttgaacatgtccgccagcgtggccggtattgcctcgcaaaaccgcctcggtgccggtgtcggtttccagaacggcgagtcggcgttgtcggtgggttatcagcgggcaatcagcccgcgcgccacggtgaccatcggtggcgcattgagcggcgacgacagctcgatcggcgtaggtgccggtttcggttggtaacgcacggttgaaaacagcgacggcgccgtcgtgcgccgtcgcgacagagcaaggcctgagggggcgggcgaggaccgtggcttggccggccaacggattcgcagggagtgatggccgtactgacgcctgatccacgaaaaactgaagaggacttcaccgtgatcgacaagaatttccgtctcaacccgctgactggcgccatcttgatgatggccctgggtgcttcgggcaccctcgttgctgcccccgcgttgcaggtcaaggaaccgacccaagccgcgccggccgccagcgatttcagcagccggttgatcgtgcgttacaaggacggcacggccgccgcgagcgatcgcagcagcaagctgggcgtggtgcaatcggccgtgggccgtgccgcaccggtgaccggtgcgcgtgctggcgccaccgccgccaaggcgacctacctgcgcaagctgggcattggctcggacctgatcaaactgtccggcacgctgaccgccgcacaggtggacaaggtggtggtcgagctgaagaacgacccgtccgtggccgaggtgcagatcgaccgcatgctgcgtccggtcgagatcaagaagagcgttgcggcgaccgacgtgagcccgcagctggtccccaacgacccgctgtatgcgcagtaccagtggcacctgagcaaccccaacggcggcatcaacgcgccggccgcatgggacctctcgcaaggtgccggcgtggtggtggcggtgctggataccggcatcctgccgggccatccggacttcgccggcaacctgttgcagggctatgacttcatcaccgacgccgaagtctcgcgtcgccccaccgacgcccgcgtcccgggcgcgctggactacggtgactgggaagaggccgataacgtctgctacgacgggtcggtcgcccaggaaagctcctggcacggcacccatgtctcgggcaccgtggccgaggcaaccaataacggtttgggcatggcgggcgtggcgccgaaggccaccatcctgccggtgcgcgtgctcggccgctgcggcggctacacctccgacatcgccgatgcgatcgtgtgggcctccggcggtacggtcgatggcgtccccgccaacaccaacccggccgaagtgatcaacatgagcctgggcggcggcgagccctgcgatccggccacccaggtcgcgatcaacggcgcggtgtcgcgcggcaccaccgtggtggtggcggccggcaacagcggcgaagatgcggccaaccattcaccggcgagctgcaacaacaccatcaccgtcggcgccacccgcatcaccggcggcatcacctactactccaactacggcagcaaggtggacctgtccggtcccggcggcggcggcagcgtggacggcaacccgggtggttacatctggcaggccggctacgacggcgccaccaccccaacctccggtagctatagctacatgggtatgggcggcacctcgatggcgtccccgcacgtggccggcgtcgtggccctggtgcagagcgcgtcgatcggcctgggcgatggcccgctgaccccggcggcgatggaagccctgctcaagcaggccagccgccgcttcccggtgaccccgccgaccagcaccccgatcggcagcggcatcgtcgacgccaaggccgcgctggaagcggtattggtggagccgtgcgacccggacaccgagacctgcgcgccggccgccattgccctgaccaacaaggcgccgctgaccggcctgagcggtgagtacaacagctccacgttgtacagcttcgaagccaaggccggtgcggtgctgagcttcatgacctacggcggcaccggtaacgtgtcggtctacgtgagcttcgaagccgagcccaccgcaaccgattacgacgccaagtccacccgtccgggcaacagcgaaacggtgcgcttcaccgcacccaaggctggtacctactacatcaagctggtgggtgcaggggactacgcgaagctgacgctcgtcgcgcgacagtaatcgctgcaatagtgtgaccgaatgcccccggccaatcaccgggggcattcgtttttcagggcctttcgcagtggtgcggttgatcgtcgcggcaaactgctacagtcggcgccggacttgtggagacgagtgtgaacgcggttgccgtcgatccaattgagcagcgaagtgcagaaacgcgcatcgtcgaagcactgcttgaacgccgtcgtctgaaggacaccgacctgctgcgcgcgcgccagctgcaggccgaatccggcatgggcctgttggcgctgctgggccggttggggctggtgtccgagcgcgatcatgccgagacctgcgccgaggtgctcggcttgccgctggtggatgcacgccagctcggcgacaccccgccggaaatgctgcctgaggtgcagggcctgtcgctgcgctttctcaagcaatttcatctgtgcccggtcggcgaacgcgatggccggctcgacctgtggattgccgatccctacgacgattacgcgatcgatgcggtgcgcctggccaccggcctgccgctgctgctgcacgtgggcctgcgctcggaaatcgacgacctgatcgagcgctggtacgggcagggccgcagcgcaatgggcacgatcgtggaaaccgccgacggcgatgccagcagcaccgacgacatcgaagcgctacgcgatcttgcctccgaagcgccggtgatccgcctggtgaatctggtgatccagcacgcggtggaattgcgcgcctcggacatccatatcgaaccgttcgaaagccggctcaaggtgcgttaccgcgtggatggcgtgctggtggaaggcgaaagcccgccggccaagctcaccgccgcggtgatcagccgcatcaagatcatggccaagctcaatatcgccgagcgccgcctgccgcaggacggccgcatcatgctgcgcgtgcagggcaaggagctggatctgcgtgtcagcaccgtgcccaccgcgcatggcgagag

SEQ ID NO: 35 - DNA, 4880 bp, Xanthomonas campestris
  CDS (1001)..(3880)
gccgcaggccaccacgccatcgggcatggcttccagcaacaggctcaacgtctgccgctgcgcctgcgcatccaggcggtcgcggcgcgcctccagctgattgacggcctgacgcgccagccggatcaaggcctgtttctggttctcatcgagcacgcgtggcctggtgctgagcatgcacaaggtgccgtaggcatagccctcgcggccgatcagcggcacgcccacataggagcggatgcccggcgcggcggtcaccaacgggttgtgcgtgaaccgtgggtcggcgagggcatcgggcacttccagcaggtcggtgcccatcaccgtgtacgagcagaacgacacgctgcgcggcgtgccttccagatcggtgccgcagcgcgccttgaaccactggcggtcggcatcgagcagggaaatcagcgccatcggcgaaccgctgatatgcgcggccagccaggcgaggtcgtcgaattcggcttcggccgcggtgtccagcacccccagccggcgcaccgcatccacgcgcaaggcgtcgttggcggggatcgggacgggcggcgacagggcggtcatgaaggaatcggctgacaggtcggatgacggtgggtcgccccaggtaacggcaaggcgacgggcaacttgagccggcgcggccggggccgcttggagcagctatcaaaacgactgcgcagccgccaggcgggcgcggccggtgctcggaatcggcatgtacccccgtacactccggttcctccgcgccgtccgcacccacctgacggctgctcgctacgttttgttagccgcgcttagtcccgtggccattgccgctgtgcccacgtggccggcatggtcccgatggcacgcccatctgctcgctgcggtgccgaaggtttgccaggcatccggccagcccgccctcccctgaccaccggcatttgcgacgccgccaagcagccactaccctcggccgattgtcctgcccctggagtgtcacatgctgcgtccgttgtccttgttgatcgccggcgtgctcggcgttgctgccggtaccgttgcgcccgccgcacaggccgccccggccagcctgtccaagactgcgaacagcagcgccattcccgacatcgcctacacccgtttcaccctgcccaacggcctgaccgtggtggtgcacgaagaccacaaggcgccggtggtggcggtgagcatctggtaccacatcggttccggcgacgagccggccggcaagaccggcttcgcgcatctgttcgagcacctcatgttctccggctcggaaaacaacaagggcagcttcttcgcgccgctggaaaaggtcggcaccaccgacatgaacggcaccacctggttcgaccgcaccaattacttcgaaaccgtgccgaccaccgcactggataccgcgctgtggctggaatcggaccgcatgggccacctgctcggcgccatcggccaggaagaactcgacacccagcgcggcgtggtgcagaacgaaaagcgccagggcgaaaaccgcccctatggccgcgtggaccagaacatcctgtccaacctgtttccggccaaccatccttaccagcacgacaccatcggctcgatggaagacctggacgcggcctcgctggcggatgtcaaacagtggttcaacgataactacggcgcggccaataccaccctggtgctggccggcgacatcaccgtggcgcaggcccgcgccaaggccgagcagtactttggcgatatcccgtccggcaagccggtggcgcgtcagcagccgtgggtgaccccgctggccgcacagaagcgcggcgtgcagcacgaccatgtgtcgcagccgcgcatctaccgcacctgggccgcgccgcagttgggcaccgacgacatgatccagctggatctggccaccaccgtgctgggcggcggcaagacctcgcggctgtaccagcgcctggtctatcaggaccagctggtggacgatgtttccgcgtcgatccagccgttcgcgctggccagccagatgcagatccaggccgacgtgaaagatggtgtggacccggccaaggtcgaggccatcatcgatgaagagctgaagaagttcctcgccgaaggccccactgccgacgaactgcaacgcgcccaggtggcctaccgcgccggcttcgtgcgcgggctggagaaggtgggcggtttcggtggcaaggcggtgatcctggccgagggccaggtctatcgtggcgacccgggtgcctacaagcaggacctgcagcgcggccaggccgccaccatcggcagcgtcaagcaggccgcatcgacctggttcggcaagggcgactacctgctcaccgtactgccggccggcaaggacttcgaccccgccgccgaagacaaggcggtggtcgcgcgcggcaccgagccgggcaagcccgcgcccaagttgccggcggcggccaagtacaaagtcaccgccagcaagctcgatcgcagcaagggcgtgcccgacaccgccagcttcccggacctgagcttcccgcagctgcagcgcggcaagctgaagaacggcgtggaagtgatcctggccgaacgtcacaccatcccggtcacccaggtggagctgttgttcgacgccggctacgccgccgaccagggcaagaagcttggcaccgccagcttcagcgccgcgctgatgaacgagagcaccgcgtcgctggattcggtggaagtggcgcagcgccgccagcgcctgggcgccatcaccgaggtgggctgcgatctggacagctgcagcgcctcgctgaatgccctcaacgaccagctgcagccgtcgctgcaattgttctccgacatcgtgcgcaacccggccttcaaggcggcggacatcgaacgcatccgtggccagtggctggccgggatcgcgcaggaaaagacccagcccaacagtctgggcctgcgggcgctgccgccgctcatgtacggcgacaagcatccgtacggcatcccgcttaccggcagcggcaccgaggcggcaatcaagagcctcaatgccaaggacctgcaggccttccacaaccagtggctgcgtccggacaacctgcgcatcctggtggccggcgataccacgctggcgcagatcatcccgcagctggatgccgccttcggcgactggacggccccggccgcaccgttgccgaagaagaacctggtcaacgtggccgcgcagcccaagccgcgcgtttacctgatcaaccgcccggacgccccgcaatcggtcattctggccggcctgttggcaccgtcgaccaaggcgccggacaacctggcaatcacggtggccaatggcgctttcggcggcaccttcacctcgcgcttgaacatgaacctgcgtgagaacaagcgctgggcctacggtgcgcggacccaactggtggatgcgcagggccagcggccgtacatgtttgccgcgccggtgcagaccgacaagaccgccgagtccgccaatgaaatcttcaaggaagccacggccatcatcggcgacaagccgctgaccaccgaggagatcgagaagatcaagaaccagcgcatccgcgccctgcccggcagcttcgaaaccaccggtgcagtgctcggtgcgatcgaaggcatcgtgcagttcgaccgcccggacaactacgtgcaaacactcaagccgcgcctggaagcgatcgatcagccggccgcgcaagccgccatcaaggacatcatcgcgcccaatgcgatgacctgggtgatcgtgggcgacctgaagaagatcgaagccccggtgcgcgcgctcaagctgggcgaggtgcaggtgctggacaccgacggcaagccggtcaagcgctgatcgcaatgaccggcacggccgcgcagcccccaaaggcaggcgcggccgatccgtgggaaccgataggcacaacgtgagcggcggcttctgcgagccgtccgcacgcgtccagacggtggcctgctggatctcggcagccgttagaacgccgagcattgcagccacatggcaatgcgcactgcagcgcgcgttaccgttagtgcatgatcgcgtttactgcctgcgtgcgcgcaggtgatatccacgccacgttatcggcctgcccgctagcgtggcgtccttccttttgctgacatggcgcattcaatgaaagacatgccgatggcagacgaagcgtcgcgccaacaggtgttggacggctatcgcatcgtcgacagcctgcccgaagacacctaccaggacatcgtgcaggtggccgcatcgctgtgcgagacgccgattgccctgatgtcgctggtggaccgcgaccgccagtggttcaaggcccgcctggggctggacctgcaacagaccgaccgcagccaggcggtgtgcggccacgcgattcgcagcccggaccacttgatggaagtgccggacctgagcaaggacccgcgcttcaccgatatttcggtggttaccggcgaggtgggcgcgcgtttctacgcgggcatgccactggtgacttccgacggcgtggcgctgggcacggtctgtgtgctggacaccgaaccgcgcgccctcaacgaggcccagcgcaccgggctgcaagcgctggcccgcgtcgccatgcgcctgctggacgaacgccagcgcgaactgcagcgcgaacgtgacgccatcctgcagccggccgctcctgcggcggcggcgggcaccgcgaccattaccgatgccgacaccggctatcacctggtgattctggaagtgcaggaactggccgccctggcccagcagcaaagcgagcgcctgctcggacgtgcgttgcaacagctcgaccagaccctggccagctttctgc

SEQ ID NO: 36 - DNA, 3287 bp, Xanthomonas campestris
  CDS (1001)..(2287)
agccggaacaggtcattgagatgtaccgcaacgacccccaactgatgagtgggctgcagagccgtgtgatggaggagcaggtgatcgactggatcgccgagcgcgccaagcacaccgagcagtcgctttcgttccaggacgcgattcgcgtttaacgcgaccgcgtggtacgagcccgcacccgcttagccagggtgcggatctcacacacgggcatcggcacaggccgctgccacaacaggaagactgatgagcattgtgactaaagccctgaacctggtgcccatggtggtcgagcagaccagccgtggcgagcgtgcgtatgacatctactcgcgtctgctgaaggagcgtctgatcttcctggtcggtccgatcgacgaccatatggccaacgtgatcgtggcgcagctgctgttcctggaagcggacaacccggaaaaggacatcagcatctacatcaattcgcccggtggcgtggtcaccgccggcatggcgatctacgacaccatgcagtacatcaagccggacgtgagcacgatctgcgtgggccaggcggcctcgatgggcgcgctgctgctggcatccggtgcggccggcaagcgctatgcgctgccgaattcgcgcgtgatgatccatcagccgctgggcggcttccagggccaggcgaccgatatcgacatccacgcccgcgagatcctgaccttgcgttcgcgcttgaacgagatcctcgccaagcacaccggccagtcgctggagaccatcgcgcgcgacaccgaacgcgacaacttcaagagcgcggtcgatgcacaggcttatggcctggtggatcaggtgctggaacgtcgtccggaagagtcgatccagccgtcttgagtcgcagacgcctgaaattgctggaaaaactggcagggtcgggctggacgaatgtctcccgaccctgtgctattctcgaaatcgaacccccgtgcatcgggtggggtaactgggtaacagaagcatgagcgaagaccgccaaggtcgttccggcgacagcaacaagattctctactgctcgttctgcggtaagagtcagcatgaggtccgcaagctgattgccggtcccagcgtattcatttgcgatgagtgcgtggagctgtgcaacgacatcattcgcgaggaactcgaagagaaggcgcagtcggcacgttccagcctgcccaagccgcgcgagatccttgaggtgctggaccagtacgtgatcggtcagctgcgcgccaagcgcacgctcgcggtggcggtgtacaaccactacaagcggatcgagagccggagcaagaacgacgaggtcgaacttgccaagtccaacatcctgctggtcggcccgaccggctcgggcaagacgctgctggccgaaacgctggcgcgcctgctcaatgtgccgttcacgattgccgatgccaccacgctgaccgaagccggctatgtcggcgaggacgtggaaaacatcatccagaagctgctgcagaagtgcgactacgacgtcgagaaagcgcagcagggcatcgtctacatcgatgaaatcgacaagatctcgcgcaagagcgaaaacccgtcgatcacccgcgacgtgtctggcgaaggcgtgcagcaggcgttgctgaagctgatcgaaggtacggtggcttcggtgccgccgcagggtgggcgtaagcatccgcagcaggaattcctgcaggtcgataccaagaacatcctgttcatctgcggcggcgcgtttgccgggctggacaaggtgatccaggcgcgttccaacgatgccggtggtatcggcttcggcgccaaggtcaagagcagcgagcgcaagcaggaagtcggcaagatcctggccgaagtcgagccggaagacctgatcaagttcggcttgatccccgagttcgtcggccgcctgccggtggtcgccacgctcgaggaactcgatgagccggcgctgatcaagatcctgaccgagccgaagaacgccatcaccaagcagttcaagaagctgttcgagatggaaagcgtggagctggagttccgtccggatgcactgtcggcgatcgccaagaaggcgctcaagcgcaagaccggcgcgcgtggcctgcgcaccatcgtggaatcggtgctgctggatacgatgtacgaactgccgtcacaggaaaacgtgagcaaggtggtggtggatgaatcggtgatcgaacacaagtccgagccgtatctgatctaccaggcccagccggctccggccaaggccgcgtctggcgactgaggcctcgcggcgatgccggcaggccgtggcctcgctggcggctaaagggtttccgaccgctacttgcaacacctcgccgatggccccataacggggccatcggctttttttacgccccaattcctcgatcccgcggagcgccccatggcccagtcccaaccagaaattctcgatctgccagtgttgccgctgcgcgacgtggtggtgtttccgcacatggtgattccgctgttcgtcggccgtgacaagtcgatgcgcgcgctggaaaaagccatggaggcggacaagcgcatcctgttggttgcgcagaagtcggccgagaccgacgacccggcagcgggcgatctgtacacggtcggcacgctcgcccaggtgctgcaactgctcaagctgcccgatggcacgatcaaggtgctggtcgaagggttgtcgcgcgtcaccgtcgacaaggtggcggaacaggacggcgccttgcagggccgcggcaccgaagtggaagccagcgatgcacgcgagccgcgtgagctggaggcgattgcgcgttcgctgatgtcgctgttcgaacagtacgtcaagaccaaccgcaagctgccgccggaattgctgcagaccctggccggcatcgatgagcccggacgtctggccgacaccattgccgcgcacatcggcgtgcgtttggctgataagcaacgcctgctcgaaatcaccgagatcggcgaccggctggagctgctggttggtctggtcgatggcgagatcgacgtgcagcagctggaaaagcgcatccgcggccgcgtcaagtcgcagatggaaaagagccagcgcgagtactacctcaacgaacagatgaaggcgatccagaaggaactgggcgatctggacgacgcgcccggcgaactggaagaactcgcccgcaagatcgccgaggccggcatgcccaagccggtcgagaccaaggccaaggccgaactcaacaagctcaagcagatgtcgccgatgt

SEQ ID NO: 37 - DNA, 2618 bp, Xanthomonas campestris
  CDS (1001)..(1618)
atcgaccagatgatcgaaaacctgcgtctgcagcgccgcacctggaacccggtcgagcgcggcgcgcaggtcggtgatctggtcgcgctggagacctggtcgcaggccggcaacgagcgtctgcccgctgaaggcgtggagaccggcagcagcgtgctgggctcgggcgtgatgttcgaccaaatcgaaaaaggcctggaaggcctgtccaagggcgaagacaaggccctgagcatcgatttcccggcggactggcgcgtgccgcagctggctggcaagacggtgcaggtgcacgtcaaggcggtcgaggtgtccgagccggtgctgccggaagtcaacaaggagttcatcaagagcttcggcgtgaagagtggcgatgccgagcagttccgcgccgacatccgcaccaatctggagcgcgagctcaagggtgcgctgatgaaccgtctgcgccgtgaagtgggcgagcaactgatcgccgcctacgcgcacgtggaaatgccgccgcgcctggtcgagaacgaagcccgttcgatgctggcgcaacaggtcgaccagatgcgtcgcagcggccgcaacccgggcgagatcccggccgatgcgcaccaggggttcatggacgccgctgccaaacgcgtgctggtaggcctgctggtgggcgaagtcgcacgtcgcaatgagctgcgcctggaatccaagcgtgtcagcgaaacgctgcgtctgatcgcctcgacctacgaggagccggaacaggtcattgagatgtaccgcaacgacccccaactgatgagtgggctgcagagccgtgtgatggaggagcaggtgatcgactggatcgccgagcgcgccaagcacaccgagcagtcgctttcgttccaggacgcgattcgcgtttaacgcgaccgcgtggtacgagcccgcacccgcttagccagggtgcggatctcacacacgggcatcggcacaggccgctgccacaacaggaagactgatgagcattgtgactaaagccctgaacctggtgcccatggtggtcgagcagaccagccgtggcgagcgtgcgtatgacatctactcgcgtctgctgaaggagcgtctgatcttcctggtcggtccgatcgacgaccatatggccaacgtgatcgtggcgcagctgctgttcctggaagcggacaacccggaaaaggacatcagcatctacatcaattcgcccggtggcgtggtcaccgccggcatggcgatctacgacaccatgcagtacatcaagccggacgtgagcacgatctgcgtgggccaggcggcctcgatgggcgcgctgctgctggcatccggtgcggccggcaagcgctatgcgctgccgaattcgcgcgtgatgatccatcagccgctgggcggcttccagggccaggcgaccgatatcgacatccacgcccgcgagatcctgaccttgcgttcgcgcttgaacgagatcctcgccaagcacaccggccagtcgctggagaccatcgcgcgcgacaccgaacgcgacaacttcaagagcgcggtcgatgcacaggcttatggcctggtggatcaggtgctggaacgtcgtccggaagagtcgatccagccgtcttgagtcgcagacgcctgaaattgctggaaaaactggcagggtcgggctggacgaatgtctcccgaccctgtgctattctcgaaatcgaacccccgtgcatcgggtggggtaactgggtaacagaagcatgagcgaagaccgccaaggtcgttccggcgacagcaacaagattctctactgctcgttctgcggtaagagtcagcatgaggtccgcaagctgattgccggtcccagcgtattcatttgcgatgagtgcgtggagctgtgcaacgacatcattcgcgaggaactcgaagagaaggcgcagtcggcacgttccagcctgcccaagccgcgcgagatccttgaggtgctggaccagtacgtgatcggtcagctgcgcgccaagcgcacgctcgcggtggcggtgtacaaccactacaagcggatcgagagccggagcaagaacgacgaggtcgaacttgccaagtccaacatcctgctggtcggcccgaccggctcgggcaagacgctgctggccgaaacgctggcgcgcctgctcaatgtgccgttcacgattgccgatgccaccacgctgaccgaagccggctatgtcggcgaggacgtggaaaacatcatccagaagctgctgcagaagtgcgactacgacgtcgagaaagcgcagcagggcatcgtctacatcgatgaaatcgacaagatctcgcgcaagagcgaaaacccgtcgatcacccgcgacgtgtctggcgaaggcgtgcagcaggcgttgctgaagctgatcgaaggtacggtggcttcggtgccgccgcagggtgggcgtaagcatccgcagcaggaattcctgcaggtcgataccaagaacatcctgttcatctgcggcggcgcgtttgccgggctggacaaggtgatccaggcgcgttccaacgatgccggtggtatcggcttcggcgccaaggtcaagagcagcgagcgcaagcaggaagtcggcaagatcctggccgaagtc

SEQ ID NO: 38 - DNA, 2576 bp, Xanthomonas campestris
  CDS (1001)..(1576)
gtcctggctgcattcggtgatggccagcggcccttccatggcttcgacgatctccaccagggtgatcgcgctggccgcacgtgcaagccggtagccgccgtgcacgccgcgcaatccttccaccaggccagcctgcgacaacggcttgaggatcttgctgacggtgggcgcttccaggccggcctgttcggccagttcgctcgcgctgagcacctggtcgctccgtgcagccagcaccgtcaagacaacggtggcgtaatcggtcaatttggtgacgcgcaacatgcgaagacggtgaatccctaaagcggaccgaaattgtactcttttgctccgctcagtccaaccgcgcggttcagccgccggaaaggccagcccgttgcggtggccggtgcgcttgtgcttggcgcgtgggcgcgctgcggtcacaatgagcggccccatcaccggaccctttcatgtcgcgcaaagttgccgcccgcaagtcacgtatccatggcaatggcgtgttcgccgtcgctgcgctcagcaagggcgaacgcatcatccagtacaaggggcgcctgcggacgcatgccgaagtggatgccgacgacaccggcgatgtggaaagcgggcacacctttctgtttacgctgagcgatgactacgtgctcgacgccaactacgaaggcaacatcgcgcgctggatcaaccacagctgcaatcccaattgcgaagcggtgatcgaagaggctgaaggcgacgaccgcagcaaggacaaggtcttcatcgaagccaagcgcgcaatcaagccgggccaggagttgacctataactacggcatcaccctgggcgagcggcacacgccgcgcttgaagaagatctgggaatgccgctgcggctcgaagaattgcaccggcaccatgttgcagcccaagcgctgagcaacggcgcgcggcggtcggcatgacgcggcgcgcacatcggttcacggatgcgcaacacgcgcattcctagggtgcaggctcctttgcagattgaggagttcctcatgacccattcgctctccggcaagaccgttgccgtgctcgcgaccagtggcttcgaacaatccgagctgcaggagcccaagcgcttgctggaatcctggggcgccaccgtcgaggtgatcgcgcccggcgacgatgcacagatccgtggctggaaccacaccgactggggcgacagcgtgccggtggacacgcccctcgcgcaggccaaacccgaccgctacgacgcgctcgtgctgccaggcggtgtgatcaacccggacaacctgcgcaccaacgcgcaggcgattgacttcatccgcagcgttgcggccagcggcaagccggtggctgcaatctgccacgggccgtggttgctggtggaaagtgggctggtgcgcgaccgcaaggtgacgtcgtggccgtcggtcaagaccgatctgagcaacgcgggcggccgctgggaggatgccgaggtggtagtggatggccagctgatcaccagccgcaagccggacgatattcctgcattcaccgatgcggttgccaaggcactggctgcctgaggtcacgcagcagctataaaagagagcccggtgcatcgctgcgccgggctcttttaatttcacgttggcagatgcgcggtgcgctcgcagcgccgatcggttacttgcctggcatggcgccggccggtaccagctgctcgatatcctggttgaagttcaccgccagccggccgttctggatcgccaccgactccacctgcacattgcccatcacggccgcaatggcgggatcgatcttgtagatcggctccttgcgtgcgtagtcggccaaccacgcgttgagcaactggcgcgtgctgctgtccaacttggcaccggcgttggcggggcggaaatcatcgaccgttggctggtcgagataaaacccctgctttgccgtgtcgtaacgcagcgcgctgctcaaggtgacgttgcccaccggcgccggcgcgccgccggccgtggccaatgcaaggtcgaacgccatcttcaagcggtcgccaggtggcaacgacagtgccgggttgctgaccgtcatttcgatcagcccacccagcgcatcgtgcgtctgcgggaagcgcccgcccaggtactgctgcacgtcggcggcctgcacgctgatctgccgcccctggatttgaggcgccgcccacgctgcggacggtggcatcgccagggcgagggcaagcagggatccggcaactaaacgcagtttcatcgggcaactccggcggtggatggcatcaccttagccagcgccggtgaaccacgggttagcgtgctgcggtgtggcgggatgagtacgacactgcgtgccgcggtggttggggtgctcgcagcgaatgcgcttcggtgttgtgggcgtcgactgcggatctcgcgtatcgccacttctgactacgaatgtccagttgcgtggacggcggctttaacgcagcaccggctgcagggtggccgaatagatttcccaggtgtcgctgccaacgcgtggctgcagacggtaagcaccgaccagccgttc

SEQ ID NO: 39 - DNA, 3977 bp, Xanthomonas campestris
  CDS (1001)..(2977)
cgcaggaagcactgcccggtgggcgctacccattggtaggtcccgatgaactgcgtcaggcgcacgtcccagccggtttcctccagcgtttcgcgcacggccgcctgcagcaggctttcctccggctccaggtgcccggcgggctggttcaacagcaggcgccccccgatggattcttccacctgcaaaaagcggccgtcacggaccaccacggtggccacggtgacatcggggtgccagcgagtttcttgaagcgtcatcagtcactgggtccaggaagtggcaggctggagattgatccgcgccgcacaggaaacaagcggcgggcgctaggtgcggcatccgcagtgcggcgcagtgcaacactgcatgccgccaatgcggatgccggcagtagcgcagtgcgccacggagcgtgccattgcaacgcagtggtggaagacgtcatcagtggccggccggagaacaggcgtaaattgtgcgggcgcggggccaaatcgtccaatgctgagggcgcgggaccgtataatggccggatgcctcgtaagacctcccacgaacacgaccacggcctagtggttgagaccagcaagcccgaggtggcgccaccgccgcgctaccaggtgctcctgctgaatgatgactacaccccgatggacttcgtggtgaccgtgctgcagcagttcttcaacctggagctggagcgtgccacccaggtgatgctgcatgtccatacgcgggggcgcggcgtctgcggcgtctacagccgcgaggttgccgaatccaaagtggcccaggtcaacgaattctcgcgcatgaaccagcacccgctgctgtgcacgatggagcaggcctgagcggtctggctggatcattccagcagtgcaacggtgttgctgcaatggcgtgctaacgccgtctgaacatgcctatccgatagggttgtggaaaatcccaacaaaagccgcatattgttggcaacagccgttcggagttaccaatgttcagcaaagacctagagcaaaccatcggccagtgctacaagcgagcccgtgaggcgcggcatgaattcatgaccgtcgaacacctgctgctctcgctgctggacaacccctccgcgcaagccgtcttgaaggcctgcggtgcggaccaggtgcgtctgcacactgacctggaacaggcgatcgaggcctcggtctcgcgcctggccgaggacgatggccgcgacacgcagcccaccctgggcttccagcgcgtgctgcaacgcgccgtctaccacgtgcagtcctccggcaagaaggaggtcaccggcgccaatgtgttggtggcgatcttcggcgaaaaagactcacatgcggtgtacttcctgaatcagcaggacatcacccgcctggatatcgtcaattacctctcccacggcatcgccaagctgggtgaagacggcgagcagccgtccgcctccgatggtgagcccaagagcgatgcgggcgagggcgaaggcaagggcgatgccttggccgagtacgccaccaacctgaacgatcatgcccgcaacggcaagatcgacccgctcgtcgggcgtgcggacgagatcgaacgcaccatccaggtcctgtgccgccgccgcaagaacaacccgctgtacgtgggcgaggccggcgtgggcaagaccgcgatcgccgaaggcctggccaagcgcatcgtcgatgccgacgtgccggaagtgctggctgacgcggtgatcttctcgctcgacctcggcgcactggtcgctggcaccaagtaccgcggcgacttcgaaaagcgtctcaagggcgtgctgacggcgctgaagaaggtgccgaacgcggtgctgttcatcgacgagatccacaccatcatcggtgcgggctcggcgtcgggcggcaccatggacgcctccaacctgatcaagccggccctggcgtcgggcgagctgcgctgcatcggctcgaccacgttccaggaataccgcggcatcttcgagaaggaccgtgccctggcacgccgcttccagaagatcgacattgtcgagccgaccgtcggtgaaaccttcgaaatcctgcagggcctcaagcccaagtacgaagcgcatcacggcgtgacctatgccgacgacgcgttgcaggcggcggtggatctgtcggtcaagcacatcggcgaccgtctgctgccggacaaggcgatcgacgtgatcgacgaagccggtgcgcgccagcgtctgttgcccgaagggcagcgcaaggagctcatcgacatcgaggagatcgagaccatcgtggcccaagatggcgcgtattccggccaagcaggtcagcgcgaccgacaaggacgtgctgcagcatctggaacgcaatctcaagatggtgatcttcgggcagaacccggcgatcgaaacgctggctggctcgatcaagctggcgcgctcgggcctggccaatccggagaagccgatcggcaacttcctgttcgctggcccgaccggtgtgggcaagaccgaggtcaccaagcagctcgcgctgcaattgggcatcgagctggtgcggttcgacatgtccgagtacatggaagcgcattcgatcagccgtctgatcggtgcgcctccgggctatgtcggtttcgatcagggcggtctgctgaccgaaaagatcgtcaagacgccgcactgcgtgctgctgctggacgaggtggagaaggcgcatccggacatcttcaacatcctgttgcaggtcatggaccgcggcatcctcaccgataccaacgggcgcgaagcgaacttcaagaacgtgatcctggtgatgacgaccaacgccggtgccacgcaggcctcgcgtcgttcgatcggtttcaccaagcaggatcattccaccgatgcgatggaatcgatccgccgcggtttcacgccggagttccgcaaccgtctggatgcgatcgtgcagttccagccgctgggctttgaccacatcctgcgggtggtggacaagttcatcatcgagctggaaatgctgttgcaggaaaagcacgtgtcgctgtcggccacgccgaccgcgcgcgactggctggcccagcatggcttcgatccgctgatgggtgcgcgcccgatgtcgcgcgtgatccaggagaagatcaagcgcccgctggccgacgagttgctgttcggcaagctggtggaaggtggccgcgtcaatatcgacgtcaaggatggcgagctggtggtcgaagcgcatccggaaccggagcgcctgttgccggctacggttgattgatcgttgcgattggtcgctgctagagaaaaagccgctcgaaagagcggcttttttgtgttgaaggcgctggagctggagcggcaccggtctggcgtactgctggagcgttggggcgggtgccggcgcatcacgggtgcccaagtccgggaggatcgccgtaaaccaccgagacgccatgcgcttcttacaaccccgagccgcgctgcagtggtcgaacaatcacatgatctgcgggtgcatcacgaagctgtgctatcggccgccgaaaacgtcaacgcacagatcagcgcgcaggcgaaagcctgcccatgtaacgcgaccgcacgggcagaaaataaaccccaggcacgacaaaaagcggcacttggccgcttcgcgtcttgataccagaaccgcttacttcatgcggtaggtaatacgacccttggtgaggtcgtacggtgtcatctcaactttgacacggtcgccggtgaggatgcgaatgtagttcttgcgcatacggccggagatgtgggcgatgatttcatgcccgttttccagcttgatccggaacgtggtgttggggagcgtctcgctgacgctgccttcgaattcaatggagtcgtctttcgacatgtaatcctgtgcaatcacggttggcccggcagggccgtaagggacagcattttacgccccatctcgccgaggcgcaaa

SEQ ID NO: 40 - DNA, 2978 bp, Xanthomonas campestris
  CDS (1001)..(1978)
accggcgccaatgtgttggtggcgatcttcggcgaaaaagactcacatgcggtgtacttcctgaatcagcaggacatcacccgcctggatatcgtcaattacctctcccacggcatcgccaagctgggtgaagacggcgagcagccgtccgcctccgatggtgagcccaagagcgatgcgggcgagggcgaaggcaagggcgatgccttggccgagtacgccaccaacctgaacgatcatgcccgcaacggcaagatcgacccgctcgtcgggcgtgcggacgagatcgaacgcaccatccaggtcctgtgccgccgccgcaagaacaacccgctgtacgtgggcgaggccggcgtgggcaagaccgcgatcgccgaaggcctggccaagcgcatcgtcgatgccgacgtgccggaagtgctggctgacgcggtgatcttctcgctcgacctcggcgcactggtcgctggcaccaagtaccgcggcgacttcgaaaagcgtctcaagggcgtgctgacggcgctgaagaaggtgccgaacgcggtgctgttcatcgacgagatccacaccatcatcggtgcgggctcggcgtcgggcggcaccatggacgcctccaacctgatcaagccggccctggcgtcgggcgagctgcgctgcatcggctcgaccacgttccaggaataccgcggcatcttcgagaaggaccgtgccctggcacgccgcttccagaagatcgacattgtcgagccgaccgtcggtgaaaccttcgaaatcctgcagggcctcaagcccaagtacgaagcgcatcacggcgtgacctatgccgacgacgcgttgcaggcggcggtggatctgtcggtcaagcacatcggcgaccgtctgctgccggacaaggcgatcgacgtgatcgacgaagccggtgcgcgccagcgtctgttgcccgaagggcagcgcaaggagctcatcgacatcgaggagatcgagaccatcgtggcccaagatggcgcgtattccggccaagcaggtcagcgcgaccgacaaggacgtgctgcagcatctggaacgcaatctcaagatggtgatcttcgggcagaacccggcgatcgaaacgctggctggctcgatcaagctggcgcgctcgggcctggccaatccggagaagccgatcggcaacttcctgttcgctggcccgaccggtgtgggcaagaccgaggtcaccaagcagctcgcgctgcaattgggcatcgagctggtgcggttcgacatgtccgagtacatggaagcgcattcgatcagccgtctgatcggtgcgcctccgggctatgtcggtttcgatcagggcggtctgctgaccgaaaagatcgtcaagacgccgcactgcgtgctgctgctggacgaggtggagaaggcgcatccggacatcttcaacatcctgttgcaggtcatggaccgcggcatcctcaccgataccaacgggcgcgaagcgaacttcaagaacgtgatcctggtgatgacgaccaacgccggtgccacgcaggcctcgcgtcgttcgatcggtttcaccaagcaggatcattccaccgatgcgatggaatcgatccgccgcggtttcacgccggagttccgcaaccgtctggatgcgatcgtgcagttccagccgctgggctttgaccacatcctgcgggtggtggacaagttcatcatcgagctggaaatgctgttgcaggaaaagcacgtgtcgctgtcggccacgccgaccgcgcgcgactggctggcccagcatggcttcgatccgctgatgggtgcgcgcccgatgtcgcgcgtgatccaggagaagatcaagcgcccgctggccgacgagttgctgttcggcaagctggtggaaggtggccgcgtcaatatcgacgtcaaggatggcgagctggtggtcgaagcgcatccggaaccggagcgcctgttgccggctacggttgattgatcgttgcgattggtcgctgctagagaaaaagccgctcgaaagagcggcttttttgtgttgaaggcgctggagctggagcggcaccggtctggcgtactgctggagcgttggggcgggtgccggcgcatcacgggtgcccaagtccgggaggatcgccgtaaaccaccgagacgccatgcgcttcttacaaccccgagccgcgctgcagtggtcgaacaatcacatgatctgcgggtgcatcacgaagctgtgctatcggccgccgaaaacgtcaacgcacagatcagcgcgcaggcgaaagcctgcccatgtaacgcgaccgcacgggcagaaaataaaccccaggcacgacaaaaagcggcacttggccgcttcgcgtcttgataccagaaccgcttacttcatgcggtaggtaatacgacccttggtgaggtcgtacggtgtcatctcaactttgacacggtcgccggtgaggatgcgaatgtagttcttgcgcatacggccggagatgtgggcgatgatttcatgcccgttttccagcttgatccggaacgtggtgttggggagcgtctcgctgacgctgccttcgaattcaatggagtcgtctttcgacatgtaatcctgtgcaatcacggttggcccggcagggccgtaagggacagcattttacgccccatctcgccgaggcgcaaattttatgttaagcggactcctgcaagcccgcttgcgtgccgttcgccatagcgttgcgcccagctgccaggtggctctggcaatgcgacctgggtggctacatgctgcaggaactgctcgcgtggcaggcgttgggcgcccaggcgcatcaggtgcgggttttctatctgcgcatcgatcagcggccagccgcggccatgcagctcggcggcgagcgctgccagtgcaatcttggagccgccactggcgccactgaacatgctttcgccgaagaacatctggccgatcgcaaccccatagatgccgc

SEQ ID NO: 41 - DNA, 3353 bp, Xanthomonas campestris
  CDS (1001)..(2353)
aacagcgtgccgagcgcgaaaaagccctgggccaggatgccaaggcgctggagcagttgctggccaatctgcgtgcagccgcggccaaggccgacgccgagcgccgcgcggcggccaagcgtgcggccgccgaagccgccgcgcaggccaagcgcggcaaaaccgatcgcccggaacgccccggcaagactccgcccaaggtcgtggccaacgcaccggcgccaaaggtcggtggcctgagttggccggtgtcgggcaatctgctggcgcgcttcaacgccacgctgccggatggccataccagcaagggcgtgctgatcggcgcgcccaagggcagcacggtcaccgcggtggccgatggcacggtggtgttttccgactggatgaccggctacggcatgatcctgatcgtggatcacggcaatggctacatgagcctgtacgcgcacaacgacaccttgctgcgcgatgccggcgccagcatcaaacgtggcgaagccgtggccaaggtcggtagttcgggcgggcagggtgtgccggcgctgtacttcgaactgcgtcgcaacggccagccagtggatccctcgagctggctgcagcgccgctgagtgcatggggcgccgcacttatccggcgtctgcttgctgcatggtgttgccgctcacatgccggcagttcgcggcgccagagtgtcgctgcggactgtcccgacggcagcaccgcagcagatctcatcgcgcagccttgccggtgcttcgcacatacgccggcgttgacggcgattcaacgcggatttaccgctgcttcgcgcataatccagccagcgccgcgctgtggggcgtcttctctcttttcggagtgcttcatgcgcgtagccgtcctgtccgttgccctgtcgctggccctgttcgcgtcgcccggttgggcacagcaggccagccccgctgccgcaccggccaccgcggacgacccggaagccaacgaagccgccgtctccaaggtgccgctggacgagatccgccgcttcgtggccgtgtacaacgcggtcaagcaggcctacgtggacccggtcgaggacaagaagctgatgcatgcggccgtgcgcggcctgctgtcggacctggatccacacagcacctacttcgacaaggaagatgccgaggccttcgacgaacaggccaccggcgcctatgacggcatcggcgtggaactgctgcagcagcaggacaacacgctcaaggtgattgcgccgatcgacgacacgccggctgcacgcgccggggtcaaggcgggcgatgtgatcgtggccatcgacggcaagccgatcgacgccagcaaggcaatggagccgctgcgcggcgaatccggcagcaaggtcaccctgaccatcgtgcgtgacaaagtcgccaagccgttcgacatcaccctgcagcgcgaaaccattcgcgtggccagcgtgcgcagcaagctgctcgaacccggctatggctacatccgcatcagcaccttccaggccgacaccggcgccgacttccagaagaacctcaagcaactgcaggccggcggcaagctgcgtggcctggtgctggacctgcgcagcaacccgggcggcctgctgacctcggccgtgcaggtggccgacgatctgctcgacaagggcaacatcgtcagcacccgcgggcgcatctccatcagcgacgccaagttcgacgcaaccccgggcgacctgctcggcggtgcgccggtggtggtgctggtggacgccggctcggcaagcgcgtcggaagtgttggccggcgcgctgcgcgacaaccagcgcgcgcgcatcatcggcagccgcaccttcggcaagggctcggtgcagaccgtgttgccgctggacaacggcgactcggtcaagctcaccaccgcgcgctattacacgcccagtggcaagtcgatccaggccagcggcatcgtgcccgaagtgatgctcacccccgaaccgcagccgggcgacgccgacgtgccggccagcctcaccgacttcagtgaggcgaccctgccgggccacctgcgcggcgatgcagaaggcgaggagggctatagcgccggcgacgtgctgccgggcgatggcccgatcaacgaggcgctggccgaactcaagcaacctggctcagtcgccaaggcacaggccgcgcgcaaggccaaggcgcaagcgcaaaagcccaaggccatcaagccaacgcccgagcccaagccggccgcaccacgcccgcccgccagcgagcagaccacgccggccgagccgacggacaaggccaagccagcggcgccagtaccggcagctgtgccggccgagccggtcaagtaaagggcagtgctgcgggtgccacgtgcacccgcagtgcgcagccggcgctggtgtgcatgcgcacgtcgtgcgttgtaagtcgtgccgcgaatttacatgcagcagttgttagccacgcatgcggtgattggacgcagacgttgcggaatagaggcacgtctcgtaggcatcgccggtcgccaggcgacggctatttgctttgaaaaactctctgcaagatgtcggcgctttttcgcaggtggtcttccacgtttggtcgcactcacttgctgatgtgcgcgatacgtagggactccgcgcactgcacgtcattaaacgtgtagaacgtctttagcacgtcttcgccagcagcctagacgctgggcgagggcacctagcctaagcactctcccagcgacgcagttgcgcgttgaggttgcgcgttgaaaacaccacttcatcactaacgatgctctgccggctttttgcgggtgccgcgctgctcgatgcagtgattcgcgctcaccacctaagctgggtgacatggaatgcacgcactgcatatcgcgcgaatgcgtcaccaaaatcgatgcgcagcgcagcagcacgaccagtccggataacgcaggccgcaggtgcgcgtgatgcgtgccacgctgccaccaatacggcgggttgaaaacctggccgttgtgtcggtcatgcgtgctccctgcacgtcattgcaggtggccagcaagacatgcggtcgtgcctcttgggcgcactgtgcatcgcaggaccacgacgcgtccttgcgggaagccgcatggacacatacggacttgttgcagctgttttcgctacatcgttctagaagagcaggtgatgacaacccgttcgcgcatcagacatcggcagcggcagcgcagcagcaatccgcaacgcaaccacgcaacatcacaccctaacgcgcaaccagcaacgcctcggggttgaccagattgcgcggtgtgccagcagcgaacgccagcacgttgtc

SEQ ID NO: 42 - DNA, 4184 bp, Xanthomonas campestris
  CDS (1001)..(3184)
gtctgcgaagaagccagctgcccgaacatccacgagtgcttcagccacggcaccgccactttcatgatcctgggtgaggtgtgcacgcggcgctgctcgttctgcgacgtggcgcatggccggcccaagccgccggatgcgagcgagccgaccagtctggctaccaccgtggccgacatgggcctgaagtatgtggtggtgaccagcgtggaccgcgacgacctgcgcgacggcggtgcccagcacttcgtcgactgcatctcggcgatccgcgccagcgcgcccaagacccggatcgagatcctgaccccggacttccgtggcaagggccgcatggaccgcgcgctggagatcctggccaccagcccgccggatgtgttcaaccacaacatcgaaaccgtgccggacctgtacccgaacgtgcgccccggtgccgattaccagtggtcgctgacgctgctgcagcgcttcaaggcgcagcacccgaccatcgccaccaagtccggcatcatgctcggcctgggcgaaacgatggagcaggtgcaggccaccttgcgcgacctgcgcgcgcacgatgtggacatgatcacgatcggccagtacctgcagcccacgccgcaccaccacccggtgatgcgctactggacgccggaggaatacaaggcgctggaggactacggcaatgcgctgggcttcagccatgtggcgtccggcccgatggtgcgttcctcgtaccacgccgaccgtcaggctgccggtgccggcgtcgccgcctgagcccaaccgcatcggtgtgtgcatcacggccctgcgcagtgcgcggggccgtgtcgtatccgggccagcgtgccaagggtacgggcgcgttcacaatctgctacatgcccggagctaggctgattcagccagcggatgacaacgccagcaactttcggcactgtcatgctgtcacatccgatcaccgcccttctttcatcggccccgtgccgagagccattcgatgacctacaacgtttctgcgtccctgaaggccggcctgctggcactggtgctcaccactccgatggcattgctggcgcgtgcggacacggcgctgcctgccgcggccacgcccgaacaggcgacggcgaccaaactggtctacgggctgctctcggacagccggtatgcgtaccgcccgcgtaccctcgacgaggcgatgtccaaggacgtcttcaagcggtatctggagacactggacggcggcaagcaattcttcacccaggccgatgtggatgcgttcgcgccgctgcaggcgggcgtgggtgatgcgctgcgcggcggcaacctggagccggcgttccaggtgttctcggtctacaagaagcgtgtcgatcagcgcgtcaaatacgcgcgcgaactgctcaagcaggatttcgatttcaccggcagcgagaagttcgagtacgaccgcaaggacgtgccgtgggcagcggacgacaagcagctggacgtgctgtggcggcagtcggtgatgaacgactggctgcgcctgaagctggccggcaagaagtcggacgacatccgcaagacgctggacaagcgctatgccacgttggccgattcggtcaacgaactgaaggccgaggacgcgttccagttcttcctcaacgcctacaccaatgcggttgacccgcacaccgattacttcactccgcgcaccgccgagaacttcaaccagcagatgtcgctgtcgctggaaggcatcggcgcgcagctgcagaagcaggacgacatggtggtgatccgcgaggtgatcccgggtggtccggctgcggtggacggcacgctcaagccgggtgaccgcatcgttggcgtcggccagaccaagagcggcgccatcgaagatgtgatcggctggcgcatcgacgacgtggtggccaagatccgcggcagcaaggacacccaggtgcgcctggagtacatcccggccgagctgggcatcgacggcacgcaccgcaccgtgaccctgacgcggcagaaggtgcgcctggccgaacaggccgccaagggcgagaccatcacgctgccggccagtggcgacgtgccgcagcgccggatcggcatcatcaaactgccgggtttctaccaggatttcgaaggccgtcgccgtaacgcggccgactatgcctcggccacccgcgatgtcgccaagctgctggcgggtttcaagtccgacaaggtcgacggcgtggtgctggatctgcgcaacaacggtggcggttcgctggacgaagcgatcgagctcaccgggctgttcatcgagcagggcccggtggtgcaggtgcgcgaatccggtggccgggtcaccgtcaatggcgacagcaacccgaaggtggcctgggatggtccgctgggcgtgctgatcaaccgcggttcggcttcggcgtcggaaatttttgctggtgccatccaggactacggccgtggcctggtcattggtgaaaccaccttcggcaagggcacggtgcagaacattgttgacctggaccgctggccggccgccgaagggcagcgttatggccaggtcaagctgaccatcgcgcagttcttccgcgtcagcggctccagcacccagcacaagggcgtggtgccggacatcgccttcccggccagcgtggatgccaccgagttcggcgaaagcacctacgacaacgcactgccgtggacgcgcattgccgccgtgccgcacacccagtacggcaacttcgcccccctgctgccgaagctgcagaccctgcacgccacgcgcatcgccagcgacaaggaattccagtggtgggaagaggacgtcaagcagttccgtgacgagaaggccaagaagtacatttcgttgaacgaagccgagcgcgtggccgaacggcagaagcaggatcagcagcgcaaggaccgccagcaggtgcgcaagcagctgggtctgccgctggacccgctggccgacgacagcgacgacggcctgaccggcaacgagcgtgacatcgtcaaggacaccgcacgcgagaaggccgctgaaaagcgtccggatccgctgttgcacgaatcggcggccatcctggccgatgcgctggggttgttgtccaaggaccagccgctgtcggcgcaggtgctgccgcaatcgacggcaccgggtcgctgggctgactgattgcttgcgtcattgatgggtgagcaacgccaccgcttgtcggtggcgtttgtttttggggcgcatcggttggtgacgtgtgcgcacccgactgcccgcagcagatcaggccagcgcctgccagcaggcctgcaggccgagcagcagcaaggtgccgaagaacacgcgccggaacatctcggcggagatacgtcgccgcacccgggtgccggcccacacgcccagcgctgttggcagcagcgccagtagcgatggcacgcccgccgatagcggcacgccgccctgccagaccagcgcgatggccagggcaagcgttgcggtaccaaaacaggtggccagtgcgcggatcaacagttcgcgtggtaaccccagcgcggcgaaatacggcgctatcggcagcactaccacgccagtggcgccgttgagcaggccgctgatcaggccggtcaatgggctggcccagcgttcatggcgtggtggcaacgtccactgccagcgggaaagccccagcaacgcataagccaccaacaacacgccgagcgcggcacgcagggtgcgcgggtcgccaccgatgatcaacccggcagttagccaggtgcccacgctcatcagtgccagcaggggccatagtcgccgcaacagtgcacccggtgccggcccccaggcctgctgcagattcgtcaccagcgaaggcagcaccaacaacgcggctgcctggctgggtggcagccacagctcgagcaggcccatggccacggtcggcaggcccatgccggccacacccttgaccccgccagccagcaggaacacgcccgcgatcaagagccagtgcaatgcatcgtcattcatgccgggcaggatcctgccgccggggcgaggcggcaagctgtctttgtttgaggcagacttcgtccacaccgaaggctggggctgcgcgatgcaagtggagttcgtcgatctgcggattttcgtggcagtggccgacaccggcagcatcacc

SEQ ID NO: 43 - DNA, 4412 bp, Xanthomonas campestris
  CDS (1001)..(3412)
cggcacggtcaagaagaccccgctgagtgaattcgcgttccgtctggcgcgcggcaagatcgccatcaatctcgatgaaggcgatgcgctggtgggtgtggcgctgaccgatggcgaccgcgatgtcctgctgtttgcttccaacggcaagaccgtgcgctttggcgagtcgaccgtgcgctcgatgggccgtaccgccactggcgtgcgcggtatccgtctggccaagggcgaggaggtggtcagcctgatcgtttccgagcgtgccggcggagtggaagacgaggtcgaggacgagagtgccgaggaggtcgtggagaccaccgacggcgccgagccggcagtgatcgatgtcgccgacaacggcgatgtggcctacatcctgaccgccaccgagaacgggtatggcaagcgcacgccgctggccgaatacccgcgcaagggacgcggcacgcaaggcgtgatcggtatccagaccaccgagcgcaacggcaagttggtgcgtgcggtgctgctgggttccaccgatgaagtgctgctgatatccgatggtggcaccttggtgcgcacgcgtggttcggagatttcgcgcgtgggccgcaacacccagggcgtgacgctgatccggctgtccaagggcgagaaactgcaggccgtcgaacgcctggatgcctcgctggaagagcccgaggatgtggtggacgaggcggttgcaatcaccagcgatgcgccgcctgccgagggctgatcgcgcggtgcgatgtgtgcagtcatgaaaaacgccggcgcattgccggcgtttttttttgcggtgtttggctgtaacgcattgttgacagaacatgtcagcaaagaacgcaggtctttcacaaaaacgggtcgaaggtagttcgcggtgcggcaacgttgcgttggccgcagatgcaaagaggaactactgatgtcgacattgcttcgtccctcccgtcgcggccattgggccctggcgattctgggaggcgtgattgtgctgctggggctggtgctgctggcaggtggtgcgtggctcgccacgctgggcggatcctggtattacgcaccggcaggcctggcgatgctgatcgctggcgtgttgctgttccgcggccgcaatgccggcgcgtggtggtttgccgccgtcgtggccgccacgctgctgtggacgtggtgggaatcgggcagtgattactggcgctgggtgccgcggctcgggttgatcgtgggcctggcgtttgtgctggcgctgctgttgcccaagctcgagcgcccggtgtcgcgcgccgtctcgcgcagcgtggctggcggactggcagtggtgtttgtggtggcctttgcgttggccttcgtgccgttcggaatcaccgaggccgagggtgcattgacgcaggtggcaggcatcgccgcaggtgtggtcaatcgccccgccgatggcgcacagccggcacagcccgccaatgcgccggtcgatgacgactgggccgcatatggacgcagccaggctgggcagcgctattcgccactcacccaaatcaatcgcgacaatgtcagccagctgcagcaggcgtgggtgttccataccggcgatgtgccgaccaagcgctggggcgccgaaaccacaccgttgaaggtcggcgacagcctgtatctgtgtagcgcgcgcaatcaggtgatcgcgctggacgcctccaccggcaagcagcgctggcgctacgaccccaaggtcaaggacgaggccattccatacaccgcggcctgccgcggggtgtcgtattacgaagtgccgacggcggcgccggccgcaaatgacgcagccgcttcgcaggcggcgacggcgcctgcgctgtgccgtacgcgcgtgatcgaaggcacgctggatgggcgcctgattgcattggatgcgcgcaccgggacaccatgcgcggacttcggcactcacggtcaggtcgacatcaccgtcggcatgggcgagacgccaccgggctacatctcgatcaactcgccgccggccatcgtgcgcggcgtggtggtcatcgggcatcaggtccttgacgggcaaaagcgttatgagccctcgggcgtgatccagggctttgacgcggtcaccggccagttgcgctgggcctgggacatgacccatcccgaatggaatggtgcaccgccgcctggccagacctggacgcgcggcacccccaacatgtggaccaccgcagcggccgacgagcagctgggttacgtgtatctgccgatgggcaattccaccgcggactactggagcagctcgcgcacgccgccggaaaaccgctatgccacctcgctggtggcgttggatgtgaccaccggcaagccggtgtggaatttccagaccacgcatatcgacgcatgggattacgacctcggctcgcagcccagcctgatcgatttccccaaggatggcgtcaacgtgccggccgtgttgctgccgagcaagcagggcgagttgtacgtgctggaccgtcgcaccggcgagccgctggttggcgtggaagagcgcgccgtgcccggcggtggtgtggagccgcagatgcgctacaagacccagccgttctcgctgtatcacacgctgcgcaagccggatctgaccgagcgcgacatgtggggcatgaccccgatcgatcagctggtgtgccgtatccagttccgcaaggcgagctacaagggcatctacacgccgcccgaagccgaccggcattccatcgaataccccggctataacggcggctcggactggggcagcgtggcggtggatccgcaccgtggcgtgatcgtggccaactacaacgacatgcccaactacaacatcctggtgccgcgcgccaaggcggacaaactgggctgggctccgcgcgatcaggtgcgtggcgatgccggtggtgccgaaggcgccggcgacccgcaggccggcacgccctacgcggtaaacgtcaacgccggctggcgtttgccgttcaccaagctgctgtgcaagcagccaccctatggcggcattcgcgccatcgatctgaagagcggcaagaccctgtgggaccgtccctttggcagtgcgcgcggcaacgggccgttcgggattcgctccggcctgccgatcgaaatcggcacgccgaacaacggtggctcggtggtcactgcgggtggtctgatcttcattgccgccgcaaccgatgacctgatccgtgcgatcgacctggccaccggcaaggagctgtggcacgccaagctgccggccggtggccaggccaatccgatggtctacgcgtacggcggccgcgagtatctggtgatcatggcggccggtcaccacttcatggaaacaccggcaggcgatgccctggtggcctacgcgttgccgcagcggtaagcggcaacgcggttcaatggatctgtgaatcagaaacgcgccgcttgcggcgcgtttctttagcacgcattcctcgcaacccgtccagactcgcagctcgaatagtcagcccgagcgctcaattcaaggcacgatacgaatggccggcgcgactgctttgtaagaaagcaaacgtcccacattccgcgccgcacgcgcctcaggcactcaacccaggcactcagttcacagcacttgcaagactgtcttgctcttccttcacggcatcctgcacgtcctgcgccagcgtggccttgacgataatcgagcgaatggcgtcggtggcctctgacagcggtatgtcgctgctcaggcgctggaaggtatcgcggctgttatagccggaaccatccttgaggtagtgctcgtacatcgacagcagatcctggcacgccgtgatcagcgcagccacattgccacgcagcagcgcatggccgatctggtcgaagctgtgcagacaatcggccagccaatgcaggtcgtagcgtgcagtgctgggttcgggcgcgttgtggccgcggctgtaagcgctgtaacggcgcaggctgcgtgccacgcgcgcgacatgcgcaaacagcgtgccgatttccgcagcgatatccaggcgctgcaccatcaccatcggaatcacctggtcgggcgtgtctgaagactgctgcgccgaggcattctgttcctgctgatcgagcgattgcttcaacaccgtgtagctgcgttgcagggactccagctccagcgtggtcgccgccgcacgtgctttcagctgcgacagcgccgcctgatagcgcgcaagactgtcctgggtgctggcgtgctgcccgtactcggcagtgagctggcgcccacgcacgaacgccaaccccgtggcgatgagcgcccagagtgcgaaggcaggcaccaggtagatcaggatgcccatggaggctcctcggaacgatgcatatcttgtcggccaggaatc

SEQ ID NO: 44 - protein, 486 aa, Xanthomonas campestris
MRASTDQETHPMHATSRPCPRTFWQRAHQLLLIALTLLLTTASAQADVILHAFNWPYATVEARAKQIADAGYRKVLVAPAYRSEGSAWWARYQPQDIRLIDNPLGDTTAFARMVQALANNGVETYADVVFNHMANEAATRSDLNYPGSAVLAQYAANPGRYDALRLFGTVQSNFLSGSDFGPAQCISNYNDAFQVRNYRICGGGSDPGLPDLLGNDWVVQQQRAYLQALKGLGVTGFRVDAAKHMTFDHLNRVFDAGIRSGVYVFGEVITGGGSGNGDYDQFLAPYLQSTPHAAYDFPLFNAVRTAFGVGASMQQLVDPASAGQALPGNRAVTFAVTHDIPNNAGFRYAILDPVDETLAYAYLLGRNGGVPMVYTDNNESGDNRWVNAYLRDDLRRMIGFHNGVQGSDMQVLSSSACHILFRRGSLGIVGINKCGNPVNTTVAMNGSVLFWNADYVDALGSGTVVRISSGSYTFTLPARQARMWRR

SEQ ID NO: 45 - protein, 570 aa, Xanthomonas campestris
VRGAGPKRPGRAMMRSVLMAAMLLYSGAACAAPAPGDYYGTLEPFAADAVYFVVTDRFVNGDTGNDHRDQGGAHRSFDVPTPCDGGVGDNIGYLGGDFKGIVDHADYIRGLGFGAVWITPIVDNPDEAFTGGKPITCESTLSDHGKTGYHGYWGVNFYRLDEHLPSPGLDFAGFTRAMHANDLKVVLDIVGNHGSPAYSMPVAQPGFGKLYDAQGRLVADHQNLAPAQLDPAHNPLHAFYNTSGGLAELSDLNEDNPAVLDYLAGAYLQWMEQGADAFRIDTIGWMPDRFWHAFVARIREKRPGVFMFGEAFDYDPAKIAGHTWARNAGVSVLDFPLKQQLSAVFGHKQAGFEQLATPLYLRKGPYGNPYELMSFYDNHDMARLDASDTGFIDAHNWLFTARGIPVIYYGSETGFMRGRAEHAGNRNYFGEERVSNAPQSPIFGPLQRIATLRRNTPALQRGVQVDLQLRGNQAAFLRVYQHAGMTQTALVLLNKGDAAADIAVSRLLQPGSWRDAFSGEQVQVQGRVTLQVPAHGVRVLLSDAPVTDVALRKQLDAQMADQAARDARNK

SEQ ID NO: 46 - protein, 319 aa, Xanthomonas campestris
LLAGATRVLAQDRHLRLLAAVLARLHIERVVRLPPRAGRPPGPAQPHVAVLFHVAQHAFQRVVADRIVGRAELAQEAFLLVLAQPVGKAAQLVCAHRGTGAFGLGAAVVAVQVLMRFEGQIVAGVLENLQGLGIGDAVAGAAPAVRAGVGRARHQNQIVRAGLADGVDRRLCGRGPVGGGDAIGLVHQPEDHLLVVFELQRQAAPQIGQCCRRHLVGADALSVVLGVVVQVQDQELLVGLGGGHGFFDQRQLRRIQLAIEGWLQALPQERQANGVHALAGEIAEIRRRRVGVVLVERAGLLARVELGAGDVDAHVLQSA

SEQ ID NO: 47 - protein, 555 aa, Xanthomonas campestris
VRRRSGELCRQLVGLVFFLSTALPSIARGAGGMIAMIDPPTKRRTTTTGAWLAVLSLLLLLFSTPSVRAAEPATSGPYQWRSVAIGGGGFVTGVLFHPAERGLAYARTDVGGAYRWDAQAQQWTALTDWLGADDWNLMGIDAFAVDPADADALYLAAGTYMHERAGTAAVLRSFNRGRTFERADLPFKLGGNQLGRANGERLAVDPHDGRVLLLGSRDAGLWRSDDRGAHWAKVASFPDAALAGATARNHVGREQAVGIAFVVFDAASGNNGSPTPRIYVGVSTEQTSLYVSEDAGRSWAPVAGQPRGLRPSHMAGGSDGHWYLSYGDQPGPDLMAGGALWKFTPAQGRWREISPIPQPASGDGFGWGAVAVDPQHPQVLLASTFRRRTPRDELYRSVDGGKHWTPLLADAVFDHSAAPWTAHATPHWMGALAIDPFDGNHALFVTGYGIWASRNLQDFAAPQRPLQWWFQDRGLEETVPLDLLSPMAGAHLLSALGDIDGFRHDDLDRVQLQYAGPRLTNGESIDAAGQAPQWVVRSGTVRARRNNEIRALYYA

SEQ ID NO: 48 - protein, 582 aa, Xanthomonas campestris
IALATLIPVTAPAMQVSTQAPLVDATGQTLHIRGVTWPGFDRAGLAAVGMRNNTLAQLLDRMQASDINAVRVPVCAAVLQRAPVAAAEVAGDSTLRGLDSLQLLDAVVHAASQRGMQVMFAFADGGCDDRAPLLGAQQQAWTRGLVTLARRYGGNANVLGIDLGSSGYRNASWAGNAADQDWNRVASRAVARVLAQAPRWVVGVEGVGSNAVCSDPERKAPGSNLQPFACVPLDIARRHLVLMPKLAGPDRDTTDAFAAPGFAQALPAMWQRDFGQFAIDHAVVPVSLGGGLGDGDPRDPAWQTALSGYLANAGIRSAFLGSWETGNANNGGLLAPDGSPRADKLLILRHAWGRLPVMPAIATATGDSTKNASGKKPWNSTFTGTATVTGSGYSGGALLLDPIPSDAFITALNPVQLNFGGVKAALAGAYLQVNGPKGTTTVYVTDLYPEGASGGLDLSHNAFAAIGDMVQGRIPISWKVVRAPVTGNLQYRIKEGSSRWWAAIQVRNHAYPVVKLEVKQGSTWKNLQKMDYNHFLGEQLGNQPLTLRITDIRGKVLTDTLPRLPEDGSKPAYFEPGHVQFP

SEQ ID NO: 49 - protein, 555 aa, Xanthomonas campestris
TSKLAVSLLTGALLVPVAASAQSHVDNPFVGASGYLNPDYSKEVNSSIVKVKDVQLKAKMQVVKSYPTSVWLDSINAIYGGSRNAGRLSLQGHLDAALAQKKANTPITVGFVIYDMPGRDCHALASNGELPLTQAGLQRYKTEYIDVIASTLANPKYKDLRIVNIIEPDSLPNLVTNQSTPACGQAASSGIYEAAIKYALDKLHATPNLYNYLDIGHSGWLGWDSNRSPAISLYTRVVQGTAAGLASADGFITNTANYTPLHEPNLPNPDLTIGGQPIRSSNFYQWNSFFDESTYAEALYNGFVGAGWSSKIGFLIDTGRNGWGGKARPASASGNDINTYVDSGRVDRRLHRGNWCNQSGAGIGMPPTAAPGGHIHALVWGKGPGESDGASKSIANNQGKGFDRYCDPTYTTPDGTLTGALPNAPIAGNWFHAQFLQLVANAYPAIGTSTKAALQSASTDAVPASRPTATKGLTANAADGEVRLSWSPVAGATGYTVQRVADATAAPITVASGLTSPSYVDQTLTNGTTYYYKVTANGASGADASSVTVSATPHR

SEQ ID NO: 50 - protein, 535 aa, Xanthomonas campestris
VRESDRSRSRDVIGAAMVFSVGTITPRDARTDQDARLTALGCRARFSREITMSIFRTASTLALATALALAAGPAFSYSINNSRQIVDDSGKVVQLKGVNVFGFETGNHVMHGLWARNWKDMIVQMQGLGFNAVRLPFCPATLRSDTMPASIDYSRNADLQGLTSLQILDKVIAEFNARGMYVLLDHHTPDCAGISELWYTGSYTEAQWLADLRFVANRYKNVPYVLGLDLKNEPHGAATWGTGNAATDWNKAAERGSAAVLAVAPKWLIAVEGITDNPVCSTNGGIFWGGNLQPLACTPLNIPANRLLLAPHVYGPDVFVQSYFNDSNFPNNMPAIWERHFGQFAGTHALLLGEFDGKYGEGDARDKTWQDALVKYLRSKGINQGFYWSWNPNSGDTGGILRDDWTSVRQDKMTLLRTLWGTAGNTTPTPTPTPTPTPTPTPTPTPTPTPGTSTFSTKVIVDNSWNGGYCNRVQVTNTGTASGTWSIAVPVTGTVNNAWNATWSQSGSTLRASGVDFNRTLAAGATAEFGFCAAS

SEQ ID NO: 51 - protein, 333 aa, Xanthomonas campestris
MLCIATMSIAQAQSPNRLKYAGVNISGAEIQSSKKPGVLNIDYRYPTASEYRYFAGKQMNIVRLPILWERMQPKAQGPLDQAQLALLKQAVANAKAANQYLIIDVHNYAKYYGQKIGSKRVPVRTFTDLWRRLAIAFKSDNAVIFGLMNEPYDISPESWAAAAQASIDSIRATGATNLILVPGALWSGAHSWYSTVAGQSNAVALANIRDPLNRYAIEVHQYLDTDSSGTSAGCVSRTIGAERLRSFTGWLRAQGKRGFLGEFGTANNATCTAALDGMLGYLETNHDVWIGWTFWAAGAWWNTSYPFNVQPDAQGRDKPQMKTLSARAHRVTR

SEQ ID NO: 52 - protein, 335 aa, Xanthomonas campestris
VFFLLLGVLPMGHAQTRALKYAGVNLAGAEFASSKKPGVLNKDYMYPASTDYSYFAGTGMNVIRLPILWERLQPAARGELDPAQLALVQQAVARAKASGMYLVLDIHNYSKYYGYKMGGPEVPLATFADLWRRLAVIFNSDNAVIFGLMNEPNNISASEWAGAAQAGIDAIRATGANNLILVPGALWTGAHSWHSLTSDGYSNATALASISDPLNRYAFEVHQYLDADSSGTSSVCVSETIGADRLRAFTEWLRTNNKRGFLGEFGTANNAVCNTALQGMLAYMENYADVWLGWTWWAAGAWWNTSYAYNVHPNKDGTDKPQMVILSPQAARATR

SEQ ID NO: 53 - protein, 343 aa, Xanthomonas campestris
ILPPYLLDHVAQAAPERARHCAQLTRHITAQLRQRRAQGLLARDSADDAPTATTDTAVQRHLYDAQQGTALPGVLVREEGAPPTDDVAVTEAYDYLGATHAFFQQVYARNSIDDAGMPLLGTVHYERNYDNAFWTGEQMVFGDGDGEIFTRFTIAIDVVAHELTHGVIERTANLIYQGQSGALNESVSDVFGVLVKQYALRQDAAQADWLVGAGMFLPGVQGVALRSMQAPGTAYDDPALGKDPQPAHMDAYVDTQEDDGGVHYNSGIPNRAFQRAAVAIGGYAWEKAGRIWYRALTGGALSASADFATFAALTVRVASTDYGAGSAEASAVEQAWRDVGVLA

SEQ ID NO: 54 - protein, 289 aa, Xanthomonas campestris
RVVLFLLTNFAVLILAGIVMSVLGVNPAQMSGLLVMAAIFGFGGSFISLLLSKFMAKRSTGAQVITEPRTQTERWLVDTVRRQAQAAGIGMPEVAIYDGPEINAFATGANRNNALVAVSTGLLQHMREDEAEAVLGHEIAHIANGDMVTMALLQGVLNTFVIVLARVVGGIIDSALSGNRDSGRGFAYYIIVFVLEMVFGLFATMIAMWFSRRREFRADAGGAQLAGRNKMIAALERLSLNHGQNTLPSQVQAFGISGGVGEGLRRLFLSHPPLTERIAALRASNGTAM

SEQ ID NO: 55 - protein, 580 aa, Xanthomonas campestris
MSTASLRKRTGSLTILGASALTSLLLAMPAFAGEVYLDGLATAQTHQKFIVTYKDGSTALASPSALTTSLRTAARAVPAKAGKALGLNSVRRLALGPELVRADRALDRAEAETLMRQLAADPNVQSVEVDQILHATLTPNDTRLSEQWAFGTTNAGLNIRPAWDKATGSGTVVAVIDTGITSHADLNANILAGYDFISDATTARDGNGRDSNAADEGDWYAANECGAGIPAASSSWHGTHVAGTVAAVTNNTTGVAGTAYGAKVVPVRVLGKCGGSLSDIADAIVWASGGTVSGIPANANPAEVINMSLGGGGSCSTTMQNAINGAVSRGTTVVVAAGNDASNVSGSLPANCANVIAVAATTSAGAKASYSNFGTGIDVSAPGSSILSTLNSGTTTPGSASYASYNGTSMASPHVAGVVALVQSVAPTALTPAAVETLLKNTARALPGACSGGCGAGIVNADAAVTAAINGGSGGGGGGGNTLTNGTPVTGLGAATGAELNYTITVPAGSGTLTVTTSGGSGDADLYVRAGSAPTDSAYTCRPYRSGNAETCTITAPSGTYYVRLKAYSTFSGVTLRASY

SEQ ID NO: 56 - protein, 483 aa, Xanthomonas campestris
MRRLSAGPMLLVADRRLDRVDSESLMRRLAADPTVKRVEVDVLMRPLLAPNDPGLPQQWAMGTTTASLNVRPAWDRTTGKGIVVAVIDTGITAHPDLAANVLPGYDFITDPTVAGDGNGRDNNAADQGDWSAANACGAGASASNSSWHGTHVAGIVAAVGNNAAGVVGTAFNAKLLPLRVLGKCGGYMSDIADAIVWASGGKVTGVPANPNPATVINLSLGGYGSCSTIIGNAITGAVTRGTAVVVAAGNSNMDVATSMPANCANVIAVAATTSAGAKASFSNFGKGVDIAAPGQAIISTLNSGTTVPANPAYAVYSGTSMAAPHVAGVVALMQSVALNPLTPATVEALLKSSARPLPVACAPGCGAGLVNADGAVAAVINATLLTSNAVRTGLSAAIGDSLYYQVKVPAGTRSLKVTLSGGSGNADLSLRANALPTDAAFGCRSMLVGNTEACTLTAPAAGTYYVRLKGTLAFSAVNLVATY

SEQ ID NO: 57 - protein, 549 aa, Xanthomonas campestris
SDPLFRYQWHLLNDGQEVIADTRPRAGTDLNVGPLHTLGLRGQGVTVAVVDDALELRHPDLVANVVPGGSKNFVDGSNDPTPRNGASHGTMVGGIMAAVGWNGLGGRGVAPDARLKGFNILSSEESPTDFDTNLRASWGDSVQSRDVDVFNNSFGSDLTYYPTISPAAERSLDRLMRRARNGKGGLYVQAAGNTFDSFTVLDDQGNWVERCPVLARTLGVTCSTPATDPLSNQPLIIATGAVNARGLRSSYSSAGAALWVTGFGGEFGLQRRYFGDRPRPALFDPAIVTTDLTGCAVGDNRDIAGQPPINALASSSSPIDASCNYSAAMNGTSAAAPTVAGVVALIVQANPSLTARDLKYILATSARQIDPAQQVIRYQGSVIEPGWITNAAGHAFSNWYGFGLVDAAEAVYRAYGFQPLPPQRDLGWKAARAGTSTIGGPNAAATLRLRLGDTLKIDTVQWSMQTTHKTPSNLRVVLISPSGTRSYVLTPFQALDTITQGAGFEIPLSTSNAFLDENVAGTWTLEVTDMTGSDVPAQLTGFKLRILGH

SEQ ID NO: 58 - protein, 348 aa, Xanthomonas campestris
SVLRRGVLGKVLVAVGIGDRLGRVADRQAACAGLTIHAVLGAGHHGEVAGLVHQRAGLGVGAGCRRAPERVADMDLVRLVRIDVSEGVRQIRFAAAVDRQLAIVLVQRVVDVDEVGADPAVARGVVGAEPGGVARPGAAAVEVGTRVLRVITRGSDATGGLVGAGGAAVEIVDAVDHGGRADCELALYTTSRAIQPNSERGAGGHCRQAVCGLHRAAIGQRGVLQRRAHLDGVVAAGGQVIGAGQIHLALDALARLQDGEVGCLGQAALDQAPGVVHRLAVAAHLQDAVVQRLQRQLIGRNAGNLATVVGDGDRDLALMSPGLVGHWRQFDGQRNRSAHGLCLRRTEH

SEQ ID NO: 59 - protein, 595 aa, Xanthomonas campestris
VVVAHSQGSMIAYDVLRQLQADGCEVALFLTLGSPLGLPQVRSMFKRWTGTRKLPFPDCVRRWVNVAETRDPIALDPDLTDDIANAKGRFENIAAARLNPDWEHNPHSGSGYLAIAQVRAAVRQAVGVGFDQPVSNAVLIKDLSEQLEAHGADYRHEVLIELDRRVLGADPAGVRAQLITHMRQIAGHSTGLDGDALDEAIELEDSLQRLISARLTRFEIETLQSRYRALGFRRVWRDAGKRALIHVSGNVLHVDAARTAYRARGQQIGWAVLDTGIAAAHPHFFVPGERDNVVAQWDCTRRGAPKRLTRADGKRFTALDGHGHGTHIAGIIAGCCRAAVPDASGKPGELLEFAGMAPETQLYGFKVLDDAGNGRDSWMIKAVQHVADLNERAGELVIHGVNLSLGGYFDPESYGCGFTPLCNELRRLWRQGVLVVVAAGNEGLAWLMQNDGGTYPANMDLSISDPGNLEDAIVVGSVHKSSPHNYGVSYFSSRGPTADGRSKPDVVAPGEKILSAYYGFDPRDPSSLMVEMSGTSMAAPHVSGVLAGFLSARREFIGFPDRVKQLLLDTCTDLQRDRYVQGRGVPNLMRMLGET

SEQ ID NO: 60 - protein, 627 aa, Xanthomonas campestris
VIDKNFRLNPLTGAILMMALGASGTLVAAPALQVKEPTQAAPAASDFSSRLIVRYKDGTAAASDRSSKLGVVQSAVGRAAPVTGARAGATAAKATYLRKLGIGSDLIKLSGTLTAAQVDKVVVELKNDPSVAEVQIDRMLRPVEIKKSVAATDVSPQLVPNDPLYAQYQWHLSNPNGGINAPAAWDLSQGAGVVVAVLDTGILPGHPDFAGNLLQGYDFITDAEVSRRPTDARVPGALDYGDWEEADNVCYDGSVAQESSWHGTHVSGTVAEATNNGLGMAGVAPKATILPVRVLGRCGGYTSDIADAIVWASGGTVDGVPANTNPAEVINMSLGGGEPCDPATQVAINGAVSRGTTVVVAAGNSGEDAANHSPASCNNTITVGATRITGGITYYSNYGSKVDLSGPGGGGSVDGNPGGYIWQAGYDGATTPTSGSYSYMGMGGTSMASPHVAGVVALVQSASIGLGDGPLTPAAMEALLKQASRRFPVTPPTSTPIGSGIVDAKAALEAVLVEPCDPDTETCAPAAIALTNKAPLTGLSGEYNSSTLYSFEAKAGAVLSFMTYGGTGNVSVYVSFEAEPTATDYDAKSTRPGNSETVRFTAPKAGTYYIKLVGAGDYAKLTLVARQ

SEQ ID NO: 61 - protein, 959 aa, Xanthomonas campestris
MLRPLSLLIAGVLGVAAGTVAPAAQAAPASLSKTANSSAIPDIAYTRFTLPNGLTVVVHEDHKAPVVAVSIWYHIGSGDEPAGKTGFAHLFEHLMFSGSENNKGSFFAPLEKVGTTDMNGTTWFDRTNYFETVPTTALDTALWLESDRMGHLLGAIGQEELDTQRGVVQNEKRQGENRPYGRVDQNILSNLFPANHPYQHDTIGSMEDLDAASLADVKQWFNDNYGAANTTLVLAGDITVAQARAKAEQYFGDIPSGKPVARQQPWVTPLAAQKRGVQHDHVSQPRIYRTWAAPQLGTDDMIQLDLATTVLGGGKTSRLYQRLVYQDQLVDDVSASIQPFALASQMQIQADVKDGVDPAKVEAIIDEELKKFLAEGPTADELQRAQVAYRAGFVRGLEKVGGFGGKAVILAEGQVYRGDPGAYKQDLQRGQAATIGSVKQAASTWFGKGDYLLTVLPAGKDFDPAAEDKAVVARGTEPGKPAPKLPAAAKYKVTASKLDRSKGVPDTASFPDLSFPQLQRGKLKNGVEVILAERHTIPVTQVELLFDAGYAADQGKKLGTASFSAALMNESTASLDSVEVAQRRQRLGAITEVGCDLDSCSASLNALNDQLQPSLQLFSDIVRNPAFKAADIERIRGQWLAGIAQEKTQPNSLGLRALPPLMYGDKHPYGIPLTGSGTEAAIKSLNAKDLQAFHNQWLRPDNLRILVAGDTTLAQIIPQLDAAFGDWTAPAAPLPKKNLVNVAAQPKPRVYLINRPDAPQSVILAGLLAPSTKAPDNLAITVANGAFGGTFTSRLNMNLRENKRWAYGARTQLVDAQGQRPYMFAAPVQTDKTAESANEIFKEATAIIGDKPLTTEEIEKIKNQRIRALPGSFETTGAVLGAIEGIVQFDRPDNYVQTLKPRLEAIDQPAAQAAIKDIIAPNAMTWVIVGDLKKIEAPVRALKLGEVQVLDTDGKPVKR

SEQ ID NO: 62 - protein, 428 aa, Xanthomonas campestris
MSEDRQGRSGDSNKILYCSFCGKSQHEVRKLIAGPSVFICDECVELCNDIIREELEEKAQSARSSLPKPREILEVLDQYVIGQLRAKRTLAVAVYNHYKRIESRSKNDEVELAKSNILLVGPTGSGKTLLAETLARLLNVPFTIADATTLTEAGYVGEDVENIIQKLLQKCDYDVEKAQQGIVYIDEIDKISRKSENPSITRDVSGEGVQQALLKLIEGTVASVPPQGGRKHPQQEFLQVDTKNILFICGGAFAGLDKVIQARSNDAGGIGFGAKVKSSERKQEVGKILAEVEPEDLIKFGLIPEFVGRLPVVATLEELDEPALIKILTEPKNAITKQFKKLFEMESVELEFRPDALSAIAKKALKRKTGARGLRTIVESVLLDTMYELPSQENVSKVVVDESVIEHKSEPYLIYQAQPAPAKAASGD

SEQ ID NO: 63 - protein, 205 aa, Xanthomonas campestris
VTKALNLVPMVVEQTSRGERAYDIYSRLLKERLIFLVGPIDDHMANVIVAQLLFLEADNPEKDISIYINSPGGVVTAGMAIYDTMQYIKPDVSTICVGQAASMGALLLASGAAGKRYALPNSRVMIHQPLGGFQGQATDIDIHAREILTLRSRLNEILAKHTGQSLETIARDTERDNFKSAVDAQAYGLVDQVLERRPEESIQPS

SEQ ID NO: 64 - protein, 191 aa, Xanthomonas campestris
VQAPLQIEEFLMTHSLSGKTVAVLATSGFEQSELQEPKRLLESWGATVEVIAPGDDAQIRGWNHTDWGDSVPVDTPLAQAKPDRYDALVLPGGVINPDNLRTNAQAIDFIRSVAASGKPVAAICHGPWLLVESGLVRDRKVTSWPSVKTDLSNAGGRWEDAEVVVDGQLITSRKPDDIPAFTDAVAKALAA

SEQ ID NO: 65 - protein, 658 aa, Xanthomonas campestris
MFSKDLEQTIGQCYKRAREARHEFMTVEHLLLSLLDNPSAQAVLKACGADQVRLHTDLEQAIEASVSRLAEDDGRDTQPTLGFQRVLQRAVYHVQSSGKKEVTGANVLVAIFGEKDSHAVYFLNQQDITRLDIVNYLSHGIAKLGEDGEQPSASDGEPKSDAGEGEGKGDALAEYATNLNDHARNGKIDPLVGRADEIERTIQVLCRRRKNNPLYVGEAGVGKTAIAEGLAKRIVDADVPEVLADAVIFSLDLGALVAGTKYRGDFEKRLKGVLTALKKVPNAVLFIDEIHTIIGAGSASGGTMDASNLIKPALASGELRCIGSTTFQEYRGIFEKDRALARRFQKIDIVEPTVGETFEILQGLKPKYEAHHGVTYADDALQAAVDLSVKHIGDRLLPDKAIDVIDEAGARQRLLPEGQRKELIDIEEIETIVAQDGAYSGQAGQRDRQGRAAASGTQSQDGDLRAEPGDRNAGWLDQAGALGPGQSGEADRQLPVRWPDRCGQDRGHQAARAAIGHRAGAVRHVRVHGSAFDQPSDRCASGLCRFRSGRSADRKDRQDAALRAAAGRGGEGASGHLQHPVAGHGPRHPHRYQRARSELQERDPGDDDQRRCHAGLASFDRFHQAGSFHRCDGIDPPRFHAGVPQPSGCDRAVPAAGL

SEQ ID NO: 66 - protein, 325 aa, Xanthomonas campestris
MARIPAKQVSATDKDVLQHLERNLKMVIFGQNPAIETLAGSIKLARSGLANPEKPIGNFLFAGPTGVGKTEVTKQLALQLGIELVRFDMSEYMEAHSISRLIGAPPGYVGFDQGGLLTEKIVKTPHCVLLLDEVEKAHPDIFNILLQVMDRGILTDTNGREANFKNVILVMTTNAGATQASRRSIGFTKQDHSTDAMESIRRGFTPEFRNRLDAIVQFQPLGFDHILRVVDKFIIELEMLLQEKHVSLSATPTARDWLAQHGFDPLMGARPMSRVIQEKIKRPLADELLFGKLVEGGRVNIDVKDGELVVEAHPEPERLLPATVD

SEQ ID NO: 67 - protein, 450 aa, Xanthomonas campestris
VPLDEIRRFVAVYNAVKQAYVDPVEDKKLMHAAVRGLLSDLDPHSTYFDKEDAEAFDEQATGAYDGIGVELLQQQDNTLKVIAPIDDTPAARAGVKAGDVIVAIDGKPIDASKAMEPLRGESGSKVTLTIVRDKVAKPFDITLQRETIRVASVRSKLLEPGYGYIRISTFQADTGADFQKNLKQLQAGGKLRGLVLDLRSNPGGLLTSAVQVADDLLDKGNIVSTRGRISISDAKFDATPGDLLGGAPVVVLVDAGSASASEVLAGALRDNQRARIIGSRTFGKGSVQTVLPLDNGDSVKLTTARYYTPSGKSIQASGIVPEVMLTPEPQPGDADVPASLTDFSEATLPGHLRGDAEGEEGYSAGDVLPGDGPINEALAELKQPGSVAKAQAARKAKAQAQKPKAIKPTPEPKPAAPRPPASEQTTPAEPTDKAKPAAPVPAAVPAEPVK

SEQ ID NO: 68 - protein, 727 aa, Xanthomonas campestris
MTYNVSASLKAGLLALVLTTPMALLARADTALPAAATPEQATATKLVYGLLSDSRYAYRPRTLDEAMSKDVFKRYLETLDGGKQFFTQADVDAFAPLQAGVGDALRGGNLEPAFQVFSVYKKRVDQRVKYARELLKQDFDFTGSEKFEYDRKDVPWAADDKQLDVLWRQSVMNDWLRLKLAGKKSDDIRKTLDKRYATLADSVNELKAEDAFQFFLNAYTNAVDPHTDYFTPRTAENFNQQMSLSLEGIGAQLQKQDDMVVIREVIPGGPAAVDGTLKPGDRIVGVGQTKSGAIEDVIGWRIDDVVAKIRGSKDTQVRLEYIPAELGIDGTHRTVTLTRQKVRLAEQAAKGETITLPASGDVPQRRIGIIKLPGFYQDFEGRRRNAADYASATRDVAKLLAGFKSDKVDGVVLDLRNNGGGSLDEAIELTGLFIEQGPVVQVRESGGRVTVNGDSNPKVAWDGPLGVLINRGSASASEIFAGAIQDYGRGLVIGETTFGKGTVQNIVDLDRWPAAEGQRYGQVKLTIAQFFRVSGSSTQHKGVVPDIAFPASVDATEFGESTYDNALPWTRIAAVPHTQYGNFAPLLPKLQTLHATRIASDKEFQWWEEDVKQFRDEKAKKYISLNEAERVAERQKQDQQRKDRQQVRKQLGLPLDPLADDSDDGLTGNERDIVKDTAREKAAEKRPDPLLHESAAILADALGLLSKDQPLSAQVLPQSTAPGRWAD

SEQ ID NO: 69 - protein, 803 aa, Xanthomonas campestris
VIVLLGLVLLAGGAWLATLGGSWYYAPAGLAMLIAGVLLFRGRNAGAWWFAAVVAATLLWTWWESGSDYWRWVPRLGLIVGLAFVLALLLPKLERPVSRAVSRSVAGGLAVVFVVAFALAFVPFGITEAEGALTQVAGIAAGVVNRPADGAQPAQPANAPVDDDWAAYGRSQAGQRYSPLTQINRDNVSQLQQAWVFHTGDVPTKRWGAETTPLKVGDSLYLCSARNQVIALDASTGKQRWRYDPKVKDEAIPYTAACRGVSYYEVPTAAPAANDAAASQAATAPALCRTRVIEGTLDGRLIALDARTGTPCADFGTHGQVDITVGMGETPPGYISINSPPAIVRGVVVIGHQVLDGQKRYEPSGVIQGFDAVTGQLRWAWDMTHPEWNGAPPPGQTWTRGTPNMWTTAAADEQLGYVYLPMGNSTADYWSSSRTPPENRYATSLVALDVTTGKPVWNFQTTHIDAWDYDLGSQPSLIDFPKDGVNVPAVLLPSKQGELYVLDRRTGEPLVGVEERAVPGGGVEPQMRYKTQPFSLYHTLRKPDLTERDMWGMTPIDQLVCRIQFRKASYKGIYTPPEADRHSIEYPGYNGGSDWGSVAVDPHRGVIVANYNDMPNYNILVPRAKADKLGWAPRDQVRGDAGGAEGAGDPQAGTPYAVNVNAGWRLPFTKLLCKQPPYGGIRAIDLKSGKTLWDRPFGSARGNGPFGIRSGLPIEIGTPNNGGSVVTAGGLIFIAAATDDLIRAIDLATGKELWHAKLPAGGQANPMVYAYGGREYLVIMAAGHHFMETPAGDALVAYALPQR